(19) United States
(12) Patent Application Publication
(10) Pub. No.: US 2025/0362304 A1
(43) Pub. Date: Nov. 27, 2025

(54) SYSTEMS AND METHODS FOR BIOMOLECULE ASSAYS

(71) Applicant: Seer, Inc., Redwood City, CA (US)

(72) Inventors: Xiaoyan ZHAO, Foster City, CA (US); Asim SIDDIQUI, San Francisco, CA (US); Chenyi ZHAO, Foster City, CA (US); Ryan William BENZ, Huntington Beach, CA (US); Mrittika BHATTACHARYA, San Jose, CA (US); Bryn Bo LEVITAN, San Mateo, CA (US); Biao LI, Foster City, CA (US)

(21) Appl. No.: 18/875,072

(22) PCT Filed: Jun. 14, 2023
(86) PCT No.: PCT/US2023/068457

§ 371 Date: Dec. 13, 2024

Related U.S. Application Data
(60) Provisional application No. 63/352,591, filed on Jun. 15, 2022.
(60) Provisional application No. 63/379,275, filed on Oct. 12, 2022.
(60) Provisional application No. 63/491,012, filed on Mar. 17, 2023.

(51) Int. Cl. G01N 33/68

(52) U.S. Cl. CPC G01N 33/6848; G01N 2570/00

(57) ABSTRACT

In some aspects, the present disclosure provides a method comprising: (a) selectively enriching a first plurality of biomolecule types in a first fluid composition; (b) selectively enriching a second plurality of biomolecule types in a second fluid composition; and (c) performing a downstream assay on biomolecule types of the first plurality of biomolecule types and the second plurality of biomolecule types, wherein the first fluid composition comprises a first pH, and the second fluid composition comprises a second pH, wherein the first pH and the second pH are different or the same. Also disclosed are systems, kits, compositions, and computer-readable media for performing biomolecule assays.

### CROSS-REFERENCE

This application claims the benefit of U.S. Provisional Application No. 63/352,591, filed Jun. 15, 2022, U.S. Provisional Application No. 63/379,275, filed Oct. 12, 2022, and U.S. Provisional Application No. 63/491,012, filed Mar. 17, 2023, each of which are incorporated herein by reference in their entirety.

### BACKGROUND

Biological samples such as biofluids contain a wide variety of proteins whose presence, processing, and relative abundances may be indicative of biological state. High abundance proteins and other proteins may overshadow the signal relative to other proteins in an assay. Sample preparation, such as dilution, can further overshadow the relative signals in an assay.

### SUMMARY

In some aspects, the present disclosure provides a method comprising: (a) selectively enriching a first plurality of biomolecule types in a first fluid composition; (b) selectively enriching a second plurality of biomolecule types in a second fluid composition; and (c) performing a downstream assay on biomolecule types of the first plurality of biomolecule types and the second plurality of biomolecule types, wherein the first fluid composition comprises a first pH, and the second fluid composition comprises a second pH, wherein the first pH and the second pH are different or the same.

In some embodiments, a second infinite-dilution limit enthalpy or free energy of solvation of a second biomolecule in the second subset of biomolecules is different when the second biomolecule is in the second fluid composition compared to when the second biomolecule is in the first fluid composition.

In some embodiments, the first fluid composition comprises a first set of intensive physical properties that mediates selective enrichment of the first plurality of biomolecule types, and the second fluid composition comprises a second set of intensive physical properties that mediates selective enrichment of the second plurality of biomolecule types, wherein the first set of intensive physical properties and the second set of intensive physical properties are different.

In some embodiments, the first fluid composition comprises a first ratio between a first sample volume and a first surface area of the first surface, and the second fluid composition comprises a second ratio between a second sample volume and a second surface area of the second surface, wherein the first ratio and the second ratio are different.

In some embodiments, the first fluid composition and the second fluid composition comprise different temperatures.

In some embodiments, the first fluid composition comprises a first ionic strength and the second fluid composition comprises a second ionic strength, wherein the first ionic strength and the second ionic strength are different.

In some embodiments, the first fluid composition and the second fluid composition comprise different salts.

In some embodiments, the first fluid composition and the second fluid composition comprises different solvents.

In some embodiments, the first fluid composition, the second fluid composition, or both comprise a buffer comprising tris(hydroxymethyl) aminomethane.

In some embodiments, the first fluid composition, the second fluid composition, or both comprise a buffer comprising citrate.

In some embodiments, the first fluid composition, the second fluid composition, or both comprise a pH between 2 and 4.

In some embodiments, the first fluid composition, the second fluid composition, or both comprise a pH between 5 and 7.

In some embodiments, the first fluid composition, the second fluid composition, or both comprise a pH between 9 and 10.

In some embodiments, the first fluid composition, the second fluid composition, or both comprise a pH of at least about 1, 2, 3, 4, 5, 6, 7, 8, 9, 10, 11, 12, 13, or 14.

In some embodiments, the first fluid composition, the second fluid composition, or both comprise a pH of at most about 1, 2, 3, 4, 5, 6, 7, 8, 9, 10, 11, 12, 13, or 14.

In some embodiments, the first fluid composition has a pH between 9 and 10, and the second fluid composition has a pH between 6 and 8.

In some embodiments, the first fluid composition and the second fluid composition have a pH between 9 and 10.

In some embodiments, the method further comprises diluting a sample to make the first composition, the second composition, or both.

In some embodiments, the diluting comprises adding a buffer.

In some embodiments, the buffer comprises a pH of at least about 1, 2, 3, 4, 5, 6, 7, 8, 9, 10, 11, 12, 13, or 14.

In some embodiments, the buffer comprises a pH of at most about 1, 2, 3, 4, 5, 6, 7, 8, 9, 10, 11, 12, 13, or 14.

In some embodiments, the sample and the buffer comprises different pH.

In some embodiments, the sample comprises at most about 1,000, 100, 10, 1, 0.1, 0.01, or 0.001 nanograms of biomolecules.

In some embodiments, the sample comprises at most about 1,000, 100, 10, 1, 0.1, 0.01, or 0.001 nanograms of biomolecules per mL of the sample.

In some embodiments, the sample comprises biomolecules from at most about 1,000, 100, 10, or 1 cell.

In some embodiments, the sample comprises at most about 1,000, 100, 10, 1, 0.1, 0.01, or 0.001 microliters.

In some embodiments, the sample comprises a complex biological sample.

In some embodiments, the complex biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises plasma.

In some embodiments, the selectively enriching the first plurality of biomolecule types in the comprises contacting the first fluid composition with a first surface to adsorb the first plurality of biomolecule types on the first surface.

In some embodiments, the selectively enriching the second plurality of biomolecule types in the comprises contacting the second fluid composition with a second surface to adsorb the second plurality of biomolecule types on the second surface.

In some embodiments, the first surface, the second surface, or both comprise a particle.

In some embodiments, the particle is a porous particle.

In some embodiments, the particle is a microparticle.

In some embodiments, the particle is a nanoparticle.

In some embodiments, the particle comprises a magnetic material.

In some embodiments, the particle comprises a paramagnetic material.

In some embodiments, the paramagnetic material is a superparamagnetic material.

In some embodiments, the paramagnetic material comprises iron oxide.

In some embodiments, the selectively enriching the first plurality of biomolecule types in the comprises contacting the first fluid composition with a first plurality of surface types to adsorb the first plurality of biomolecule types on the first plurality of surface types.

In some embodiments, the selectively enriching the second plurality of biomolecule types in the comprises contacting the second fluid composition with a second plurality of surface types to adsorb the second plurality of biomolecule types on the second plurality of surface types.

In some embodiments, the first plurality of surface types have the same charge.

In some embodiments, the second plurality of surface types have the same charge.

In some embodiments, the first plurality of surface types have the same charge, and the second plurality of surface types have the same charge.

In some embodiments, the charge of the first plurality of surface type is opposite the charge of the second plurality of surface types.

In some embodiments, the first plurality of surface types have the same sign of zeta potential.

In some embodiments, the second plurality of surface types have the same sign of zeta potential.

In some embodiments, the first plurality of surface types, and the second plurality of surface types have the same sign of zeta potential.

In some embodiments, the first plurality of surface types each have a zeta potential of less than −5 mV, less than −10 mV, less than −15 mV, or less than −20 mV.

In some embodiments, the second plurality of surface types each have a zeta potential of less than −5 mV, less than −10 mV, less than −15 mV, or less than −20 mV.

In some embodiments, the first plurality of surface types each have a zeta potential of more than 5 mV, more than 10 mV, more than 15 mV, or more than 20 mV.

In some embodiments, the second plurality of surface types each have a zeta potential of more than 5 mV, more than 10 mV, more than 15 mV, or more than 20 mV.

In some embodiments, the first plurality of surface types, the second plurality of surface types, or both comprise an acidic functional group.

In some embodiments, the acidic functional group comprises a Bronsted-Lowry acid or a Lewis acid functional group.

In some embodiments, the first plurality of surface types, the second plurality of surface types, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a thiol group, a polymer, or any combination thereof.

In some embodiments, the first plurality of surface types comprises a first surface comprising a carboxylate-functionalized surface and second surface comprising a silanol-functionalized surface.

In some embodiments, the first plurality of surface types, the second plurality of surface types, or both comprise a primary amine group, a secondary amine group, a tertiary amine group, a quaternary amine group, a cyclic secondary amine group, a primary amide group, a secondary amide group, a tertiary amide group, an imine group, a pyridyl group, a pyrimidine group, a pyrrolidinium group, an imidazole group, a guanidine group, a guanidinium group, a carbamoyl group, an ammonium group, a pyridinium group, or any combination thereof.

In some embodiments, the first plurality of surface types each comprise an amine group.

In some embodiments, the method further comprises washing the first plurality of biomolecule types with a first wash composition and washing the second plurality of biomolecule types with a second wash composition.

In some embodiments, the first fluid composition and the first wash composition comprise at least one common intensive physical property.

In some embodiments, the first fluid composition and the first wash composition comprise at least one common solvent.

In some embodiments, the first fluid composition and the first wash composition comprise at least one different intensive physical property.

In some embodiments, the second fluid composition and the second wash composition comprise at least one common intensive physical property.

In some embodiments, the second fluid composition and the second wash composition comprise at least one common solvent.

In some embodiments, the second fluid composition and the second wash composition comprise at least one different intensive physical property.

In some embodiments, the first wash composition and the second wash composition comprise at least one common intensive physical property.

In some embodiments, the first wash composition and the second wash composition are the same.

In some embodiments, the first wash composition and the second wash composition comprises at least one different intensive physical property.

In some embodiments, the first wash composition releases the first plurality of biomolecule types adsorbed on the first surface from the first surface.

In some embodiments, the method further comprises purifying the first plurality of biomolecule types to produce a first purified composition.

In some embodiments, the purifying comprises drying the plurality of biomolecule types to remove the first wash composition.

In some embodiments, the method further comprises reconstituting the first purified composition with a first reconstitution composition to produce a first reconstituted composition.

In some embodiments, the second wash composition releases the second plurality of biomolecule types deposited on the second surface from the second surface.

In some embodiments, the method further comprises purifying the second plurality of biomolecule types to produce a second purified composition.

In some embodiments, the purifying comprises drying the second plurality of biomolecule types to remove the second wash composition.

In some embodiments, the method further comprises reconstituting the second purified composition with a second reconstitution composition to produce a second reconstituted composition.

In some embodiments, the downstream assay comprises mass spectrometry.

In some embodiments, the mass spectrometry comprises LC-MS/MS.

In some embodiments, the downstream assay comprises protein sequencing.

In some embodiments, the downstream assay comprises contacting the first plurality of biomolecule types, the second plurality of biomolecule types, or both with a pair of antibodies capable of binding to at least one biomolecule type in the first plurality of biomolecule types, the second plurality of biomolecule types, or both, wherein the pair of antibodies comprises complementary single-stranded nucleic acid sequences attached thereto, such that when the pair of antibodies bind to the at least one biomolecule type, the complementary nucleic acids hybridize to form a double stranded nucleic acid, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

In some embodiments, the downstream assay comprises contacting the first plurality of biomolecule types, the second plurality of biomolecule types, or both with one or more aptamers capable of binding to at least one biomolecule type in the first plurality of biomolecule types, the second plurality of biomolecule types, or both, wherein the one or more aptamers are coupled to a surface via a cleavable linker.

In some embodiments, the surface is a particle surface.

In some embodiments, the cleavable linker is photocleavable.

In some embodiments, the method further comprises contacting the first plurality of biomolecule types, the second plurality of biomolecule types, or both with a macromolecular competitor configured to, in a fluid composition, reduce dissociation of a complex comprising the one or more aptamers and the first plurality of biomolecule types, the second plurality of biomolecule types, or both.

In some embodiments, the macromolecular competitor is further configured to bind to a biomolecule type that is different from the first plurality of biomolecule types, the second plurality of biomolecule types, or both.

In some embodiments, the macromolecular competitor is a polyanionic macromolecule.

In some embodiments, the downstream assay comprises nucleic acid sequencing.

In some embodiments, (a) and (b) are performed on one machine.

In some embodiments, (a) and (b) are performed on different machines.

In some embodiments, (a) and (b) are performed in parallel.

In some embodiments, (a) and (b) are performed in series.

In some embodiments, the first fluid composition and the second fluid composition are a portion of the same sample.

In some embodiments, the first fluid composition and the second fluid composition are from different samples.

In some embodiments, the selectively enriching is performed for the first fluid composition and the second fluid composition in parallel.

In some embodiments, the selectively enriching is performed for the first fluid composition and the second fluid composition in serial.

In some embodiments, the selectively enriching is performed at a rate of at least 100, 1,000, 10,000, 100,000, or 1,000,000 biomolecules enriched per hour.

In some embodiments, the selectively enriching is performed at a rate of at least 1, 10, 100, 1,000, 10,000, 100,000, 1,000,000, or 10,000,000 μg of biomolecules enriched per hour.

In some embodiments, the contacting is performed for the first fluid composition and the second fluid composition in parallel.

In some embodiments, the contacting is performed for the first fluid composition and the second fluid composition in serial.

In some embodiments, the contacting is performed at a rate of at least 100, 1,000, 10,000, 100,000, or 1,000,000 biomolecules deposited per hour.

In some embodiments, the contacting is performed at a rate of at least 1, 10, 100, 1,000, 10,000, 100,000, 1,000,000, or 10,000,000 μg of biomolecules deposited per hour.

In some embodiments, the washing is performed for the first fluid composition and the second fluid composition in parallel.

In some embodiments, the washing is performed for the first fluid composition and the second fluid composition in serial.

In some embodiments, the washing is performed at a rate of at least 100, 1,000, 10,000, 100,000, or 1,000,000 biomolecules washed per hour.

In some embodiments, the washing is performed at a rate of at least 1, 10, 100, 1,000, 10,000, 100,000, 1,000,000, or 10,000,000 μg of biomolecules washed per hour.

In some embodiments, the purifying is performed for the first fluid composition and the second fluid composition in parallel.

In some embodiments, the purifying is performed for the first fluid composition and the second fluid composition in serial.

In some embodiments, the purifying is performed at a rate of at least 100, 1,000, 10,000, 100,000, or 1,000,000 purified per hour.

In some embodiments, the purifying is performed at a rate of at least 1, 10, 100, 1,000, 10,000, 100,000, 1,000,000, or 10,000,000 μg of biomolecules purified per hour.

In some embodiments, the reconstituting is performed for the first fluid composition and the second fluid composition in parallel.

In some embodiments, the reconstituting is performed for the first fluid composition and the second fluid composition in serial.

In some embodiments, the reconstituting is performed at a rate of at least 100, 1,000, 10,000, 100,000, or 1,000,000 reconstituted per hour.

In some embodiments, the reconstituting is performed at a rate of at least 1, 10, 100, 1,000, 10,000, 100,000, 1,000,000, or 10,000,000 μg of biomolecules reconstituted per hour.

In some embodiments, the downstream assay is performed for the first fluid composition and the second fluid composition in parallel.

In some embodiments, the downstream assay is performed for the first fluid composition and the second fluid composition in serial.

In some embodiments, the downstream assay is performed at a rate of at least 1, 2, 3, 4, 5, 6, 7, 8, 9, 10, 100 samples assayed per hour.

In some embodiments, the downstream assay is performed at a rate of at least 1, 10, 100, 1,000, 10,000, 100,000, 1,000,000, or 10,000,000 μg of biomolecules assayed per hour.

In some embodiments, the downstream assay is performed at a rate of at least 100, 1,000, 10,000, 100,000, or 1,000,000 biomolecules identified per hour.

In some embodiments, the downstream assay is performed at a rate of at least 100, 1,000, 10,000, 100,000, or 1,000,000 protein groups identified per hour.

In some embodiments, the method is performed for the first fluid composition and the second fluid composition in parallel.

In some embodiments, the method is performed for the first fluid composition and the second fluid composition in serial.

In some embodiments, the method is performed at a rate of at least 1, 2, 3, 4, 5, 6, 7, 8, 9, 10, 100 samples per hour.

In some embodiments, the method is performed at a rate of at least 1, 10, 100, 1,000, 10,000, 100,000, 1,000,000, or 10,000,000 μg of biomolecules assayed per hour.

In some embodiments, the method is performed at a rate of at least 100, 1,000, 10,000, 100,000, or 1,000,000 biomolecules identified per hour.

In some embodiments, the method is performed at a rate of at least 100, 1,000, 10,000, 100,000, or 1,000,000 protein groups identified per hour.

In some embodiments, the downstream assay identifies at least about 100, 1,000, 10,000, 100,000, or 1,000,000 biomolecules when the first fluid composition and the second fluid composition is a HeLa cell extract

In some embodiments, the first plurality of biomolecule types and the second plurality of biomolecule types together comprise at least about 6, 7, 8, 9, 10, 11, 12, 13, 14, 15, or 16 dynamic range when the downstream assay is performed on the first fluid composition and the second fluid composition before the selectively enriching in (a) and (b).

In some embodiments, the first plurality of biomolecule types is depleted in at least a first biomolecule and enriched in a second biomolecule, wherein the first biomolecule is more abundant than the second biomolecule in the first fluid composition.

In some embodiments, the second plurality of biomolecule types is depleted in at least a first biomolecule and enriched in a second biomolecule, wherein the first biomolecule is more abundant than the second biomolecule in the second fluid composition.

In some embodiments, a first likelihood that the downstream assay identifies a low abundance biomolecule in the first plurality of biomolecule types or the second plurality of biomolecule types is larger than a second likelihood that the downstream assay identifies the low abundance biomolecule in the first fluid composition or the second fluid composition, wherein the low abundance biomolecule comprises less than about 1, 10⁻¹, 10⁻², 10⁻³, 10⁻⁴, 10⁻⁵, 10⁻⁶, 10⁻⁷, 10⁻⁸, 10⁻⁹, or 10⁻¹⁰ percent by mass of the first set of biomolecules or the second set of biomolecules.

In some embodiments, the first likelihood is larger than the second likelihood by a factor of at least 2, 5, 10, 102, 102, 103, 104, 105, 106, 107, 108, 109, or 1010.

In some embodiments, the downstream identifies one or more biomolecules in the first plurality of biomolecule types, the second plurality of biomolecule types, or both, with a coefficient of variance of at most about 30, 25, 20, 15, 10, 5, 4, 3, 2, or 1 percent.

In some embodiments, the downstream identifies one or more biomolecules in the first plurality of biomolecule types, the second plurality of biomolecule types, or both, with a coefficient of variance of at least about 30, 25, 20, 15, 10, 5, 4, 3, 2, or 1 percent.

In some embodiments, the one or more biomolecules comprise at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 peptides.

In some embodiments, the one or more biomolecules comprise at least about 100, 200, 300, 400, 500, 600, 700, 800, 900, 1,000, 2,000, 3,000, 4,000, 5,000, 6,000, 7,000, 8,000, 9,000, or 10,000 proteins.

In some embodiments, the first plurality of biomolecule types, the second plurality of biomolecule types, or both comprise one or more polyamino acids.

In some embodiments, the method further comprises identifying a biological state associated with the first fluid composition based at least partially on one or more physical properties of the first fluid composition.

In some embodiments, the method further comprises identifying a biological state associated with the second fluid composition based at least partially on one or more physical properties of the second fluid composition.

In some embodiments, the method further comprises identifying a biological state associated with the first fluid composition based at least partially on one or more physical properties of the first wash composition.

In some embodiments, the method further comprises identifying a biological state associated with the second fluid composition based at least partially on one or more physical properties of the second wash composition.

In some embodiments, the method further comprises identifying a biological state associated with the first fluid composition based at least partially on one or more physical properties of the first reconstitution composition.

In some embodiments, the method further comprises identifying a biological state associated with the second composition based at least partially on one or more physical properties of the second reconstitution composition.

In some embodiments, the first surface is disposed in a first lyophilized composition comprising (i) at least one nanoparticle, and (ii) one or more modifiers.

In some embodiments, the second surface is disposed in a second lyophilized composition comprising (i) at least one nanoparticle, and (ii) one or more modifiers.

In some embodiments, the one or more modifiers comprise pH modifiers, ionic strength modifiers, viscosity modifiers, or any combination thereof.

In some embodiments, the first pH and the second pH are different. In some embodiments, the first pH and the second pH are the same.

In some aspects, the present disclosure provides a method comprising: (a) selectively enriching a first plurality of biomolecule types in a first fluid composition; (b) selectively enriching a second plurality of biomolecule types in a second fluid composition; and (c) performing a downstream assay on biomolecule types of the first plurality of biomolecule types and the second plurality of biomolecule types, wherein the first fluid composition and the second fluid composition are different such that a first infinite-dilution limit enthalpy or free energy of solvation of a first biomolecule in the first plurality of biomolecule types is different when the first biomolecule is in the first fluid composition compared to when the biomolecule is in the second fluid composition.

In some aspects, the present disclosure provides a kit comprising: a first reagent comprising a first pH; a second reagent comprising a second pH; a first surface configured to selectively enrich a first plurality of biomolecule types in a first fluid composition comprising the first surface and the first reagent; and a second surface configured to selectively enrich a second plurality of biomolecule types in a second fluid composition comprising the second surface and the second reagent, wherein the first fluid composition comprises the first reagent, and the second fluid composition comprises the second reagent, wherein the first pH and the second pH are different or the same.

In some aspects, the present disclosure provides a composition comprising: a suspension comprising: a first particle comprising (i) a first paramagnetic portion and (ii) a first surface chemistry; a second particle comprising (i) a second paramagnetic portion and (ii) a second surface chemistry, wherein the second surface chemistry and the first surface chemistry are different; and a plurality of biomolecules adsorbed on the first particle and the second particle.

In some embodiments, the first particle and the second particle have the same charge.

In some embodiments, the first particle and the second particle have the same sign of zeta potential.

In some embodiments, the same sign is a negative sign.

In some embodiments, the same sign is a positive sign.

In some embodiments, the first particle and the second particle each have a zeta potential of less than −5 mV, less than −10 mV, less than −15 mV, or less than −20 mV.

In some embodiments, the first particle and the second particle each have a zeta potential of more than 5 mV, more than 10 mV, more than 15 mV, or more than 20 mV.

In some embodiments, the first particle, the second particle, or both comprise an acidic functional group.

In some embodiments, the acidic functional group comprises a Bronsted-Lowry acid or a Lewis acid functional group.

In some embodiments, the first particle, the second particle, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a thiol group, a polymer, or any combination thereof.

In some embodiments, the first particle, the second particle, or both comprise a carboxylate-functionalization, a silanol-functionalization, or both.

In some embodiments, the first particle, the second particle, or both comprise a primary amine group, a secondary amine group, a tertiary amine group, a quaternary amine group, a cyclic secondary amine group, a primary amide group, a secondary amide group, a tertiary amide group, an imine group, a pyridyl group, a pyrimidine group, a pyrrolidinium group, an imidazole group, a guanidine group, a guanidinium group, a carbamoyl group, an ammonium group, a pyridinium group, or any combination thereof.

In some embodiments, the first particle, the second particle, or both comprise an amine group.

In some embodiments, the first surface chemistry, the second surface chemistry, or both comprise an acidic functional group.

In some embodiments, the acidic functional group comprises a Bronsted-Lowry acid or a Lewis acid functional group.

In some embodiments, the first surface chemistry, the second first surface chemistry, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

In some embodiments, the suspension is stable for at least about 1, 5, 10, 15, 30, or 60 minutes.

In some embodiments, a time constant for destabilization of the suspension is at least about 1, 5, 10, 15, 30, or 60 minutes.

In some embodiments, a mean aggregation number of the first particle and the second particle in the suspension is at most about 1,000, 100, or 10.

In some embodiments, the suspension comprises at least about 1, 2, 3, 4, 5, 6, 7, 8, 9, or 10 parts of the first particle to about 1 part of the second particle.

In some embodiments, the suspension comprises at most about 1, 2, 3, 4, 5, 6, 7, 8, 9, or 10 parts of the first particle to about 1 part of the second particle.

In some embodiments, the suspension comprises about 15 parts of the first particle to about 6 parts of the second particle.

In some embodiments, the parts are parts by weight, parts by volume, or parts by surface area.

In some embodiments, the parts are parts by weight.

In some embodiments, the suspension comprises at least about 2, 3, 4, 5, 6, 7, 8, 9, or 10 distinct particles.

In some embodiments, suspension comprises 2, 3, 4, 5, 6, 7, 8, 9, or 10 distinct particles.

In some embodiments, the first particle, the second particle, or both are a nanoparticle.

In some embodiments, the first particle, the second particle, or both are a microparticle.

In some embodiments, the first particle, the second particle, or both are porous.

In some embodiments, a first size of the first particle is at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of the second particle.

In some embodiments, a first size of the first particle is at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of the second particle.

In some embodiments, a size of the first particle is within ±40% of a size of the second particle, a size of the first particle is within ±30% of a size of the second particle, a size of the first particle is within ±25% of a size of the second particle, a size of the first particle is within ±20% of a size of the second particle, a size of the first particle is within ±15% of a size of the second particle, or a size of the first particle is within ±10% of a size of the second particle,

In some embodiments, the first size is a first diameter, and the second size is a second diameter.

In some embodiments, the first size is a first average size, and the second size is a second average size.

In some embodiments, the first average size and the second average size are mean sizes or median sizes.

In some embodiments, a ratio of a mean diameter for the first particle to a mean diameter of the second particle is about 3:2 to about 2:3.

In some embodiments, the plurality of biomolecules comprises at most about 1,000, 100, 10, 1, 0.1, 0.01, or 0.001 nanograms of biomolecules.

In some embodiments, the plurality of biomolecules comprises at most about 1,000, 100, 10, 1, 0.1, 0.01, or 0.001 nanograms of biomolecules per mL.

In some embodiments, the plurality of biomolecules comprises biomolecules from at most about 1,000, 100, 10, or 1 cell.

In some aspects, the present disclosure provides a method comprising: assaying the plurality of biomolecules in a composition disclosed herein to identify one or more biomolecules in the plurality of biomolecules.

In some embodiments, the assaying comprises performing mass spectrometry.

In some embodiments, the mass spectrometry comprises LC-MS/MS.

In some embodiments, the assaying comprises performing protein sequencing.

In some embodiments, the assaying comprises contacting the plurality of biomolecules with a pair of antibodies capable of binding to at least one biomolecule in the plurality of biomolecules, wherein the pair of antibodies comprises complementary single-stranded nucleic acid sequences attached thereto, such that when the pair of antibodies bind to the at least one biomolecule, the complementary nucleic acids hybridize to form a double stranded nucleic acid, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

In some embodiments, the assaying comprises contacting the plurality of biomolecules with one or more aptamers capable of binding to at least one biomolecule in the plurality of biomolecules, wherein the one or more aptamers are coupled to a surface via a cleavable linker.

In some embodiments, the surface is a particle surface.

In some embodiments, the cleavable linker is photocleavable.

In some embodiments, the method further comprises contacting the plurality of biomolecules with a macromolecular competitor configured to, in a fluid composition, reduce dissociation of a complex comprising the one or more aptamers and the biomarker.

In some embodiments, the macromolecular competitor is further configured to bind to a biomolecule that is different from the biomarker.

In some embodiments, the macromolecular competitor is a polyanionic macromolecule.

In some embodiments, the assaying comprises performing nucleic acid sequencing.

In some embodiments, a sample comprises the plurality of biomolecules.

In some embodiments, the sample is a complex biological sample.

In some embodiments, the complex biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises plasma.

In some embodiments, the plurality of biomolecules comprises one or more polyamino acids.

In some embodiments, the assaying is performed at a rate of 1, 2, 3, 4, 5, 6, 7, 8, 9, 10, or 100 samples assayed per hour.

In some embodiments, the assaying is performed at a rate of at least 1, 10, 100, 1,000, 10,000, 100,000, 1,000,000, or 10,000,000 μg of biomolecules assayed per hour.

In some embodiments, the assaying is performed at a rate of at least 1, 10, 100, 1,000, 10,000, 100,000, or 1,000,000 biomolecules identified per hour.

In some embodiments, the assaying is performed at a rate of at least 1, 10, 100, 1,000, 10,000, 100,000, or 1,000,000 protein groups identified per hour.

In some embodiments, the assaying identifies at least about 100, 1,000, 10,000, or 100,000 biomolecules when the suspension comprises a HeLa cell extract

In some embodiments, the plurality of biomolecules comprises at least about 6, 7, 8, 9, 10, 11, 12, 13, 14, 15, or 16 dynamic range when the assaying is performed on the plurality of biomolecules in a biological sample in the absence of the first particle and the second particle.

In some embodiments, a first likelihood that the assaying identifies a low abundance biomolecule in the plurality of biomolecules is larger than a second likelihood that the assaying identifies the low abundance biomolecule in a biological sample in the absence of the first particle and the second particle, wherein the low abundance biomolecule comprises less than about 1, 10⁻¹, 10⁻², 10⁻³, 10⁻⁴, 10⁻⁵, 10⁻⁶, 10⁻⁷, 10⁻⁸, 10⁻⁹, or 10⁻¹⁰ percent by mass of the first set of biomolecules or the second set of biomolecules.

In some embodiments, the first likelihood is larger than the second likelihood by a factor of at least 2, 5, 10, 102, 102, 103, 104, 105, 106, 107, 108, 109, or 1010.

In some embodiments, the assaying identifies one or more biomolecules in the plurality of biomolecules with a coefficient of variance of at most about 30, 25, 20, 15, 10, 5, 4, 3, 2, or 1 percent.

In some embodiments, the assaying identifies one or more biomolecules in the plurality of biomolecules with a coefficient of variance of at least about 30, 25, 20, 15, 10, 5, 4, 3, 2, or 1 percent.

In some aspects, the present disclosure provides a system for analyzing a plurality of biological samples, comprising: (a) a plurality of partitions comprising a first partition and a second partition; (b) a plurality of reagent storages comprising a first reagent comprising a first pH and a second reagent comprising a second pH, wherein the first pH and the second pH are different or the same; (c) a plurality of substrates comprising a first substrate comprising a first surface chemistry and a second substrate comprising a second surface chemistry; (d) one or more transfer devices operably connected to the plurality of partitions, the plurality of reagent storages, and the plurality of substrates; and (e) a computer comprising at least one processor and instructions executable by the at least one processor to perform steps comprising: (i) generating, using the one or more transfer devices, a first fluid composition in the first partition comprising the first substrate, the first reagent, and a first plurality of biomolecules, wherein the first plurality of biomolecules is adsorbed on the first substrate; (ii) generating, using the one or more transfer devices, a second fluid composition in the second partition comprising the second substrate, the second reagent, and a second plurality of biomolecules, wherein the second plurality of biomolecules is adsorbed on the second substrate; and (iii) preparing, using the one or more transfer devices, the first plurality of biomolecules and the second plurality of biomolecules for mass spectrometry.

In some aspects, the present disclosure provides a system for analyzing a plurality of biological samples, comprising: (a) a partition; (b) a reagent storage comprising a reagent; (c) a plurality of substrates comprising a first substrate comprising a first surface chemistry and a second substrate comprising a second surface chemistry, wherein the first surface chemistry and the second surface chemistry are different; (d) one or more transfer devices operably connected to the partition, the reagent storage, and the plurality of substrates; and (e) a computer comprising at least one processor and instructions executable by the at least one processor to perform steps comprising: (i) generating, using the one or more transfer devices, a fluid composition in the partition comprising the plurality of substrates, the reagent, and a plurality of biomolecules, wherein the plurality of biomolecules is adsorbed on the substrate; and (ii) preparing, using the one or more transfer devices, the plurality of biomolecules for mass spectrometry.

In some aspects, the present disclosure provides a method comprising: (a) forming a first suspension comprising a first plurality of particles with a first portion of a biological sample, wherein the first plurality of particles comprise a first particle and second particle, wherein the first particle has a first functionalized surface chemistry that is different from a second functionalized surface chemistry of the second particle, and wherein both the first particle and the second particle have a negative charge; (b) forming a second suspension comprising a second plurality of particles with a second portion of the biological sample, wherein the second plurality of particles comprises a third particle and fourth particle, wherein third particle has a third functionalized surface chemistry that is different from a fourth functionalized surface chemistry of the fourth particle, and wherein both the third particle and the fourth particle have a positive charge; (c) enriching for biomolecules that have adsorbed to the first plurality of particles and the second plurality of parties; and (d) performing a downstream assay on the enriched biomolecules.

In some embodiments, the first suspension and the second suspension each have a pH between 9 and 10.

In some embodiments, the first suspension and the second suspension each comprise Tris buffer.

In some embodiments, the first suspension has a pH between 9 and 10.

In some embodiments, the first suspension comprises Tris buffer.

In some embodiments, the second suspension has a pH between 6 and 8.

In some embodiments, the first particle is a magnetic particle and comprises an outer layer with a silanol surface functionalization, and the second particle is a magnetic particle and comprises an outer layer with a carboxylate surface functionalization.

In some embodiments, the third particle is a magnetic particle and comprises an outer layer with an amine surface functionalization, and the fourth particle is a magnetic particle and comprises an outer layer with an amine surface functionalization.

In some embodiments, the outer layer of the third particle comprises a polymer.

In some embodiments, the first plurality of particles has a ratio of about 10 to about 15 parts by weight of the first particle to about 1 part by weight of the second particle.

In some embodiments, the second plurality of particles has a ratio about 10 to about 15 parts by weight of the third particle to about 1 part by weight of the fourth particle.

In some embodiments, a ratio of a mean diameter for the first particle to a mean diameter of the second particle is about 3:2 to about 2:3

In some embodiments, a ratio of a mean diameter for the third particle to a mean diameter of the fourth particle is about 3:2 to about 2:3.

In some embodiments, each of the first particle, the second particle, the third particle, and the fourth particle are superparamagnetic iron oxide nanoparticles.

In some aspects, the present disclosure provides a kit comprising: (a) a first composition comprising: a first particle and second particle, wherein first particle has a first functionalized surface chemistry that is different from a second functionalized surface chemistry of the second particle, and wherein both the first particle and the second particle have a negative charge, wherein the first composition is a solid; and (b) a second composition comprising: a third particle and fourth particle, wherein third particle has a third functionalized surface chemistry that is different from a fourth functionalized surface chemistry of the fourth particle, and wherein both the third particle and the fourth particle have a positive charge, wherein the second composition is a solid.

In some embodiments, the kit further comprises a buffer having a pH between 9 and 10.

In some embodiments, the kit further comprises a Tris buffer having a pH between 9 and 10.

In some embodiments, both the first composition and the second composition in the kit are lyophilized.

In some embodiments, the first particle in the kit is a magnetic particle and comprises an outer layer with a silanol surface functionalization, and the second particle in the kit is a magnetic particle and comprises an outer layer with a carboxylate surface functionalization.

In some embodiments, the third particle in the kit is a magnetic particle and comprises an outer layer with an amine surface functionalization, and the fourth particle in the kit is a magnetic particle and comprises an outer layer with an amine surface functionalization.

In some embodiments, the outer layer of the third particle in the kit comprises a polymer.

In some embodiments, the first composition in the kit has a ratio of about 10 to about 15 parts by weight of the first particle to about 1 part by weight of the second particle.

In some embodiments, the second composition in the kit has a ratio of about 10 to about 15 parts by weight of the third particle to about 1 part by weight of the fourth particle.

In some embodiments, a ratio of a mean diameter for the first particle in the kit to a mean diameter of the second particle in the kit is about 3:2 to about 2:3.

In some embodiments, a ratio of a mean diameter for the third particle in the kit to a mean diameter of the fourth particle in the kit is about 3:2 to about 2:3.

In some embodiments, each of the first particle, the second particle, the third particle, and the fourth particle in the kit are superparamagnetic iron oxide nanoparticles.

In some aspects, the present disclosure provides a suspension comprising: (a) first particles comprising outer layer with a negatively charged surface functionalization; (b) a biological sample; and (c) a buffer configured to maintain a pH of the suspension between 9 and 10, wherein the biomolecules are adsorbed to the outer layer of the first particles.

In some aspects, the present disclosure provides a suspension comprising: (a) first particles comprising a first outer layer with a negatively charged surface functionalization; (b) second particles comprising a second outer layer with a negatively charged surface functionalization, wherein the second outer layer is different from the first outer layer; (c) a biological sample; and (d) a buffer configured to maintain a pH of the suspension between 9 and 10, wherein the biomolecules are adsorbed to the first outer layer of the first particles and the second outer layer of the particles.

In some embodiments, the first outer layer comprises silanol, and the second outer layer comprises a carboxylate.

In some aspects, the present disclosure provides a suspension comprising: (a) first particles comprising a first outer layer with a positively charged surface functionalization; (b) second particles comprising a second outer layer with a positively charged surface functionalization, wherein the second outer layer is different from the first outer layer; (c) a biological sample; and (d) a buffer configured to maintain a pH of the suspension between 9 and 10, wherein the biomolecules are adsorbed to the first outer layer of the first particles and the second outer layer of the particles.

In some embodiments, the first and second outer layer comprise an amine group.

In some embodiments, the biomolecules comprise at least 100 different peptides or proteins.

In some embodiments, the first particles and optionally the second particles are magnetic.

In some embodiments, the buffer is Tris buffer.

In some aspects, the disclosure provides a method comprising: (a) incubating a composition in contact with one or more surfaces, wherein the composition comprises a biological sample and a buffer configured to maintain a pH of the composition between 8 and 11, wherein biomolecules from the biological sample are adsorbed onto the one or more surfaces; (b) enriching for the biomolecules that have adsorbed to the surfaces; (c) assaying the enriched biomolecules to identify one or more of the enriched biomolecules from the biological sample.

In some embodiments, the buffer is configured to maintain a pH of the composition between 9 and 10.

In some embodiments, the buffer is configured to maintain a pH of the composition of about 9.5. In some embodiments, the buffer is Tris buffer.

In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample is plasma or serum.

In some embodiments, the biological sample is cell culture media.

In some embodiments, the composition comprises no more than 60% by volume of the biological sample.

In some embodiments, the composition comprises no more than 25% by volume of the biological sample.

In some embodiments, the composition comprises no more than 15% by volume of the biological sample.

In some embodiments, the composition comprises at least 1% by volume of the biological sample.

In some embodiments, the composition comprises at least 4% by volume of the biological sample.

In some embodiments, the composition comprises at least 10% by volume of the biological sample.

In some embodiments, the composition comprises at least 20% by volume of the biological sample.

In some embodiments, the assaying comprises SDS-PAGE, gel-based separation techniques, an immunoassay, ELISA, high performance liquid chromatography, mass spectrometry, Edman Degradation, or immunoaffinity techniques.

In some embodiments, the assaying comprises mass spectrometry.

In some embodiments, the assaying comprises LC-MS/MS.

In some embodiments, the assaying comprises quantifying the biomolecules.

In some embodiments, the assaying comprises identifying one or more proteins in the biological sample.

In some embodiments, at least 100 different proteins are identified in the biological sample.

In some embodiments, at least 250 different proteins are identified in the biological sample.

In some embodiments, at least 500 different proteins are identified in the biological sample.

In some embodiments, the one or more surfaces are comprised on particles dispersed in the composition during the incubation.

In some embodiments, the nanoparticles have an average diameter of less than 500 nanometers.

In some embodiments, the particles are magnetic.

In some embodiments, the particles comprise an outer layer of poly(dimethyl aminopropyl methacrylamide) (PDMAPMA).

In some embodiments, the particles comprise an outer layer of silica.

In some embodiments, the particles comprise a carboxylate-functionalized outer layer.

In some embodiments, the particles comprise an amine-functionalized outer layer.

In some embodiments, the particles comprise a polymeric outer layer.

In some embodiments, the particles comprise a polyacrylamide outer layer.

In some embodiments, the particles comprise an acrylate polymer.

In some embodiments, the method further comprises: (a) incubating a second composition in contact with one or more second surfaces, wherein the second composition comprises the biological sample, wherein biomolecules from the biological sample are adsorbed onto the one or more second surfaces; (b) enriching for the biomolecules that have adsorbed to the one or more second surfaces; (c) assaying the enriched biomolecules to identify one or more of the enriched biomolecules from the one or more second surfaces in the biological sample.

In some embodiments, the second composition has a pH between 5 and 9.

In some embodiments, the second composition has a pH between 6 and 8.

In some embodiments, the second composition has a pH between 8 and 11.

In some embodiments, the second composition has a pH between 9 and 10.

In some embodiments, the second composition does not comprise a buffer.

In some embodiments, the second composition comprises a buffer that is different from the buffer configured to maintain a pH of the composition between 8 and 11.

In some embodiments, the second composition comprises the same buffer as the composition.

In some embodiments, the incubating of the composition and the incubating of the second composition occur at the same time.

In some embodiments, the assay identifies the enriched biomolecules with a coefficient of variance of at most 30%.

In some embodiments, the assay identifies the enriched biomolecules with a coefficient of variance of at most 25%.

In some embodiments, the method further comprises digesting proteins adsorbed to the first surface before assaying the biomolecules.

In some embodiments, the method further comprises treating proteins within the biomolecule corona with a reducing agent before assaying the biomolecules.

In some embodiments, the method further comprises treating proteins within the biomolecule corona with an alkylating agent before assaying the biomolecules.

In some aspects, the present disclosure provides a suspension comprising: (a) a first particle having a first functionalized surface chemistry; (b) a second particle having a second functionalized surface chemistry, wherein the first functionalized surface chemistry is different from the second functionalized surface chemistry of the second particle, and wherein both the first particle and the second particle have a negative charge; (c) a biological sample; and (d) a buffer configured to maintain a pH of about 8 to about 11.

In some embodiments, the buffer is configured to maintain a pH between 9 and 10.

In some embodiments, the buffer is Tris buffer having a pH between 9 and 10.

In some embodiments, the first particle is a magnetic particle and comprises an outer layer with a silanol surface functionalization, and the second particle is a magnetic particle and comprises an outer layer with a carboxylate surface functionalization.

In some embodiments, the first composition has a ratio of about 10 to about 15 parts by weight of the first particle to about 1 part by weight of the second particle.

In some embodiments, a ratio of a mean diameter for the first particle to a mean diameter of the second particle is about 3:2 to about 2:3.

In some embodiments, the first particle and the second particle are superparamagnetic iron oxide nanoparticles.

In some aspects, the present disclosure provides a suspension comprising: (a) a first particle having a first functionalized surface chemistry; (b) a second particle having a second functionalized surface chemistry, wherein the first functionalized surface chemistry is different from the second functionalized surface chemistry of the second particle, and wherein both the first particle and the second particle have a positive charge; and (c) a biological sample.

In some embodiments, the first particle is a magnetic particle and comprises an outer layer with an amine surface functionalization, and the second particle is a magnetic particle and comprises an outer layer with an amine surface functionalization.

In some embodiments, the outer layer of the first particle comprises a polymer.

In some embodiments, the suspension has a ratio of about 10 to about 15 parts by weight of the first particle to about 1 part by weight of the second particle.

In some embodiments, a ratio of a mean diameter for the first particle to a mean diameter of the second particle is about 3:2 to about 2:3.

In some embodiments, the first particle and the second particle are superparamagnetic iron oxide nanoparticles.

In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample comprises plasma or serum.

In some embodiments, the biological sample is a cell-free biological sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a method for performing an assay, in accordance with some embodiments.

FIG. 2A shows compositions of pH adjusted biological samples, in accordance with some embodiments.

FIG. 2B shows a 96 well plate layout for performing PROTEOGRAPH™ assays using pH adjusted biological samples, in accordance with some embodiments.

FIG. 2C shows compositions of pH adjusted wash buffers, in accordance with some embodiments.

FIG. 3 shows the number of protein groups detected using PROTEOGRAPH™ assays with different particles with pH adjusted biological samples of various pH, in accordance with some embodiments.

FIG. 4 shows the mass of peptides detected using PROTEOGRAPH™ assays with different particles with pH adjusted biological samples of various pH, in accordance with some embodiments.

FIG. 5 shows the miscleavage rate of proteins detected using PROTEOGRAPH™ assays with different particles with pH adjusted biological samples of various pH, in accordance with some embodiments.

FIG. 6A shows a statistical comparison of protein groups identified using PROTEOGRAPH™ assays with a pH adjusted buffer versus water, in accordance with some embodiments.

FIGS. 6B-6D show a comparison of protein groups concentrations identified using PROTEOGRAPH™ assays with a pH adjusted buffer versus water, in accordance with some embodiments.

FIG. 7A shows a comparison of functional annotations of protein groups identified using PROTEOGRAPH™ assays with a pH adjusted buffer versus water, in accordance with some embodiments.

FIGS. 7B-7D shows a comparison of functional annotations of protein groups identified using PROTEOGRAPH™ assays with a pH adjusted buffer versus water, in accordance with some embodiments.

FIG. 8A shows a statistical comparison of protein groups identified using PROTEOGRAPH™ assays with a pH adjusted buffer versus water, in accordance with some embodiments.

FIGS. 8B-8D show a comparison of protein groups concentrations identified using PROTEOGRAPH™ assays with a pH adjusted buffer versus water, in accordance with some embodiments.

FIG. 9A shows a comparison of functional annotations of protein groups identified using PROTEOGRAPH™ assays with a pH adjusted buffer versus water, in accordance with some embodiments.

FIGS. 9B-9D shows a comparison of functional annotations of protein groups identified using PROTEOGRAPH™ assays with a pH adjusted buffer versus water, in accordance with some embodiments.

FIG. 10A shows a statistical comparison of protein groups identified using PROTEOGRAPH™ assays with a pH adjusted buffer versus water, in accordance with some embodiments.

FIGS. 10B-10D show a comparison of protein groups concentrations identified using PROTEOGRAPH™ assays with a pH adjusted buffer versus water, in accordance with some embodiments.

FIG. 11A shows a comparison of functional annotations of protein groups identified using PROTEOGRAPH™ assays with a pH adjusted buffer versus water, in accordance with some embodiments.

FIGS. 11B-11D shows a comparison of functional annotations of protein groups identified using PROTEOGRAPH™ assays with a pH adjusted buffer versus water, in accordance with some embodiments.

FIG. 12 shows median protein group counts for particle panels, in accordance with some embodiments. A two particle panel uses pH 9.5 for one particle, and pH of 7 for another particle.

FIG. 13A shows a statistical comparison of protein groups identified using PROTEOGRAPH™ assays with a pH adjusted buffer versus water, in accordance with some embodiments.

FIGS. 13B-13D show a comparison of protein groups concentrations identified using PROTEOGRAPH™ assays with a pH adjusted buffer versus water, in accordance with some embodiments.

FIG. 14A shows a comparison of functional annotations of protein groups identified using PROTEOGRAPH™ assays with a pH adjusted buffer versus water, in accordance with some embodiments.

FIGS. 14B-14D shows a comparison of functional annotations of protein groups identified using PROTEOGRAPH™ assays with a pH adjusted buffer versus water, in accordance with some embodiments.

FIG. 15 shows the number of protein groups identified using PROTEOGRAPH™ assays with particle panels at different pH, in accordance with some embodiments. 17% increase in number of protein groups identified was observed using high pH.

FIG. 16 shows particle panels with mixed buffer conditions, in accordance with some embodiments.

FIGS. 17A-17F show statistical comparisons of protein groups identified using PROTEOGRAPH™ assays with a particle at different pH, in accordance with some embodiments.

FIGS. 18A-18F show statistical comparisons of protein groups identified using PROTEOGRAPH™ assays with a particle at different pH, in accordance with some embodiments.

FIGS. 19A-19F show statistical comparisons of protein groups identified using PROTEOGRAPH™ assays with a particle at different pH, in accordance with some embodiments.

FIG. 20 shows a method for corona formation, in accordance with some embodiments.

FIGS. 21A-21B shows a 96 well plate layout for performing PROTEOGRAPH™ assays using pH adjusted biological samples, in accordance with some embodiments.

FIG. 22 shows a comparison for the number of protein groups identified between a multiplexed assay compared to a single-plex assay, in accordance with some embodiments.

FIG. 23 shows a comparison for the number of protein groups identified between various multiplexed assays with different Vroman conditions, in accordance with some embodiments.

FIG. 24 shows a comparison for the number of protein groups identified between various multiplexed assays, in accordance with some embodiments.

FIG. 25 shows a comparison for the number of protein groups identified between various multiplexed assays with different pH conditions, in accordance with some embodiments.

FIGS. 26A-26I show surfaces, in accordance with some embodiments. A surface may be functionalized at one or more regions for capturing biomolecules. A surface may comprise one or more wells or depressions for capturing biomolecules. For example, a functionalized surface may be disposed in a 96 well plate or a 384 well plate. A surface may be disposed on one or more particles. In some embodiments, the one or more particles may be disposed in one or more wells or depressions. A surface may be disposed on a plurality of particles packed in a channel or a porous material disposed in a channel. A surface may be disposed on an inner surface of a channel. A surface may comprise 1, 2, 3, 4 or any number of distinct surface regions. In some cases, a surface may be disposed on a particle. In some cases, a particle may be a porous particle.

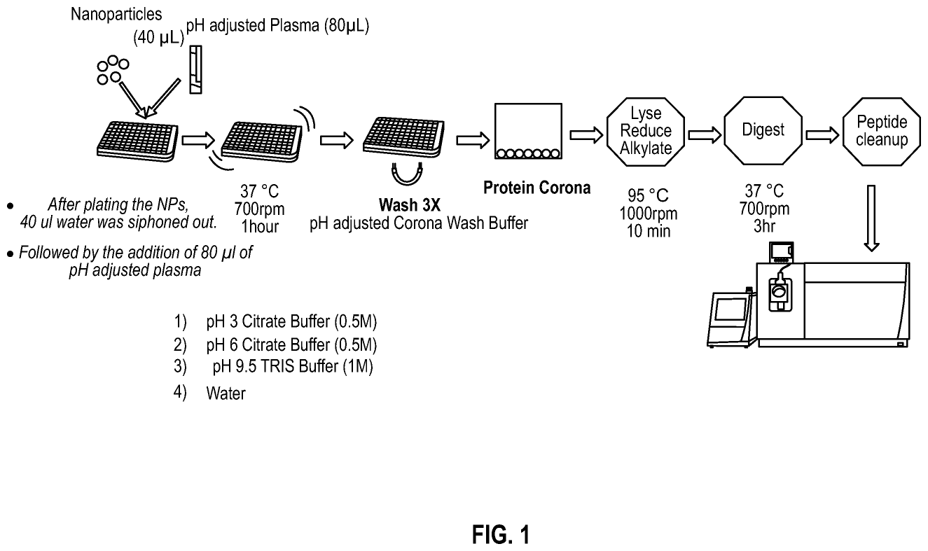

### DETAILED DESCRIPTION

Though the human genome contains about 20,000 genes, some researchers estimate that the human proteome contains over 1 million proteins expressed from those genes. A number of different proteoforms can be expressed from a repertoire of various transcriptional, translational, and post-translational mechanisms (e.g., alternative splice forms, allelic variations, and protein modifications) that produce proteins that differ from those that comprise the canonical sequence expressed from the genes. Of the vast number of proteins estimated to exist in the human proteome, only a small fraction has thus been meaningfully identified and/or quantified in the human body.

Some of the challenges in identifying and quantifying the proteins is related to the rarity of certain proteins. For instance, human plasma contains protein species over a dynamic range that exceeds 12 magnitudes, where the top few proteins (e.g., albumin, transferrin, complement proteins, apolipoproteins, and alpha-2-macroglobulin) comprise 95% of the mass of protein in the plasma, and most of the protein species comprise the remaining 5%. Some of the protein species exist in the nanograms per milliliter ranges (e.g., transforming growth factor beta-1-induced transcript 1 protein at ˜10 ng/ml; fructose-bisphosphate aldolase A at ˜20 ng/ml; thioredoxin at ˜18 ng/ml; and L-selectin at ˜92 ng/ml), and some proteins are expected to be present at levels even beneath that range. Liquid chromatography coupled with mass spectrometry (LC-MS) or tandem mass spectrometry (LC-MS/MS) has grown into ubiquitous detection platforms due to their speed, sensitivity, and breadth of applications. LC-MS and LC-MS/MS can be used to identify protein species, however, due to the stochastic nature of the methods, only a fraction of ionic species that are generated at a time from a given sample may be selected for acquiring mass spectra. As a result, the presence of species that are highly abundant compared to the rare species can create an overwhelming number of signals that make the rare species elusive.

Some aspects of the PROTEOGRAPH™ technology aims to solve some of these challenges by “compressing” the dynamic range of protein species in a sample. Some aspects of the PROTEOGRAPH™ technology operates based on non-specific binding of proteins to nanoparticle surfaces to form protein coronas. Without requiring a presence of a specific entity that is configured for binding to a singular specific protein (e.g., as in immunoassays), the non-specific binding can result in a dynamic range compression of proteins bound to the nanoparticle surfaces while capturing a wide variety of proteins. In other words, the relative abundance of proteins in the sample can be modified on the nanoparticle surfaces, such that the rare proteins are relatively more abundant, and the highly abundant proteins are relatively less abundant compared to the original sample. The proteins can then be separated from the sample and analyzed, for example, with mass spectrometry. The compressed dynamic range can allow rare proteins to comprise a higher fraction of ionic species, thereby allowing higher probability for detecting those rare proteins in a MS experiment. Though the above example is described in terms of proteins, other biomolecule classes (e.g., lipids, sugars, etc.) can be similarly targeted. Other aspects of the PROTEOGRAPH™ technology include controlled automation of the PROTEOGRAPH™ workflow that increases speed/throughput and accuracy/reliability.

While the introduction of the PROTEOGRAPH™ technology increased the number of proteins that can be detected from samples, another challenge is presented, which is to find biomarkers and/or therapeutic targets among those proteins. As the number of proteins that can be considered for diagnostic or therapeutic potential increases, the sample size may also be increased in order to effectively screen for the relevant proteins. Due to individual differences in biology between humans, thousands of proteins can have varying levels in plasma samples between two individuals. Therefore, samples from hundreds or thousands of individuals may be experimented with to identify meaningful and systematic signals that have clinical relevance. Thus, some aspects of the present disclosure provides systems, compositions, and methods, that provide higher depth (e.g., increased number of detected biomolecules and increased dynamic range) and throughput, which can identify more subtle minutiae present in biological signatures of complex biological samples. By increasing depth, more obscure biological signatures (e.g., those associated with very rare proteins) can become more visible. By increasing throughput, spurious variations in data (e.g., associated with known and unknown sources of error, i.e., standard deviation/error) can be mitigated by improving certainty.

In some aspects, the present disclosure provides a composition of multiple particles with physicochemically distinct characteristics in one volume. Physicochemically distinct particles can each enrich/deplete a different section of the biomolecule profile in biological sample. Thus, using multiple physicochemically distinct particles can provide advantages in profiling biological samples at higher dynamics ranges. By multiplexing physicochemically distinct particles together into a single volume, e.g., a well in a 96-well plate, PROTEOGRAPH™ experiments may be performed with higher throughput than, say, when the particles are each placed into separate well. As a simple illustration, multiplexing two particles in each well can improve overall throughput by a factor of two. In some aspects, the present disclosure provides systems and methods employing such compositions.

Another aspect of the present disclosure provides a composition comprising a particle and a solvent environment that is tailored to the particle. The tailored solvent environment can comprise a certain solvent, salt, pH, etc., which can improve the number of biomolecules identified in a PROTEOGRAPH™ method. Another aspect of the present disclosure provides a composition comprising multiplexed particles and a solvent environment that is tailored to the multiplexed particles. The tailored solvent environment can increase the number of identified biomolecules, but can also improve the stability and/or dispersibility of the multiplexed particles. In some aspects, the present disclosure provides systems and methods employing such compositions.

Another aspect of the present disclosure provides methods and systems for performing fast, scalable, deep, and unbiased plasma proteomics. In some cases, the methods and systems may be used to identify known and/or novel biomarkers for diseases. In some cases, the methods and systems may be used to facilitate identification of disease-relevant protein variants, for instance, as disclosed in PCT/US2023/060271, which is incorporated by reference in its entirety herein. Important advances in characterizing the proteomic landscape of lung cancers such as non-small cell lung cancer (NSCLC) and squamous cell lung cancer have identified important protein biomarkers. However, relatively few proteoforms relevant to lung cancer have been identified. Readout technologies such as high resolution quantitative mass spectrometry (MS) can be employed to infer and to quantify peptides and proteins with high confidence (e.g., <1% false discovery rate (FDR)). However, large-scale LC-MS/MS-based proteomics studies can be challenging due to lengthy workflows required to achieve deep (e.g., broad detection of proteins across the dynamic range, from high to low abundance proteins) and unbiased (e.g., hypothesis-free detection) sampling of clinically relevant biospecimens with large dynamic ranges of protein abundances, such as blood plasma. While LC-MS and LC-MS/MS methodologies may offer the capability to infer proteoforms, peptide identification in LC-MS/MS-based proteomic data may rely on protein databases, such as UniProt, which may exclude proteoforms that may be present in an individual's proteome. In some cases, the compositions, methods, and systems disclosed herein may be used to observe examples of alternative exon usage. In some cases, the compositions, methods, and systems disclosed herein may be used to identify proteoforms arising from alternative splicing. In some cases, the compositions, methods, and systems disclosed herein may be used to identify proteoforms arising from genetic variation. In some cases, the compositions, methods, and systems disclosed herein may be used to identify proteoforms based at least partially on custom protein databases generated from subject-matched genotype data, such as whole exome sequencing (WES) data. In some cases, the compositions, methods, and systems disclosed herein may be used to discover new proteoforms. In some cases, the compositions, methods, and systems disclosed herein may be used to identify proteoforms that would otherwise not be identified using protein affinity-based targeted technologies. In some cases, the compositions, methods, and systems disclosed herein may be used to support enhanced understanding of human health and disease by identifying proteoforms.

In some aspects, the present disclosure provides a method for selectively enriching and assaying a plurality of biomolecules in a fluid composition. In some embodiments, the method comprises selectively enriching a first plurality of biomolecule types in a first fluid composition. In some embodiments, the method comprises selectively enriching a second plurality of biomolecule types in a second fluid composition. In some embodiments, the method comprises performing a downstream assay on biomolecule types of the first plurality of biomolecule types and the second plurality of biomolecule types.

The methods, compositions, and systems disclosed herein may, for example, provide for improved proteomic analysis of biological samples. In some embodiments, similar or more protein groups can be detected in biological samples using less mass spectrometry injections and/or shorter chromotography gradients in LC-MS/MS. For example, similar protein groups may be identified using two 30-minute gradient injections comparted to five 30-minute gradient injections. In some embodiments, less sample volume may be used while still obtaining similar protein group identifications.

### Methods for Selectively Enriching and Assaying Biomolecules

In some embodiments, the selectively enriching the first plurality of biomolecule types in the fluid composition comprises contacting the first fluid composition with a first surface to adsorb the first plurality of biomolecule types on the first surface. In some embodiments, the selectively enriching the second plurality of biomolecule types in the fluid composition comprises contacting the second fluid composition with a second surface to adsorb the second plurality of biomolecule types on the second surface. In some embodiments, the selectively enriching the first plurality of biomolecule types in the fluid composition comprises contacting the first fluid composition with a first plurality of surface types to adsorb the first plurality of biomolecule types on the first plurality of surface types. In some embodiments, the selectively enriching the second plurality of biomolecule types in the fluid composition comprises contacting the second fluid composition with a second plurality of surface types to adsorb the second plurality of biomolecule types on the second plurality of surface types.

In some embodiments, the first plurality of surface types and the second plurality of surface types comprise the same sign of charge. In some embodiments, the first plurality of surface types and the second plurality of surface types comprise a different sign of charge. In some embodiments, the first plurality of surface types and the second plurality of surface types comprise the same sign of zeta potential. In some embodiments, the first plurality of surface types and the second plurality of surface types comprise a different sign of zeta potential. In some embodiments, the first plurality of surface types, the second plurality of surface types, or both comprise an acidic functional group. In some embodiments, the acidic functional group comprises a Bronsted-Lowry acid or a Lewis acid functional group. In some embodiments, the first plurality of surface types, the second plurality of surface types, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof. In some embodiments, the first plurality of surface types, the second plurality of surface types, or both comprise a basic functional group. In some embodiments, the basic functional group comprises a Bronsted-Lowry acid or a Lewis acid functional group.

In some embodiments, the first fluid composition is incubated with the first surface for at least 20 minutes at at least 30° C. to adsorb the first plurality of biomolecule types on the first surface. In some embodiments, the second fluid composition is incubated with the second surface for at least 20 minutes at at least 30° C. to adsorb the second plurality of biomolecule types on the second surface. In some embodiments, the first fluid composition is incubated with the first surface for at least 20 minutes at at least 30° C. to adsorb the first plurality of biomolecule types on the first surface, and the second fluid composition is incubated with the second surface for at least 20 minutes at at least 30° C. to adsorb the second plurality of biomolecule types on the second surface.

In some embodiments, the first plurality of biomolecule types is depleted in at least a first biomolecule and enriched in a second biomolecule, wherein the first biomolecule is more abundant than the second biomolecule in the first fluid composition. In some embodiments, the second plurality of biomolecule types is depleted in at least a first biomolecule and enriched in a second biomolecule, wherein the first biomolecule is more abundant than the second biomolecule in the second fluid composition. For example, the first fluid composition and the second fluid composition may each comprise the same particle composition, but different pH, solvent, temperature, pressure, and the like as described herein. Even though the particle compositions are the same between the first fluid composition and the second fluid composition, they may enrich and deplete different biomolecules. In some cases, the first and the second particle compositions can be different.

In some embodiments, the first surface is disposed in a first lyophilized composition comprising (i) at least one nanoparticle, and (ii) one or more modifiers. In some embodiments, the second surface is disposed in a second lyophilized composition comprising (i) at least one nanoparticle, and (ii) one or more modifiers. In some embodiments, the one or more modifiers comprise pH modifiers, ionic strength modifiers, viscosity modifiers, or any combination thereof. In some embodiments, a surface, upon reconstitution of the lyophilized composition in a solution, has a mean zeta potential that is between 85% to 115% of the zeta potential of a same particle dissolved in a same solution in the absence of lyophilization, as determined by zeta potential measurements.

In some embodiments, a surface, upon reconstitution of the lyophilized composition in a solution, has a mean zeta potential that is between 90% to 110% of the zeta potential of a same particle dissolved in a same solution in the absence of lyophilization, as determined by zeta potential measurements. In some embodiments, a surface, upon reconstitution of the lyophilized composition in a solution, has a mean zeta potential that is between 95% to 105% of the zeta potential of a same particle dissolved in a same solution in the absence of lyophilization, as determined by zeta potential measurements. In some embodiments, a surface, upon reconstitution of the lyophilized composition in a solution, has a mean zeta potential standard deviation that is between 85% to 115% of the zeta potential standard deviation of a same particle dissolved in a same solution in the absence of lyophilization, as determined by zeta potential measurements. In some embodiments, a surface, upon reconstitution of the lyophilized composition in a solution, the particle has a mean zeta potential standard deviation that is between 90% to 110% of the zeta potential standard deviation of a same particle dissolved in a same solution in the absence of lyophilization, as determined by zeta potential measurements. In some embodiments, a surface, upon reconstitution of the lyophilized composition in a solution, the particle has a zeta potential standard deviation that is between 95% to 105% of the zeta potential standard deviation of a same particle dissolved in a same solution in the absence of lyophilization, as determined by zeta potential measurements. In some embodiments, a surface, upon reconstitution of the lyophilized composition in a solution, the particle has a mean diameter that is between 85% to 115% of the mean diameter of a same particle dissolved in a same solution in the absence of lyophilization, as determined by dynamic light scattering (DLS). In some embodiments, a surface, upon reconstitution of the lyophilized composition in a solution, the particle has a mean diameter that is between 90% to 110% of the mean diameter of a same particle dissolved in a same solution in the absence of lyophilization, as determined by dynamic light scattering (DLS). In some embodiments, a surface, upon reconstitution of the lyophilized composition in a solution, the particle has a mean diameter that is between 95% to 105% of the mean diameter of a same particle dissolved in a same solution in the absence of lyophilization, as determined by dynamic light scattering (DLS). In some embodiments, a surface, upon reconstitution of the lyophilized composition in a solution, the particle has a mean diameter that is between 98% to 102% of the mean diameter of a same particle dissolved in a same solution in the absence of lyophilization, as determined by dynamic light scattering (DLS). In some embodiments, a surface, upon reconstitution of the lyophilized composition in a solution, the particle has a diameter standard deviation that is between 85% to 115% of the diameter standard deviation of a same particle dissolved in a same solution in the absence of lyophilization, as determined by dynamic light scattering (DLS). In some embodiments, a surface, upon reconstitution of the lyophilized composition in a solution, the particle has a diameter standard deviation that is between 90% to 110% of the diameter standard deviation of a same particle dissolved in a same solution in the absence of lyophilization, as determined by dynamic light scattering (DLS). In some embodiments, a surface, upon reconstitution of the lyophilized composition in a solution, the particle has a diameter standard deviation that is between 95% to 105% of the diameter standard deviation of a same particle dissolved in a same solution in the absence of lyophilization, as determined by dynamic light scattering (DLS). In some embodiments, a surface, upon reconstitution of the lyophilized composition in a solution, the particle has a diameter standard deviation that is between 98% to 102% of the diameter standard deviation of a same particle dissolved in a same solution in the absence of lyophilization, as determined by dynamic light scattering (DLS). Lyophilized compositions may have stable physicochemical properties over various periods of time. In some cases, the period of time may comprise a period of at least about 12 days, at least about 14 days, at least about 30 days, at least 40 days, at least about 2 months, at least about 3 months, at least about 4 months, at least about 5 months, at least about 6 months, at least about 7 months, at least about 8 months, at least about 9 months, at least about 10 months, at least about 11 months, or at least about 1 year. Lyophilized compositions may have stable physicochemical properties at various temperatures. In some cases, the temperature may be about room temperature. In some cases, the temperature may be about 37° C. In some cases, the temperature may be about 60° C. In some cases, the temperature may be about −26° C. to about 0° C. In some cases, the temperature may be about −10° C. to about −5° C. In some cases, the temperature may be about 0° C. to 20° C. In some cases, the temperature may be about 0° C. to about 10° C. In some cases, the temperature may be about 25° C. to about 60° C. In some cases, the temperature may be about 35° C. to about 40° C. In some cases, the dry composition or lyophilized composition is stable at about 37° C. for at least 40 days. In some cases, the dry composition or lyophilized composition is stable at ambient temperature for at least 11 months.

In some embodiments, a surface is comprised on a particle dispersed in the composition during incubation. In some embodiments, a particle is a nanoparticle. In some embodiments, nanoparticles have an average diameter of less than 500 nanometers. In some embodiments, a particle is a porous particle. In some embodiments, a particle is a microparticle. In some embodiments, a particle comprises a paramagnetic material. In some embodiments, a paramagnetic material is a superparamagnetic material. In some embodiments, a paramagnetic material comprises iron oxide, aluminum, platinum, or any combination thereof. In some embodiments, a particle comprises an outer layer of poly(dimethyl aminopropyl methacrylamide) (PDMAPMA). In some embodiments, a particle comprises an outer layer of silica. In some embodiments, a particle comprises a carboxylate-functionalized outer layer. In some embodiments, a particle comprises an amine-functionalized outer layer. In some embodiments, a particle comprises a polymeric outer layer. In some embodiments, a particle comprises a polyacrylamide outer layer. In some embodiments, a particle comprises an acrylate polymer.

In some embodiments, the method further comprises digesting proteins adsorbed to the first surfaces before assaying the biomolecules. In some embodiments, the method further comprises treating proteins within the biomolecule corona with a reducing agent before assaying the biomolecules. In some embodiments, the method further comprises treating proteins within the biomolecule corona with an alkylating agent before assaying the biomolecules.

In some embodiments, the method further comprises washing the first plurality of biomolecule types with a first wash composition and washing the second plurality of biomolecule types with a second wash composition. In some embodiments, the first fluid composition and the first wash composition comprise at least one common intensive physical property. In some embodiments, the first fluid composition and the first wash composition comprise at least one different intensive physical property. In some embodiments, the first fluid composition and the first wash composition comprise at least one common solvent. In some embodiments, the first fluid composition and the first wash composition comprise at least one different solvent. In some embodiments, the first fluid composition and the first wash composition comprise about the same ionic strength. In some embodiments, the first fluid composition and the first wash composition comprise different ionic strengths. In some embodiments, the first fluid composition and the first wash composition comprise at least one common salt. In some embodiments, the first fluid composition and the first wash composition comprise at least one different salt. In some embodiments, the first fluid composition and the first wash composition comprise about the same pH. In some embodiments, the first fluid composition and the first wash composition comprise different pH.

In some embodiments, the second fluid composition and the second wash composition comprise at least one common intensive physical property. In some embodiments, the second fluid composition and the second wash composition comprise at least one different intensive physical property. In some embodiments, the second fluid composition and the second wash composition comprise at least one common solvent. In some embodiments, the second fluid composition and the second wash composition comprise at least one different solvent. In some embodiments, the second fluid composition and the second wash composition comprise about the same ionic strength. In some embodiments, the second fluid composition and the second wash composition comprise different ionic strengths. In some embodiments, the second fluid composition and the second wash composition comprise at least one common salt. In some embodiments, the second fluid composition and the second wash composition comprise at least one different salt.

In some embodiments, the first wash composition and the second wash composition comprise at least one common intensive physical property. In some embodiments, the first wash composition and the second wash composition comprise at least one different intensive physical property. In some embodiments, the first wash composition and the second wash composition comprise about the same ionic strength. In some embodiments, the first wash composition and the second wash composition comprise different ionic strengths. In some embodiments, the first wash composition and the second wash composition comprise at least one common salt. In some embodiments, the first wash composition and the second wash composition comprise at least one different salt. In some embodiments, the first wash composition and the second wash composition comprise about the same pH. In some embodiments, the first wash composition and the second wash composition comprise different pH. In some embodiments, the first wash composition and the second wash composition are the same. In some embodiments, the first wash composition and the second wash composition are different. Various intensive properties, solvents, salts, and pH have been described elsewhere herein.

In some embodiments, the first wash composition releases the first plurality of biomolecule types adsorbed on the first surface from the first surface. In some embodiments, the method further comprises purifying the first plurality of biomolecule types to produce a first purified composition. In some embodiments, the purifying comprises drying the plurality of biomolecule types to remove the first wash composition. In some embodiments, the method further comprises reconstituting the first purified composition with a first reconstitution composition to produce a first reconstituted composition. In some embodiments, the second wash composition releases the second plurality of biomolecule types deposited on the second surface from the second surface. In some embodiments, the method further comprises purifying the second plurality of biomolecule types to produce a second purified composition. In some embodiments, the purifying comprises drying the second plurality of biomolecule types to remove the second wash composition. In some embodiments, the method further comprises reconstituting the second purified composition with a second reconstitution composition to produce a second reconstituted composition.

The first or the second plurality of biomolecule types can comprise peptides. The peptides can comprise proteolytically cleaved derivatives of proteins adsorbed on the first or the second surfaces. The peptides could have been proteolytically cleaved by a protease, e.g., trypsin or lysin. The drying can comprise applying negative pressure (e.g., negative gauge pressure with respect to atmospheric pressure), while optionally heating or chilling the drying sample. The drying may proceed to the extent until solvent evaporation is no longer observable, e.g., through changes in mass. The drying can be performed for at least 1, 2, 3, 4, 5, 6, 7, 8, 9, 10, 20, 30, 40, 50, or 60 minutes. The drying can be performed for at least 1, 2, 3, 4, 5, 6, 7, 8, 9, 11, 12, 24, 36, or 48 hours. The drying can be performed for at most 1, 2, 3, 4, 5, 6, 7, 8, 9, 10, 20, 30, 40, 50, or 60 minutes. The drying can be performed for at most 1, 2, 3, 4, 5, 6, 7, 8, 9, 11, 12, 24, 36, or 48 hours. The drying can be performed at about room temperature. The drying can be performed at a temperature of at least −200, −150, −100, −50, −25, 0, 25, 50, 75, or 100° C. The drying can be performed at a temperature of at most −200, −150, −100, −50, −25, 0, 25, 50, 75, or 100° C. The drying can be performed at a negative gauge pressure of at least 10, 20, 30, 40, 50, 60, 70, 80, 90, or 100 kilopascals (kPa). The drying can be performed at a negative gauge pressure of at most 10, 20, 30, 40, 50, 60, 70, 80, 90, or 100 kPa. Reconstituting can be performed by adding a buffer. Various buffer compositions have been described elsewhere herein. The first reconstitution composition and the second reconstitution composition can be the same or different. The first biological sample and the second biological sample, when reconstituted, can comprise about a predetermined concentration of peptides. The predetermined concentration can be at least about 1, 2, 3, 4, 5, 6, 7, 8, 9, 10, 20, 30, 40, 50, 60, 70, 80, 90, 100, 200, 300, 400, 500, 600, 700, 800, 900, or 1,000 ng/μL (biomolecule mass/buffer volume). The predetermined concentration can be at most about 1, 2, 3, 4, 5, 6, 7, 8, 9, 10, 20, 30, 40, 50, 60, 70, 80, 90, 100, 200, 300, 400, 500, 600, 700, 800, 900, or 1,000 ng/μL (biomolecule mass/buffer volume).

Various analytical methods for identifying peptide or protein species can be used in assaying (e.g., downstream assay). In some embodiments, assaying comprises SDS-PAGE, gel-based separation techniques, an immunoassay, ELISA, high performance liquid chromatography, mass spectrometry, Edman Degradation, or immunoaffinity techniques. In some embodiments, assaying comprises mass spectrometry. In some embodiments, assaying comprises LC-MS/MS. In some embodiments, assaying comprises quantifying the biomolecules. In some embodiments, assaying comprises identifying one or more proteins in the biological sample. In some embodiments, the downstream assay comprises mass spectrometry. In some embodiments, the downstream assay comprises protein sequencing. In some embodiments, the downstream assay comprises LC-MS/MS. In some embodiments, the downstream assay comprises an immunoassay. In some embodiments, the downstream assay comprises contacting the first plurality of biomolecule types, the second plurality of biomolecule types, or both with a pair of antibodies capable of binding to at least one biomolecule type in the first plurality of biomolecule types, the second plurality of biomolecule types, or both, wherein the pair of antibodies comprises complementary single-stranded nucleic acid sequences attached thereto, such that when the pair of antibodies bind to the at least one biomolecule type, the complementary nucleic acids hybridize to form a double stranded nucleic acid, wherein the double stranded nucleic acid is configured to form a binding complex with a polymerase and a plurality of nucleotides, nucleosides, nucleotide analogs, and/or nucleoside analogs to perform an amplification reaction to produce a detectable signal. In some embodiments, the downstream assay comprises contacting the first plurality of biomolecule types, the second plurality of biomolecule types, or both with one or more aptamers capable of binding to at least one biomolecule type in the first plurality of biomolecule types, the second plurality of biomolecule types, or both, wherein the one or more aptamers are coupled to a surface via a cleavable linker. In some embodiments, the cleavable linker is photocleavable. In some embodiments, the surface is a particle surface. In some embodiments, the method further comprises contacting the first plurality of biomolecule types, the second plurality of biomolecule types, or both with a macromolecular competitor configured to, in a fluid composition, reduce dissociation of a complex comprising the one or more aptamers and the first plurality of biomolecule types, the second plurality of biomolecule types, or both. In some embodiments, the macromolecular competitor is further configured to bind to a biomolecule type that is different from the first plurality of biomolecule types, the second plurality of biomolecule types, or both. In some embodiments, the macromolecular competitor is a polyanionic macromolecule. In some embodiments, the macromolecular competitor is a polycationic macromolecule. In some embodiments, the downstream assay comprises nucleic acid sequencing.

In some embodiments, the assaying is performed at a rate of at least 1, 2, 3, 4, 5, 6, 7, 8, 9, 10, or 100 samples assayed per hour. In some embodiments, the assaying is performed at a rate of at least 1, 10, 100, 1,000, 10,000, 100,000, 1,000,000, or 10,000,000 μg of biomolecules assayed per hour. In some embodiments, the assaying is performed at a rate of at least 1, 10, 100, 1,000, 10,000, 100,000, or 1,000,000 biomolecules identified per hour. In some embodiments, the assaying is performed at a rate of at least 1, 10, 100, 1,000, 10,000, 100,000, or 1,000,000 protein groups identified per hour. The throughput of the assay can be measured with a standard, e.g., HeLa cell extract. For example, in some embodiments, the assaying identifies at least about 100, 1,000, 10,000, or 100,000 biomolecules when the suspension comprises a HeLa cell extract. In some embodiments, the plurality of biomolecules comprises at least about 6, 7, 8, 9, 10, 11, 12, 13, 14, 15, or 16 dynamic range when the assaying is performed on the plurality of biomolecules in a biological sample in the absence of the first particle and the second particle.

In some embodiments, a first likelihood that the assaying identifies a low abundance biomolecule in the plurality of biomolecules is larger than a second likelihood that the assaying identifies the low abundance biomolecule in a biological sample in the absence of the first particle and the second particle, wherein the low abundance biomolecule comprises less than about 1, 10⁻¹, 10⁻², 10⁻³, 10⁻⁴, 10⁻⁵, 10⁻⁶, 10⁻⁷, 10⁻⁸, 10⁻⁹, or 10⁻¹⁰ percent by mass of the first set of biomolecules or the second set of biomolecules. In some embodiments, the first likelihood is larger than the second likelihood by a factor of at least 2, 5, 10, 102, 102, 103, 104, 105, 106, 107, 108, 109, or 1010. In some embodiments, the assaying identifies one or more biomolecules in the plurality of biomolecules with a coefficient of variance of at most about 30, 25, 20, 15, 10, 5, 4, 3, 2, or 1 percent. In some embodiments, the assaying identifies one or more biomolecules in the plurality of biomolecules with a coefficient of variance of at least about 30, 25, 20, 15, 10, 5, 4, 3, 2, or 1 percent.

In some embodiments, the method comprises forming a first suspension comprising a first plurality of particles with a first portion of a biological sample. In some embodiments, the first plurality of particles comprises a first particle and a second particle. In some embodiments, the first particle has a first functionalized surface chemistry that is different from a second functionalized surface chemistry of the second particle. In some embodiments, the first particle has a first functionalized surface chemistry that is the same as the second functionalized surface chemistry of the second particle. In some embodiments, the both the first particle and the second particle have a negative charge. In some embodiments, the both the first particle and the second particle have a positive charge. In some embodiments, the both the first particle and the second particle have about neutral charge. In some embodiments, the both the first particle and the second particle have about zwitterionic charge. In some embodiments, the method comprises forming a second suspension comprising a second plurality of particles with a second portion of the biological sample. In some embodiments, the second plurality of particles comprises a third particle and fourth particle. In some embodiments, the third particle has a third functionalized surface chemistry that is different from a fourth functionalized surface chemistry of the fourth particle. In some embodiments, the third particle has a third functionalized surface chemistry that is the same as the fourth functionalized surface chemistry of the fourth particle. In some embodiments, the third particle and the fourth particle have a positive charge. In some embodiments, the third particle and the fourth particle have a negative charge. In some embodiments, the third particle and the fourth particle about neutral charge. In some embodiments, the third particle and the fourth particle zwitterionic charge. In some embodiments, the method comprises enriching for biomolecules that have adsorbed to the first plurality of particles. In some embodiments, the method comprises enriching for biomolecules that have adsorbed to the second plurality of parties. In some embodiments, the method comprises performing a downstream assay on the enriched biomolecules.

In some embodiments, the first suspension and the second suspension each have a pH between 9 and 10. In some embodiments, the first suspension and the second suspension each comprise Tris buffer. In some embodiments, the first suspension has a pH between 9 and 10. In some embodiments, the first suspension comprises Tris buffer. In some embodiments, the second suspension has a pH between 6 and 8. The first suspension and the second suspension can have the same or different compositions and/or properties. Various compositional parameters (e.g., buffers, salts, pH, etc.) for have been described elsewhere herein. The suspensions described herein can comprise the various compositions described elsewhere herein.

In some embodiments, the first particle is a magnetic particle. In some embodiments, the first particle comprises an outer layer with a silanol surface functionalization. In some embodiments, the second particle is a magnetic particle. In some embodiments, the second particle comprises an outer layer with a carboxylate surface functionalization. In some embodiments, the third particle is a magnetic particle. In some embodiments, the third particle comprises an outer layer with an amine surface functionalization. In some embodiments, the fourth particle is a magnetic particle. In some embodiments, the fourth particle comprises an outer layer with an amine surface functionalization. In some embodiments, the outer layer of the third particle comprises a polymer. In some embodiments, the first plurality of particles has a ratio of about 10 to about 15 parts by weight of the first particle to about 1 part by weight of the second particle. In some embodiments, the second plurality of particles has a ratio about 10 to about 15 parts by weight of the third particle to about 1 part by weight of the fourth particle. In some embodiments, a ratio of a mean diameter for the first particle to a mean diameter of the second particle is about 3:2 to about 2:3 In some embodiments, a ratio of a mean diameter for the third particle to a mean diameter of the fourth particle is about 3:2 to about 2:3. In some embodiments, each of the first particle, the second particle, the third particle, and the fourth particle are superparamagnetic iron oxide nanoparticles. Various particle compositions have been described elsewhere herein. The first particle, the second particle, the third particle, and the fourth particle can comprise various particles described herein at various ratios.

### Systems for Selectively Enriching and Assaying Biomolecules

Various parallel or sequential schemes may be used on a PROTEOGRAPH™ system to increase throughput. In some embodiments, the first fluid composition and the second fluid composition are processed on one machine. In some embodiments, the first fluid composition and the second fluid composition are processed on different machines. In some embodiments, the first fluid composition and the second fluid composition are processed in parallel. In some embodiments, the first fluid composition and the second fluid composition are performed in series. In some embodiments, the first fluid composition and the second fluid composition are a portion of the same sample. In some embodiments, the first fluid composition and the second fluid composition are from different samples. In some embodiments, the selectively enriching is performed for the first fluid composition and the second fluid composition in parallel. In some embodiments, the selectively enriching is performed for the first fluid composition and the second fluid composition in serial. In some embodiments, the selectively enriching is performed at a rate of at least 1, 10, 100, 1,000, 10,000, 100,000, 1,000,000, or 10,000,000 μg of biomolecules enriched per hour. In some embodiments, the contacting is performed for the first fluid composition and the second fluid composition in parallel. In some embodiments, the contacting is performed for the first fluid composition and the second fluid composition in serial. In some embodiments, the contacting is performed at a rate of at least 100, 1,000, 10,000, 100,000, or 1,000,000 biomolecules deposited per hour. In some embodiments, the contacting is performed at a rate of at least 1, 10, 100, 1,000, 10,000, 100,000, 1,000,000, or 10,000,000 μg of biomolecules deposited per hour. In some embodiments, the washing is performed for the first fluid composition and the second fluid composition in parallel. In some embodiments, the washing is performed for the first fluid composition and the second fluid composition in serial. In some embodiments, the washing is performed at a rate of at least 100, 1,000, 10,000, 100,000, or 1,000,000 biomolecules washed per hour. In some embodiments, the washing is performed at a rate of at least 1, 10, 100, 1,000, 10,000, 100,000, 1,000,000, or 10,000,000 μg of biomolecules washed per hour. In some embodiments, the purifying is performed for the first fluid composition and the second fluid composition in parallel. In some embodiments, the purifying is performed for the first fluid composition and the second fluid composition in serial. In some embodiments, the purifying is performed at a rate of at least 100, 1,000, 10,000, 100,000, or 1,000,000 purified per hour. In some embodiments, the purifying is performed at a rate of at least 1, 10, 100, 1,000, 10,000, 100,000, 1,000,000, or 10,000,000 μg of biomolecules purified per hour. In some embodiments, the reconstituting is performed for the first fluid composition and the second fluid composition in parallel. In some embodiments, the reconstituting is performed for the first fluid composition and the second fluid composition in serial. In some embodiments, the reconstituting is performed at a rate of at least 100, 1,000, 10,000, 100,000, or 1,000,000 reconstituted per hour. In some embodiments, the reconstituting is performed at a rate of at least 1, 10, 100, 1,000, 10,000, 100,000, 1,000,000, or 10,000,000 μg of biomolecules reconstituted per hour. In some embodiments, the downstream assay is performed for the first fluid composition and the second fluid composition in parallel. In some embodiments, the downstream assay is performed for the first fluid composition and the second fluid composition in serial. In some embodiments, the downstream assay is performed at a rate of at least 1, 2, 3, 4, 5, 6, 7, 8, 9, 10, 100 samples assayed per hour. In some embodiments, the downstream assay is performed at a rate of at least 1, 10, 100, 1,000, 10,000, 100,000, 1,000,000, or 10,000,000 μg of biomolecules assayed per hour. In some embodiments, the downstream assay is performed at a rate of at least 100, 1,000, 10,000, 100,000, or 1,000,000 biomolecules identified per hour. In some embodiments, the downstream assay is performed at a rate of at least 100, 1,000, 10,000, 100,000, or 1,000,000 protein groups identified per hour. In some embodiments, the method is performed for the first fluid composition and the second fluid composition in parallel. In some embodiments, the method is performed for the first fluid composition and the second fluid composition in serial. In some embodiments, the method is performed at a rate of at least 1, 2, 3, 4, 5, 6, 7, 8, 9, 10, 100 samples per hour. In some embodiments, the method is performed at a rate of at least 1, 10, 100, 1,000, 10,000, 100,000, 1,000,000, or 10,000,000 μg of biomolecules assayed per hour. In some embodiments, the method is performed at a rate of at least 100, 1,000, 10,000, 100,000, or 1,000,000 biomolecules identified per hour. In some embodiments, the method is performed at a rate of at least 100, 1,000, 10,000, 100,000, or 1,000,000 protein groups identified per hour. The rates of biomolecule identification can be measured using an appropriate standard. In some embodiments, the downstream assay identifies at least about 100, 1,000, 10,000, 100,000, or 1,000,000 biomolecules when the first fluid composition and the second fluid composition is a HeLa cell extract.

In some embodiments, the downstream assay identifies one or more biomolecules in the first plurality of biomolecule types, the second plurality of biomolecule types, or both, with a coefficient of variance of at most about 30, 25, 20, 15, 10, 5, 4, 3, 2, or 1 percent. In some embodiments, the downstream assay identifies one or more biomolecules in the first plurality of biomolecule types, the second plurality of biomolecule types, or both, with a coefficient of variance of at least about 30, 25, 20, 15, 10, 5, 4, 3, 2, or 1 percent. In some embodiments, the assay identifies the enriched biomolecules with a coefficient of variance of at most 30%. In some embodiments, the assay identifies the enriched biomolecules with a coefficient of variance of at most 25%. In some embodiments, the assay identifies the enriched biomolecules with a coefficient of variance of at least 0%.

In some embodiments, the first plurality of biomolecule types and the second plurality of biomolecule types together comprise at least about 6, 7, 8, 9, 10, 11, 12, 13, 14, 15, or 16 dynamic range when the downstream assay is performed on the first fluid composition and the second fluid composition before the selectively enriching in (a) and (b). In some embodiments, the first plurality of biomolecule types and the second plurality of biomolecule types together comprise at most about 6, 7, 8, 9, 10, 11, 12, 13, 14, 15, or 16 dynamic range when the downstream assay is performed on the first fluid composition and the second fluid composition before the selectively enriching in (a) and (b).

In some embodiments, a first likelihood that the downstream assay identifies a low abundance biomolecule in the first plurality of biomolecule types or the second plurality of biomolecule types is larger than a second likelihood that the downstream assay identifies the low abundance biomolecule in the first fluid composition or the second fluid composition, wherein the low abundance biomolecule comprises less than about 1, 10⁻¹, 10⁻², 10⁻³, 10⁻⁴, 10⁻⁵, 10⁻⁶, 10⁻⁷, 10⁻⁸, 10⁻⁹, or 10⁻¹⁰ percent by mass of the first set of biomolecules or the second set of biomolecules. In some embodiments, the first likelihood is larger than the second likelihood by a factor of at least 2, 5, 10, 102, 102, 103, 104, 105, 106, 107, 108, 109, or 1010.

In some embodiments, at least 250 different biomolecules are identified in the biological sample. In some embodiments, at least 500 different biomolecules are identified in the biological sample. The biomolecules can be proteins. In some embodiments, the one or more biomolecules comprise at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 peptides. In some embodiments, the one or more biomolecules comprise at most about 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 peptides. In some embodiments, the one or more biomolecules comprise at least about 100, 200, 300, 400, 500, 600, 700, 800, 900, 1,000, 2,000, 3,000, 4,000, 5,000, 6,000, 7,000, 8,000, 9,000, or 10,000 proteins. In some embodiments, the one or more biomolecules comprise at most about 100, 200, 300, 400, 500, 600, 700, 800, 900, 1,000, 2,000, 3,000, 4,000, 5,000, 6,000, 7,000, 8,000, 9,000, or 10,000 proteins.

### Compositions and Kits for Selectively Enriching and Assaying Biomolecules

In some aspects, the present disclosure provides a suspension for assaying biomolecules from a biological sample. The suspension can comprise a first particle comprising (i) a first paramagnetic portion and (ii) a first surface chemistry. The suspension can comprise a second particle comprising (i) a second paramagnetic portion and (ii) a second surface chemistry. The second surface chemistry and the first surface chemistry can be different. A plurality of biomolecules can be adsorbed on the first particle and the second particle. The suspension can be a multiplex of physicochemically distinct particles in provided in one continuous phase of solution, e.g., in a well.

The physicochemical properties of the first particle and a second particle can be such that they are colloidally stable or semi-stable in the suspension. The physicochemical properties of the first particle and a second particle can be such that they are dispersed in the suspension. The physicochemical properties of the first particle and a second particle can be such that they can be dispersed in the suspension upon agitation. The agitation can be, e.g., magnetic agitation of the particles when they are magnetic particles. Without being bound to a particular theory, the stability of a colloidal suspension can be explained in part by the Derjaguin-Landau-Verwey-Overbeek (DLVO) theory. The theory suggests that the stability of a colloidal suspension depends at least on the Bjerrum length, the Debye-Hückel screening length, the charges of the particles, and the sizes of the particles. It is worth noting that the DLVO theory does not account for all possible forces that affect colloidal stability, e.g., depletion forces are not accounted for in the theory. The Bjerrum length is the distance at which the electrostatic interaction and thermal energy are comparable. The Debye-Hückel screening length is a measure of the extent to which electrostatic fields propagate through a medium, and which in electrolyte solutions, depends at least on the dielectric constant and the ionic strength of the solution. Thus, the solvent environment of the suspension can define and/or influence the stability of the suspension, in addition to the charges and the sizes of the particles. The first particle and the second particle can comprise the same sign of charge. The charge can be, e.g., the zeta potential of the particles. The same sign of charge can be negative, positive, or about neutral. The suspension can be stable for at least about 1, 5, 10, 15, 30, or 60 minutes. The suspension can be stable for at most about 1, 5, 10, 15, 30, or 60 minutes. A time constant for destabilization of the suspension is at least about 1, 5, 10, 15, 30, or 60 minutes. A time constant for destabilization of the suspension is at most about 1, 5, 10, 15, 30, or 60 minutes. A mean aggregation number of the first particle and the second particle in the suspension can be at most about 1, 10, or 100. A mean aggregation number of the first particle and the second particle in the suspension can be at most about 1,000, 100, or 10. The suspension can comprise at least about 3, 4, 5, 6, 7, 8, 9, or 10 distinct particles. In some embodiments, the suspension is incubated at a temperature of at least 30° C.

The first surface chemistry, the second surface chemistry, or both can comprise an acidic functional group. The acidic functional group comprises a Bronsted-Lowry acid or a Lewis acid functional group. The first surface chemistry, the second first surface chemistry, or both can comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

The suspension can comprise at least about 1, 2, 3, 4, 5, 6, 7, 8, 9, or 10 parts of the first particle to about 1 part of the second particle. The suspension can comprise at most about 1, 2, 3, 4, 5, 6, 7, 8, 9, or 10 parts of the first particle to about 1 part of the second particle. The suspension can comprise about 15 parts of the first particle to about 6 parts of the second particle. The parts can be by weight, by volume, or by surface area. A first size of the first particle can be at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of the second particle. A first size of the first particle can be at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of the second particle. The first size can be a first diameter, and the second size can be a second diameter. The first size can be a first average size, and the second size can be a second average size. The first average size and the second average size can be mean sizes or median sizes. A ratio of a mean diameter for the first particle to a mean diameter of the second particle can be about 3:2 to about 2:3.

The first particle, the second particle, or both can be a nanoparticle. The first particle, the second particle, or both can be a microparticle. The first particle, the second particle, or both can be porous. The plurality of biomolecules can comprise at most about 1,000, 100, 10, 1, 0.1, 0.01, or 0.001 nanograms of biomolecules. The plurality of biomolecules can comprise at least about 1,000, 100, 10, 1, 0.1, 0.01, or 0.001 nanograms of biomolecules. The plurality of biomolecules can comprise at most about 1,000, 100, 10, 1, 0.1, 0.01, or 0.001 nanograms of biomolecules per mL. The plurality of biomolecules can comprise at least about 1,000, 100, 10, 1, 0.1, 0.01, or 0.001 nanograms of biomolecules per mL.

In some cases, the compositions, methods, and systems disclosed herein can provide spatially or temporally differential biomolecule compositions of small-volume samples (e.g., individual cells). Spatially differential biomolecule compositions can be obtained by sampling biomolecules from different portions in a cell (e.g., different compartments in a cell) or a tissue (e.g., healthy versus cancerous cells in a tumor, or cells from the epidermis, dermis, and hypodermis of skin). Temporally differential biomolecule compositions can be obtained by sampling biomolecules at different times (e.g., a cell before and after treatment with a potential therapeutic). In some cases, the systems and methods disclosed herein can provide differential biomolecule compositions across a population of subjects (e.g., tumor cells from those treated with a potential chemotherapeutic versus those who have not been treated). Various biomolecules can be targeted (e.g., proteins or nucleic acids) to provide differential transcriptomic or proteomic information between samples. The plurality of biomolecules can comprise biomolecules from at most about 1,000, 100, 10, or 1 cell. The plurality of biomolecules can comprise biomolecules from at least about 1,000, 100, 10, or 1 cell.

The first and the second fluid composition can comprise the same, or different, intensive properties. An intensive property can refer to a property that does not depend on the amount of substance which is measured. An intensive property can be temperature, pressure, density, concentration of constituents, pH, ionic strength, heat capacity, viscosity, surface tension, to give a few examples. In some embodiments, the first fluid composition comprises a first set of intensive physical properties that mediates selective enrichment of the first plurality of biomolecule types. In some embodiments, the second fluid composition comprises a second set of intensive physical properties that mediates selective enrichment of the second plurality of biomolecule types. In some embodiments, the first set of intensive physical properties and the second set of intensive physical properties are different. In some embodiments, the first set of intensive physical properties and the second set of intensive physical properties are the same.

In some embodiments, the first fluid composition comprises a first ratio between a first sample volume and a first surface area of the first surface. In some embodiments, the second fluid composition comprises a second ratio between a second sample volume and a second surface area of the second surface. In some embodiments, the first ratio and the second ratio are different. In some embodiments, the first ratio and the second ratio are the same. A ratio can be, for example, at least 0.05, 0.1, 0.2, 0.3, 0.4, or 0.5 cm² in surface area of a surface per μL of a sample volume. A ratio can be, for example, at most 0.05, 0.1, 0.2, 0.3, 0.4, or 0.5 cm² in surface area of a surface per μL of a sample volume.

In some embodiments, the first fluid composition and the second fluid composition comprise different temperatures. In some embodiments, the first fluid composition and the second fluid composition comprise the same temperature. A temperature can be at least 0, 10, 20, 30, 40, 50, 60, 70, 80, 90, 100, 110, 120, 130, 140, 150, or 160 degrees Celsius. A temperature can be at most 0, 10, 20, 30, 40, 50, 60, 70, 80, 90, 100, 110, 120, 130, 140, 150, or 160 degrees Celsius.

In some embodiments, the first fluid composition comprises a first ionic strength and the second fluid composition comprises a second ionic strength. In some embodiments, the first ionic strength and the second ionic strength are different. In some embodiments, the first ionic strength and the second ionic strength are the same. In some embodiments, the first fluid composition comprises a first ionic concentration and the second fluid composition comprises a second ionic concentration. In some embodiments, the first ionic concentration and the second ionic concentration are different. In some embodiments, the first ionic concentration and the second ionic concentration are the same. An ionic concentration can be at least 0.1, 0.2, 0.3, 0.4, 0.5, 0.6, 0.7, 0.8, 0.9, 1, 2, 3, 4, 5, 6, or 7 M. An ionic concentration can be at most 0.1, 0.2, 0.3, 0.4, 0.5, 0.6, 0.7, 0.8, 0.9, 1, 2, 3, 4, 5, 6, or 7 M.

In some embodiments, the first fluid composition and the second fluid composition comprises different solvents. In some embodiments, the first fluid composition and the second fluid composition comprises the same solvent. A solvent can comprise, e.g., acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol dimethyl ether), 1,2-dimethoxy-ethane (glyme, DME), dimethyl-formamide (DMF), dimethyl sulfoxide (DMSO), 1,4-dioxane, ethanol, ethyl acetate, ethylene glycol, glycerin, heptane, hexamethylphosphoramide, hexamethylphosphorous triamide, hexane, methanol, methyl t-butyl ether, methylene chloride, N-methyl2-pyrrolidinone, nitromethane, pentane, petroleum ether, 1-propanol, 2-propanol, pyridine, tetrahydrofuran, toluene, triethyl amine, water, o-xylene, m-xylene, p-xylene, or any combination thereof. In some embodiments, the first fluid composition, the second fluid composition, or both comprise a buffer comprising tris(hydroxymethyl) aminomethane. In some embodiments, the first fluid composition, the second fluid composition, or both comprise a buffer comprising citrate. In some embodiments, the first fluid composition, the second fluid composition, or both comprise a buffer comprising glycine. In some embodiments, the first fluid composition, the second fluid composition, or both comprise a buffer comprising phosphate. In some embodiments, the first fluid composition, the second fluid composition, or both comprise a buffer comprising carbonate. In some embodiments, the first fluid composition, the second fluid composition, or both comprise a buffer comprising N-cyclohexyl-3-aminopropanesulfonic acid. In some embodiments, the first fluid composition, the second fluid composition, or both comprise a buffer comprising 3-((3-cholamidopropyl) dimethylammonio)-1-propanesulfonate. In some embodiments, the first fluid composition comprises an aqueous buffer. In some embodiments, the second fluid composition comprises an aqueous buffer. In some embodiments, the first fluid composition comprises an aqueous buffer and the second fluid composition does not comprise a buffer.

In some embodiments, the first fluid composition and the second fluid composition comprise different salts. In some embodiments, the first fluid composition and the second fluid composition comprise the same salt. A salt can comprise, e.g., ammonium sulfate, sodium sulfate, sodium chloride, potassium chloride, ammonium acetate, etc.

In some embodiments, the first fluid composition comprises a first pH, and the second fluid composition comprises a second pH. In some embodiments, the first pH and the second pH are the same. In some embodiments, the first pH and the second pH are different. In some embodiments, the first fluid composition, the second fluid composition, or both comprise a pH between 2 and 4. In some embodiments, the first fluid composition, the second fluid composition, or both comprise a pH between 5 and 7. In some embodiments, the first fluid composition, the second fluid composition, or both comprise a pH between 9 and 10. In some embodiments, the first fluid composition, the second fluid composition, or both comprise a pH of at least about 1, 2, 3, 4, 5, 6, 7, 8, 9, 10, 11, 12, 13, or 14. In some embodiments, the first fluid composition, the second fluid composition, or both comprise a pH of at most about 1, 2, 3, 4, 5, 6, 7, 8, 9, 10, 11, 12, 13, or 14. In some embodiments, a method can further comprise diluting a sample to make the first composition, the second composition, or both. In some embodiments, the diluting comprises adding a buffer. In some embodiments, the buffer comprises a pH of at least about 1, 2, 3, 4, 5, 6, 7, 8, 9, 10, 11, 12, 13, or 14. In some embodiments, the buffer comprises a pH of at most about 1, 2, 3, 4, 5, 6, 7, 8, 9, 10, 11, 12, 13, or 14. In some embodiments, the sample and the buffer comprises different pH. In some embodiments, the sample and the buffer comprises about the same pH. In some embodiments, the second composition has a pH between 5 and 9. In some embodiments, the second composition has a pH between 6 and 8. In some embodiments, the second composition has a pH between 8 and 11. In some embodiments, the second composition has a pH between 9 and 10. In some embodiments, the second composition does not comprise a buffer. In some embodiments, the second composition comprises a buffer that is different from the buffer configured to maintain a pH of the composition between 8 and 11. In some embodiments, the second composition comprises the same buffer as the composition. In some embodiments, the composition and the second composition both comprise Tris buffer having a pH between 9 and 10. In some embodiments, the incubating of the composition and the incubating of the second composition occur at the same time. In some embodiments, the composition comprises a buffer. In some embodiments, the buffer is configured to maintain a pH of the composition between 8 and 11. In some embodiments, the buffer is configured to maintain a pH of the composition between 9 and 10. In some embodiments, the buffer is configured to maintain a pH of the composition of about 9.5. In some embodiments, the buffer is Tris buffer. In some embodiments, the buffer is an aqueous buffer.

The compositions and/or properties of the first fluid composition, the second fluid composition, or both can be used as features for identifying a biological state associated with a sample. For instance, a feature can be used in a machine learning algorithm. The feature can comprise, the identity of a biomolecule, the particle(s) used to assay the biomolecule, the temperature, pH, and the like. Thus, the various compositions and/or properties can provide additional features to stratify biological samples for distinguishing biological states. In some embodiments, the method further comprises identifying a biological state associated with the first fluid composition based at least partially on one or more physical properties of the first fluid composition. In some embodiments, the method further comprises identifying a biological state associated with the second fluid composition based at least partially on one or more physical properties of the second fluid composition. In some embodiments, the method further comprises identifying a biological state associated with the first fluid composition based at least partially on one or more physical properties of the first wash composition. In some embodiments, the method further comprises identifying a biological state associated with the second fluid composition based at least partially on one or more physical properties of the second wash composition. In some embodiments, the method further comprises identifying a biological state associated with the first fluid composition based at least partially on one or more physical properties of the first reconstitution composition. In some embodiments, the method further comprises identifying a biological state associated with the second composition based at least partially on one or more physical properties of the second reconstitution composition.

In some aspects, the present disclosure provides a kit for selectively enriching and assaying a plurality of biomolecules in a fluid composition. In some embodiments, the kit comprises a first reagent comprising a first pH. In some embodiments, the kit comprises a second reagent comprising a second pH. In some embodiments, the kit comprises a first surface configured to selectively enrich a first plurality of biomolecule types in a first fluid composition comprising the first surface and the first reagent. In some embodiments, the kit comprises a second surface configured to selectively enrich a second plurality of biomolecule types in a second fluid composition comprising the second surface and the second reagent. In some embodiments, the first fluid composition comprises the first reagent. In some embodiments, the second fluid composition comprises the second reagent. In some embodiments, the first pH and the second pH are different.

In some aspects, the present disclosure provides a kit comprising a first composition comprising a first particle and second particle. The first particle can have a first functionalized surface chemistry that is different from a second functionalized surface chemistry of the second particle. Both the first particle and the second particle can have a negative charge. The first composition can be a solid. The kit can further comprise a second composition comprising a third particle and fourth particle. The third particle can have a third functionalized surface chemistry that is different from a fourth functionalized surface chemistry of the fourth particle. Both the third particle and the fourth particle can have a positive charge. The third composition can be a solid.

The kit can comprise a buffer having a pH between 9 and 10. The kit can comprise a Tris buffer having a pH between 9 and 10. Both the first composition and the second composition can be lyophilized. In some embodiments, the first particle is a magnetic particle and comprises an outer layer with a silanol surface functionalization. In some embodiments, the second particle is a magnetic particle and comprises an outer layer with a carboxylate surface functionalization. In some embodiments, third particle is a magnetic particle and comprises an outer layer with an amine surface functionalization. In some embodiments, the fourth particle is a magnetic particle and comprises an outer layer with an amine surface functionalization. In some embodiments, the outer layer of the third particle comprises a polymer. In some embodiments, the first composition has a ratio of about 10 to about 15 parts by weight of the first particle to about 1 part by weight of the second particle. In some embodiments, the second composition has a ratio of about 10 to about 15 parts by weight of the third particle to about 1 part by weight of the fourth particle. In some embodiments, a ratio of a mean diameter for the first particle to a mean diameter of the second particle is about 3:2 to about 2:3. In some embodiments, a ratio of a mean diameter for the third particle to a mean diameter of the fourth particle is about 3:2 to about 2:3. In some embodiments, each of the first particle, the second particle, the third particle, and the fourth particle are superparamagnetic iron oxide nanoparticles. In some embodiments, the first composition has a ratio of about 10 to about 15 parts by weight of the first particle to about 1 part by weight of the second particle. In some embodiments, a ratio of a mean diameter for the first particle to a mean diameter of the second particle is about 3:2 to about 2:3. In some embodiments, the first particle and the second particle are superparamagnetic iron oxide nanoparticles.

In some aspects, the present disclosure provides a composition for selectively enriching and assaying a plurality of biomolecules in a fluid composition. In some embodiments, the composition comprises a suspension. In some embodiments, the suspension comprises a first particle. In some embodiments, the first particle comprises a first paramagnetic portion. In some embodiments, the first particles comprises a first surface chemistry. In some embodiments, the suspension comprises a second particle. In some embodiments, the second particle comprises a second paramagnetic portion. In some embodiments, the second particle comprises second surface chemistry. In some embodiments, the second surface chemistry and the first surface chemistry are different. In some embodiments, the suspension comprises a plurality of biomolecules adsorbed on the first particle. In some embodiments, the suspension comprises a plurality of biomolecules adsorbed on the second particle.

In some aspects, the present disclosure provides a suspension. In some embodiments, the suspension comprise first particles. In some embodiments, the first particles comprise an outer layer with a negatively charged surface functionalization. In some embodiments, the first particles comprise a first outer layer with a positively charged surface functionalization. In some embodiments, the suspension comprises second particles comprising a second outer layer with a negatively charged surface functionalization. In some embodiments, the second particles comprise a second outer layer with a positively charged surface functionalization. In some embodiments, both the first particle and the second particle have a negative charge. In some embodiments, both the first particle and the second particle have a positive charge. In some embodiments, the biomolecules are adsorbed to the outer layer of the first particles. In some embodiments, the second outer layer is different from the first outer layer. In some embodiments, the biomolecules are adsorbed to the second outer layer of the second particles. In some embodiments, the biomolecules are adsorbed to the first outer layer of the first particles and the second outer layer of the particles. In some embodiments, the first particles comprises a first functionalized surface chemistry. In some embodiments, the second particles comprise a second functionalized surface chemistry. In some embodiments, the first functionalized surface chemistry is different from the second functionalized surface chemistry of the second particle. In some embodiments, first outer layer comprises silanol. In some embodiments, the second outer layer comprises a carboxylate. In some embodiments, the first and second outer layer comprise an amine group. In some embodiments, the first particle is a magnetic particle and comprises an outer layer with an amine surface functionalization. In some embodiments, the second particle is a magnetic particle and comprises an outer layer with an amine surface functionalization. In some embodiments, the second outer layer is different from the first outer layer. In some embodiments, the biomolecules comprise at least 100 different peptides or proteins. In some embodiments, the first particles and optionally the second particles are magnetic.

In some embodiments, the suspension comprises a buffer. In some embodiments, the buffer is configured to maintain a pH of the suspension between 9 and 10. In some embodiments, the buffer is Tris buffer. In some embodiments, the buffer is configured to maintain a pH of about 8 to about 11. In some embodiments, the buffer is configured to maintain a pH between 9 and 10. In some embodiments, the buffer is Tris buffer having a pH between 9 and 10. In some embodiments, the first particle is a magnetic particle and comprises an outer layer with a silanol surface functionalization, and the second particle is a magnetic particle and comprises an outer layer with a carboxylate surface functionalization.

In some embodiments, the outer layer of the first particle comprises a polymer. In some embodiments, the suspension has a ratio of about 10 to about 15 parts by weight of the first particle to about 1 part by weight of the second particle. In some embodiments, a ratio of a mean diameter for the first particle to a mean diameter of the second particle is about 3:2 to about 2:3. In some embodiments, the first particle and the second particle are superparamagnetic iron oxide nanoparticles.

In some aspects, the present disclosure provides a system for analyzing a plurality of biological samples. In some embodiments, the system comprises a plurality of partitions comprising a first partition and a second partition. In some embodiments, the system comprises a plurality of reagent storages comprising a first reagent and a second reagent. In some embodiments, the first reagent comprises a first pH. In some embodiments, the second reagent comprises a second pH. In some embodiments, the first pH and the second pH are different. In some embodiments, the system comprises a plurality of substrates comprising a first substrate and a second substrate. In some embodiments, the first substrate comprises a first surface chemistry. In some embodiments, the second substrate comprises a second surface chemistry. In some embodiments, the system comprises one or more transfer devices operably connected to the plurality of partitions, the plurality of reagent storages, the plurality of substrates, or any combination thereof. In some embodiments, the system comprises a computer comprising at least one processor and instructions executable by the at least one processor to perform steps for analyzing the plurality of biological samples. In some embodiments, the instructions may comprise generating, using the one or more transfer devices, a first fluid composition in the first partition comprising the first substrate, the first reagent, and a first plurality of biomolecules. In some embodiments, the first plurality of biomolecules is adsorbed on the first substrate. In some embodiments, the instructions may comprise generating, using the one or more transfer devices, a second fluid composition in the second partition comprising the second substrate, the second reagent, and a second plurality of biomolecules. In some embodiments, the second plurality of biomolecules is adsorbed on the second substrate. In some embodiments, the instructions may comprise preparing, using the one or more transfer devices, the first plurality of biomolecules and the second plurality of biomolecules for mass spectrometry.

In some aspects, the present disclosure provides a system for analyzing a plurality of biological samples. In some embodiments, the system comprises a partition. In some embodiments, the system comprises a reagent storage comprising a reagent; In some embodiments, the system comprises a plurality of substrates comprising a first substrate comprising a first surface chemistry and a second substrate comprising a second surface chemistry. In some embodiments, the first surface chemistry and the second surface chemistry are different. In some embodiments, the system comprises one or more transfer devices operably connected to the partition, the reagent storage, the plurality of substrates, or any combination thereof. In some embodiments, the system comprises a computer comprising at least one processor and instructions executable by the at least one processor to perform steps for analyzing the plurality of biological samples. In some embodiments, the instructions may comprise generating, using the one or more transfer devices, a fluid composition in the partition comprising the plurality of substrates, the reagent, and a plurality of biomolecules, wherein the plurality of biomolecules is adsorbed on the substrate. In some embodiments, the instructions may comprise preparing, using the one or more transfer devices, the plurality of biomolecules for mass spectrometry.

### Biological Sample

The sample can be various in amount. In some embodiments, the sample comprises at most about 1,000, 100, 10, 1, 0.1, 0.01, or 0.001 nanograms of biomolecules. In some embodiments, the sample comprises at least about 1,000, 100, 10, 1, 0.1, 0.01, or 0.001 nanograms of biomolecules. In some embodiments, the sample comprises at most about 1,000, 100, 10, 1, 0.1, 0.01, or 0.001 nanograms of biomolecules per mL of the sample. In some embodiments, the sample comprises at least about 1,000, 100, 10, 1, 0.1, 0.01, or 0.001 nanograms of biomolecules per mL of the sample. In some embodiments, the sample comprises biomolecules from at most about 1,000, 100, 10, or 1 cell. In some embodiments, the sample comprises biomolecules from at least about 1,000, 100, 10, or 1 cell. In some embodiments, the sample comprises at most about 1,000, 100, 10, 1, 0.1, 0.01, or 0.001 microliters. In some embodiments, the sample comprises at least about 1,000, 100, 10, 1, 0.1, 0.01, or 0.001 microliters.

The compositions and/or the properties of the first and the second fluid compositions can be designed to increase the dynamic range and/or the number of peptides, proteins, or protein groups detected using the PROTEOGRAPH™ for different types of samples. In some embodiments, the sample comprises a complex biological sample. The complex biological sample can comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample comprises plasma.

In some embodiments, the first plurality of biomolecule types, the second plurality of biomolecule types, or both comprise one or more polyamino acids. A polyamino acid can refer to any biomolecule comprising two or more amino acids. The amino acids can be the same or different. The amino acids can be covalently bonded. The covalent bond can be a peptide bond. A polyamino acid can comprise a peptide, a protein, or both.

In some embodiments, the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some embodiments, the biological sample is plasma or serum. In some embodiments, the biological sample is cell culture media. In some embodiments, the composition comprises no more than 60% by volume of the biological sample. In some embodiments, the composition comprises no more than 25% by volume of the biological sample. In some embodiments, the composition comprises no more than 15% by volume of the biological sample. In some embodiments, the composition comprises at least 1% by volume of the biological sample. In some embodiments, the composition comprises at least 4% by volume of the biological sample. In some embodiments, the composition comprises at least 10% by volume of the biological sample. In some embodiments, the composition comprises at least 20% by volume of the biological sample.

The present disclosure systems and methods for assaying a biological sample. In some cases, a biological sample may comprise a cell or be cell-free. In some cases, a biological sample may comprise a biofluid, such as blood, serum, plasma, urine, or cerebrospinal fluid (CSF). In some cases, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract. In some cases, a biological sample may be obtained from a subject. In some cases, the subject may be a human or a non-human. In some cases, the subject may be a plant, a fungus, or an archaeon. In some cases, a biological sample can contain a plurality of proteins or proteomic data, which may be analyzed after adsorption or binding of proteins to the surfaces of the various sensor element (e.g., particle) types in a panel and subsequent digestion of protein coronas.

In some cases, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, a biological sample may comprise multiple biological samples (e.g., pooled plasma from multiple subjects, or multiple tissue samples from a single subject). In some cases, a biological sample may comprise a single type of biofluid or biomaterial from a single source.

In some cases, a biological sample may be diluted or pre-treated. In some cases, a biological sample may undergo depletion (e.g., the biological sample comprises serum) prior to or following contact with a surface disclosed herein. In some cases, a biological sample may undergo physical (e.g., homogenization or sonication) or chemical treatment prior to or following contact with a surface disclosed herein. In some cases, a biological sample may be diluted prior to or following contact with a surface disclosed herein. In some cases, a dilution medium may comprise buffer or salts, or be purified water (e.g., distilled water). In some cases, a biological sample may be provided in a plurality of partitions, wherein each partition may undergo different degrees of dilution. In some cases, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1,000-fold dilution. In some cases, the biological sample may be diluted using a buffer that modified pH of the biological sample. In some case, the pH may be modified to about 2, 3, 4, 5, 6, 7, 8, 9, 10. In some case, the pH may be modified to 9-10. In some cases, the pH is modified by dilution with a pH 9.5 Tris buffer. In some cases, the biological sample be separated into portions, and the portions may be adjusted to different pHs before processing using the methods disclosed herein.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

### Proteomic Analysis

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a particle coated with an outer layer of silica and a particle coated with an outer layer of poly(dimethyl aminopropyl methacrylamide) (PDMAPMA). A particle panel consistent with the present disclosure could also comprise two or more particles selected from the group consisting of silica coated particle, an N-(3-Trimethoxysilylpropyl) diethylenetriamine coated particle, a PDMAPMA coated particle, a carboxyl-functionalized polyacrylic acid coated particle, an amino surface functionalized particle, a polystyrene carboxyl functionalized particle, and a dextran coated particle. A particle panel consistent with the present disclosure may also comprise two or more particles selected from the group consisting of a surfactant free carboxylate functionalized particle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl) ammonium hydroxide, P(DMAPMA-co-SBMA) coated particle. A particle panel consistent with the present disclosure may comprise silica-coated particles, N-(3-Trimethoxysilylpropyl) diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof. In some cases, the particle panel may comprise silica-coated particles, amine-functionalized particles, amine-functionalized polymer-coated particles, and carboxylate-functionalized particles.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl) diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group, a carbamoyl group, a hydroxyl group, an aldehyde group and the like. In some cases, a polar functional group may comprise a primary amine group, a secondary amine group, a tertiary amine group, a quaternary amine group, a cyclic secondary amine group, a primary amide group, a secondary amide group, a tertiary amide group, an imine group, a pyridyl group, a pyrimidine group, a pyrrolidinium group, an imidazole group, a guanidine group, a guanidinium group, or any combination thereof.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle. In some cases, a positively charged particle may have a zeta potential of more than 0, 5, 10, 15, 20, 25, 50, or 100 mV. In some cases, a negative charged particle may have a zeta potential of less than 0, −5, −10, −15, −20, −25, −50, or −100 mV.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona A protein corona analysis may comprise identifying one or more proteins in the biomolecule corona, for example by mass spectrometry. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1,000, 2,000, 3,000, 4,000, 5,000, 6,000, 7,000, 8,000, 9,000, or 10,000 unique biomolecules. In some cases, a surface disclosed herein may be incubated with a biological sample to adsorb at most 1,000, 2,000, 3,000, 4,000, 5,000, 6,000, 7,000, 8,000, 9,000, or 10,000 unique biomolecules. In some cases, a surface disclosed herein may be incubated with a biological sample to adsorb at least 1,000, 2,000, 3,000, 4,000, 5,000, 6,000, 7,000, 8,000, 9,000, or 10,000 unique biomolecule groups. In some cases, a surface disclosed herein may be incubated with a biological sample to adsorb at most 1,000, 2,000, 3,000, 4,000, 5,000, 6,000, 7,000, 8,000, 9,000, or 10,000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, or 10,000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, or 10,000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, or 10,000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, or 10,000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry).

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 unique proteins. In some cases, the panels disclosed herein can be used to identify at most 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 unique proteins. In some cases, the panels disclosed herein can be used to identify at least 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 protein groups. In some cases, the panels disclosed herein can be used to identify at most 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 protein groups. In some cases, the panels disclosed herein can be used to identify at least 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, or 1,000,000 peptides. In some cases, the panels disclosed herein can be used to identify at most 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, or 1,000,000 peptides. In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papraine, Asp N, thermolysin, pepsin, aspartyl protease, cathepsin D, zinc metalloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, proteins or peptides may be prepared for mass spectrometry. In some cases, proteins or peptides may be treated with an alkylating agent. For example, proteins or peptides may be treated with N-ethylmaleimide and iodoacetamide. In some case, proteins or peptides may be treated with a reducing agent. For example, proteins or peptides are treated with dithiothreitol (DTT) or tris(2-carboxyethyl) phosphine (TCEP). In some cases, proteins or peptides are digested, alkylated, and reduced before analysis. In some cases, proteins or peptides are digested, alkylated, and reduced before analysis using mass spectrometry.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl) diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3,000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1,000 cm²/mg, 500 to 2,000 cm²/mg, 1,000 to 4,000 cm²/mg, 2,000 to 8,000 cm²/mg, or 4,000 to 10,000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some cases, a particle may comprise a nanoparticle. In some cases, a particle may comprise a microparticle. In some cases, a first particle, a second particle, or both particles in a particle panel are nanoparticles. In some cases, a first particle, a second particle, or both particles in a particle panel are microparticles. In some cases, a first particle may be a nanoparticle and a second particle may be a microparticle.

In some cases, a particle may comprise a diameter of at least about 10, 20, 30, 40, 50, 60, 70, 80, 90, 100, 200, 300, 400, 500, 600, 700, 800, 900, or 1,000 nm. In some cases, a particle may comprise a diameter of at most about 10, 20, 30, 40, 50, 60, 70, 80, 90, 100, 200, 300, 400, 500, 600, 700, 800, 900, or 1,000 nm. In some cases, a particle may comprise a diameter of at least about 10, 20, 30, 40, 50, 60, 70, 80, 90, 100, 200, 300, 400, 500, 600, 700, 800, 900, or 1,000 μm. In some cases, a particle may comprise a diameter of at most about 10, 20, 30, 40, 50, 60, 70, 80, 90, 100, 200, 300, 400, 500, 600, 700, 800, 900, or 1,000 μm.

In some cases, a first size of a first particle is at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of a second particle. In some cases, a first size of a first particle is at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of a second particle. In some cases, a size of a first particle is within ±40% of a size of a second particle, a size of a first particle is within ±30% of a size of a second particle, a size of a first particle is within ±25% of a size of a second particle, a size of a first particle is within ±20% of a size of a second particle, a size of a first particle is within ±15% of a size of a second particle, or a size of a first particle is within ±10% of a size of a second particle. In some cases, the first size is a first diameter, and the second size is a second diameter. In some cases, the first size is a first average size, and the second size is a second average size. In some cases, the first average size and the second average size are mean sizes or median sizes.

In some cases, a particle panel comprises at least about 1, 2, 3, 4, 5, 6, 7, 8, 9, 10, or 11 parts of a first particle to about 1 part of a second particle. In some cases, a particle panel comprises at most about 1, 2, 3, 4, 5, 6, 7, 8, 9, 10, 11, 12, 13, 14, 15, or 20 parts of a first particle to about 1 part of a second particle. In some cases, a particle panel comprises about 15 parts of a first particle to about 6 parts of a second particle. In some cases, the parts are parts by weight, parts by volume, or parts by surface area. In some cases, the parts are parts by weight. In some cases, a particle panel comprises about 5 to about 20 parts by weight of a first particle to about 1 part by weigh of a second particle. In some cases, a particle panel comprises about 10 to about 15 parts by weight of a first particle to about 1 part by weigh of a second particle.

### Proteomic Information

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof.

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and introns in the code of life. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof.

In some cases, splicing variant (in some cases also referred to as “alternative splicing” variants, “differential splicing” variants, or “alternative RNA splicing” variants) may refer to a protein that is expressed by an alternative splicing process. In some cases, an alternative splicing process may express one or more splicing variants from a set of exons via different combinations of exons. In some cases, a combination may comprise a different sequence of exons compared to another combination. In some cases, a combination may comprise a different subset of exons compared to another combination. In some cases, a splicing variant may comprise a reordered amino acid sequence of another splicing variant.

In some cases, an allelic variant may refer to a protein that is expressed from a gene comprising a mutation compared to a reference gene. In some cases, the reference gene may be the gene of a cell, an individual, or a population of individuals. In some cases, the mutation may be a base substitution, a base deletion, or a base insertion of a genetic sequence of the gene compared to a genetic reference of the reference gene. In some cases, an allelic variant may comprise an amino acid substitution in an amino acid sequence of another allelic variant.

In some cases, a post-translation modification may refer to a protein that is modified after expression. A protein may be modified by various enzymes. In some cases, an enzyme that can modify a protein may be a kinase, a protease, a ligase, a phosphatase, a transferase, a phosphotransferase, or any other enzyme for performing the any one of modifications disclosed herein.

In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, glutathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some cases, proteomic information may be encoded as digital information. In some cases, the proteomic information may comprise one or more elements that represents the proteomic information. In some cases, an element may represent a primary structure information, secondary structure information, tertiary structure information, or quaternary information about a peptide or a protein. In some cases, an element may represent protein-ligand interactions for a peptide or a protein. In some cases, an element may represent a source of a peptide or protein (e.g., a specific cell, tissue, organ, organism, individual, or population of individuals). In some cases, an element may represent a type of proteoform. In some cases, an element may be a number, a vector, an array, or any other datatypes provided herein.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1,000 fg/mm2. In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1,000 pg/mm2. In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1,000 ng/mm2. In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1,000 μg/mm2. In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1,000 mg/mm2. In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1,000 fg/mm2. In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1,000 μg/mm2. In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1,000 ng/mm2. In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1,000 μg/mm2. In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1,000 mg/mm2.

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, or 10,000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, or 10,000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

### Composition Improving Assay

In some cases, a method of the present disclosure may comprise using a composition improving assay. In some cases, an untargeted assay may be a composition improving assay. In some cases, a composition improving assay may improve access to a subset of biomolecules in a biological sample. In some cases, a composition improving assay may improve detection to a subset of biomolecules in a biological sample. In some cases, a composition improving assay may improve identification to a subset of biomolecules in a biological sample. In some cases, the subset of biomolecules may be low-abundance biomolecules. In some cases, the subset of biomolecules may be rare biomolecules. In some cases, a dynamic range of a biological sample may be compressed using a composition improving assay. In some cases, a dynamic range may be compressed by at least about 1, 2, 3, 4, 5, 6, 7, 8, 9, 10, 11, 12, 13, 14, or 15 magnitudes.

In some cases, the composition improving assay may comprise providing one or more of surface regions comprising one or more surface types. In some cases, the composition improving assay may comprise contacting the biological sample with the one or more surface regions to yield a set of adsorbed biomolecules on the one or more surface regions. In some cases, the composition improving assay may comprise desorbing, from the one or more surface regions, at least a portion of the set of adsorbed biomolecules to yield the set of polyamino acids. In some cases, the composition improving assay may comprise contacting the biological sample with the one or more surface regions to capture a set of biomolecules on the one or more surface regions. In some cases, the composition improving assay may comprise releasing, from the one or more surface regions, at least a portion of the set of biomolecules to yield the set of polyamino acids. In some cases, the one or more surface regions are disposed on a single continuous surface. In some cases, the one or more surface regions are disposed on one or more discrete surfaces. In some cases, the one or more discrete surfaces are surfaces of one or more particles. In some cases, the one or more particles may comprise a nanoparticle. In some cases, the one or more particles may comprise a microparticle. In some cases, the one or more particles may comprise a porous particle. In some cases, the one or more particles may comprise a bifunctional, trifunctional, or N-functional particle.

In some cases, the composition improving assay may comprise providing a plurality of surface regions comprising a plurality of surface types. In some cases, the composition improving assay may comprise contacting the biological sample with the plurality of surface regions to yield a set of adsorbed biomolecules on the plurality of surface regions. In some cases, the composition improving assay may comprise desorbing, from the plurality of surface regions, at least a portion of the set of adsorbed biomolecules to yield the set of polyamino acids. In some cases, the composition improving assay may comprise contacting the biological sample with the plurality of surface regions to capture a set of biomolecules on the plurality of surface regions. In some cases, the composition improving assay may comprise releasing, from the plurality of surface regions, at least a portion of the set of biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles. In some cases, the plurality of particles may comprise a nanoparticle. In some cases, the plurality of particles may comprise a microparticle. In some cases, the plurality of particles may comprise a porous particle. In some cases, the plurality of particles may comprise a bifunctional, trifunctional, or N-functional particle.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Protein Profiling of Biological Fluids with Proteograph™ Assay Using an Alternative pH Condition for Corona Incubation for Different Nanoparticles

Biological fluid samples can be incubated with different nanoparticles (NPs) in solutions comprising water and/or biological pH to form protein coronas on the NPs. This example provides using alternative fluid compositions with different pH with different NPs to improve binding between NPs and proteins in the biological fluid samples. In some cases, using the alternative fluid compositions may improve depth of proteome coverage.

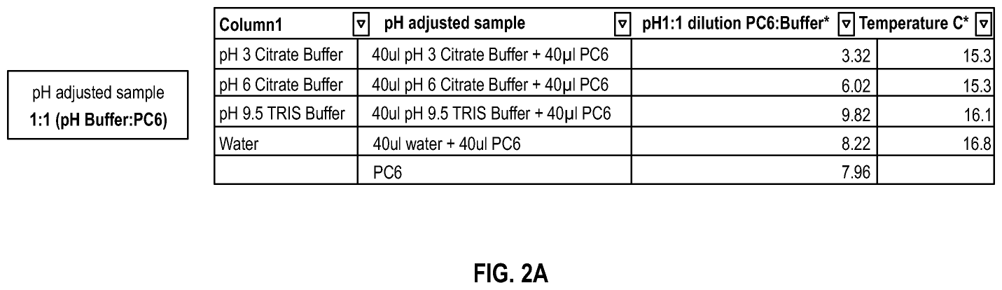

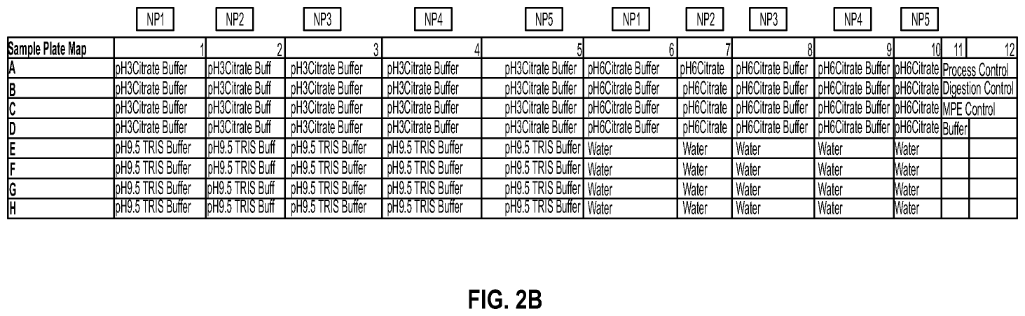

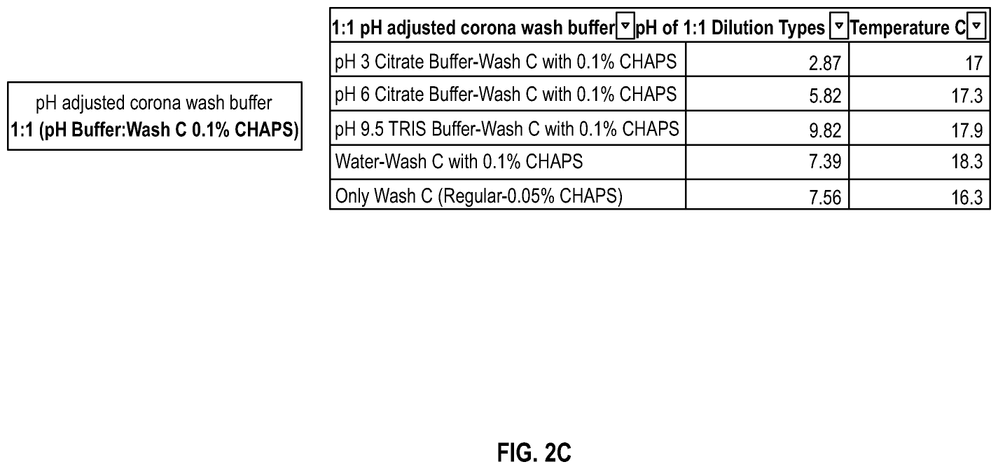

Plasma samples with various pH buffers were mixed at a same volume ratio, as shown in FIG. 2A-2B. The mixtures were added to a dry NP pellet for incubation. After completing the incubation, the supernatant was removed from mixture to isolate the NPs. The coronas of the NPs were washed with pH adjusted wash buffers to wash loosely bound proteins off surfaces of the coronas. The NPs were washed with the wash compositions shown in FIG. 2C. After, digestion and cleanup steps were performed on the proteins of the coronas. The proteins were analyzed using LC-MS/MS using a PharmaFluidics column under data dependent acquisition (DDA) mode, with maximum injection mass per condition. Various environments for corona formation was investigated with different NPs to reach deeper proteome depth.

The data showed that, by using some pH buffers, 30 to 40 percent increase in protein group counts were observed for several NPs, as shown in FIG. 3. About 42% increase in protein group numbers were observed for NP 2 with a corona conditions with pH of 9.5 Tris buffer and 48% increase in protein group numbers for NP 5 at same conditions.

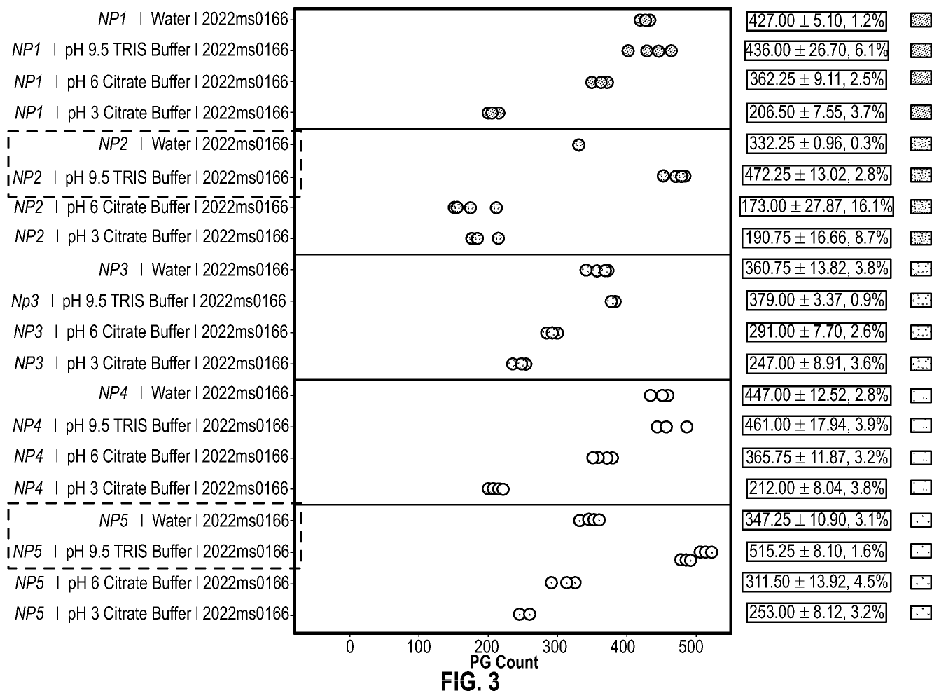

The data showed that, by using some pH buffers, peptide yield was increased, as shown in FIG. 4. Using citrate buffer with pH of 3, the peptide yield was increased for all nanoparticles tested.

The data showed that, by using some pH buffers, miscleavage ratio of was increased, as shown in FIG. 5. Using citrate buffer with pH of 3, the miscleavage ratio was increased for all nanoparticles tested.

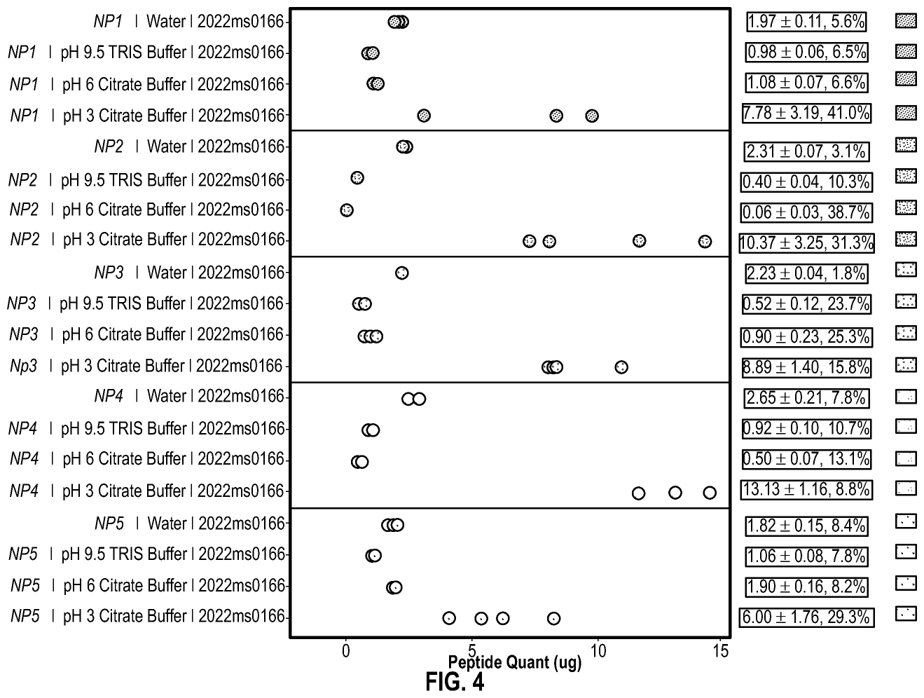

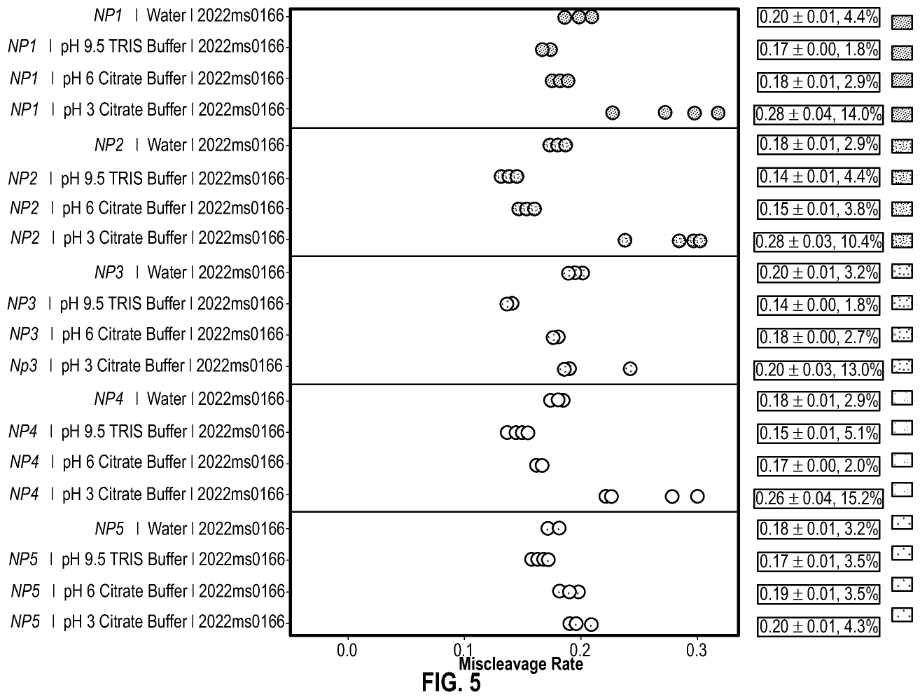

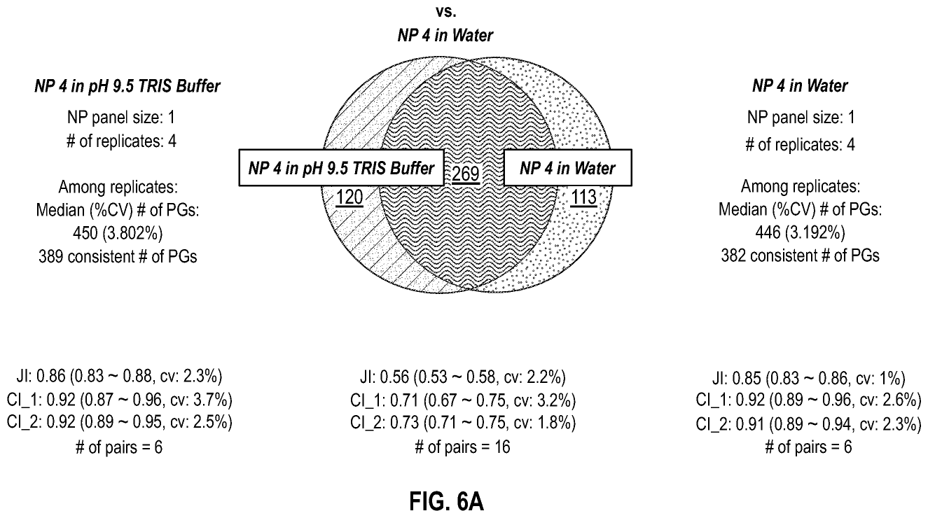

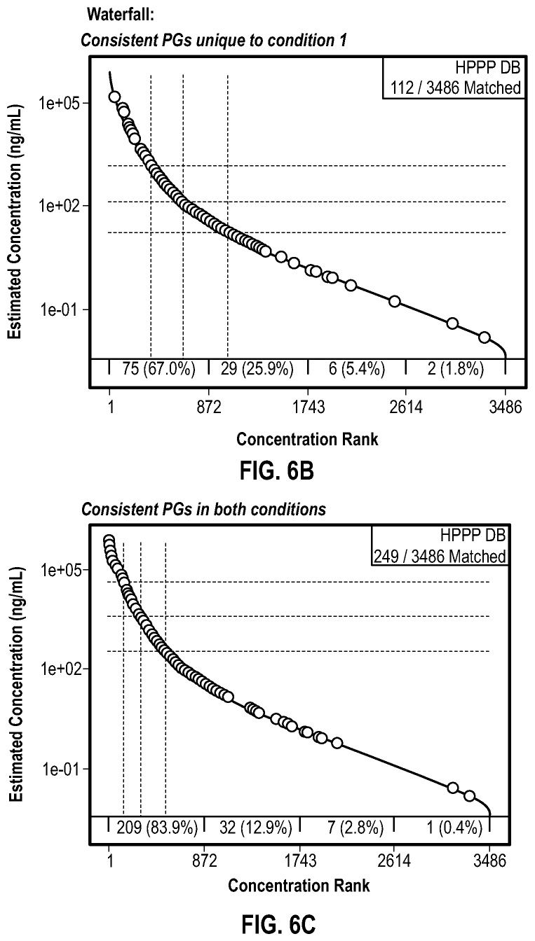

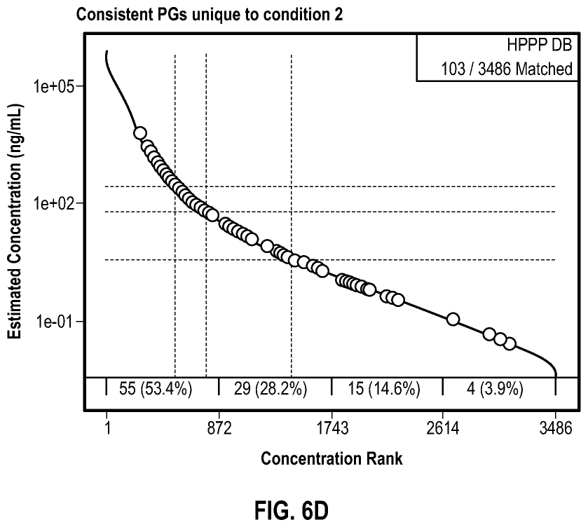

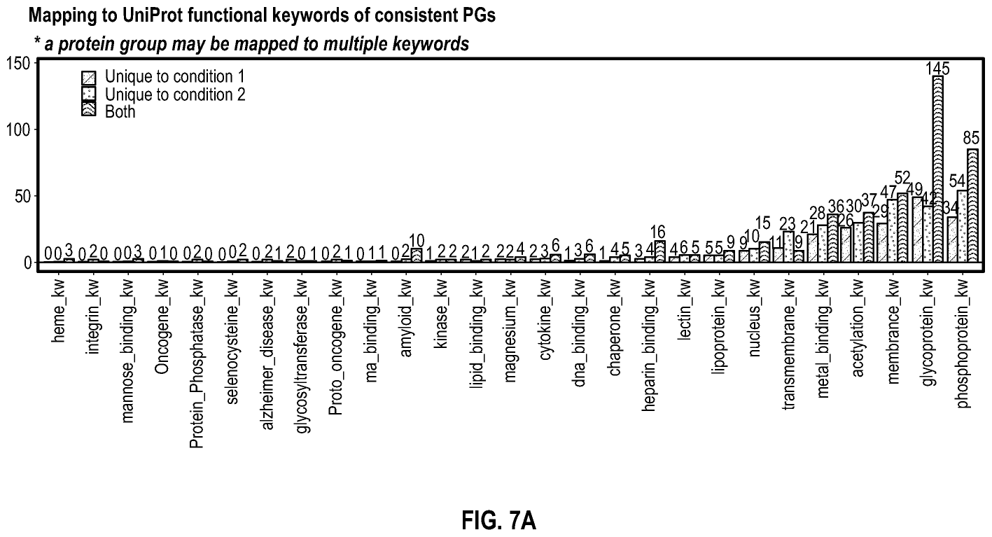

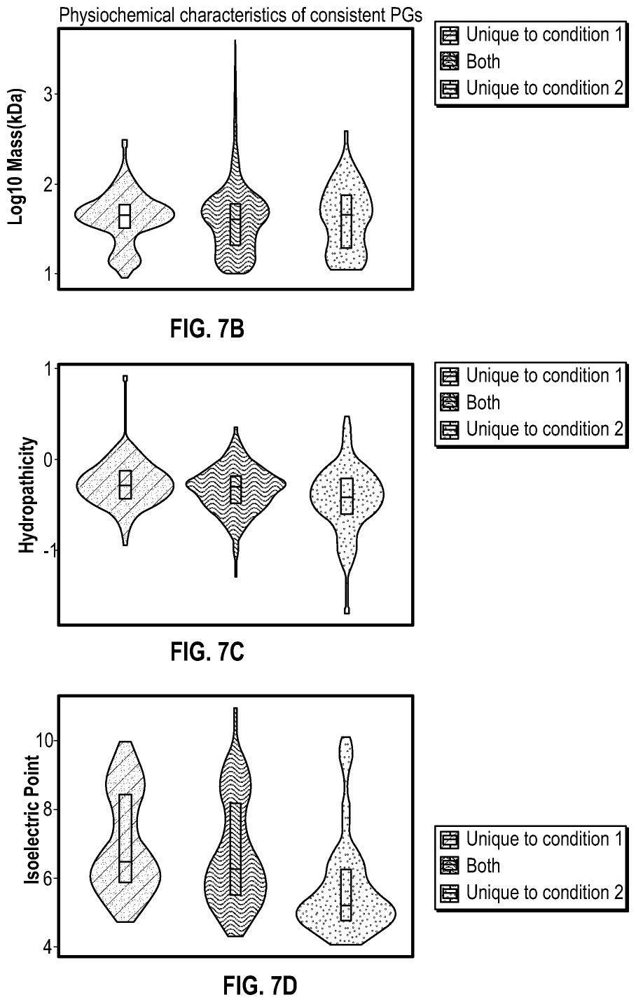

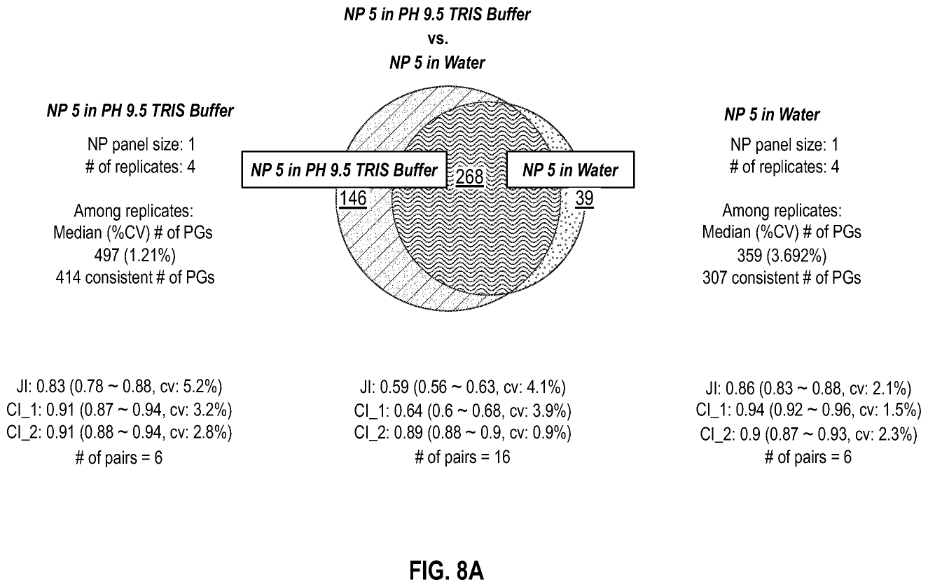

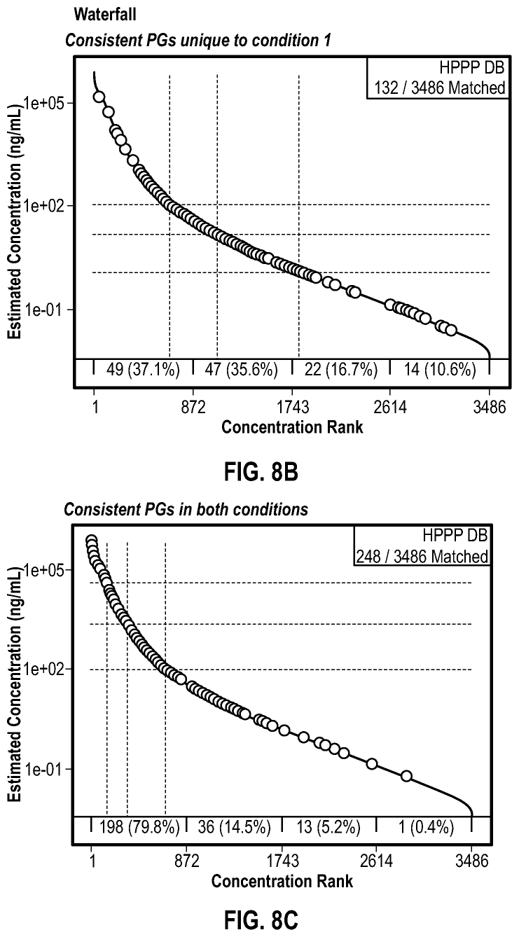

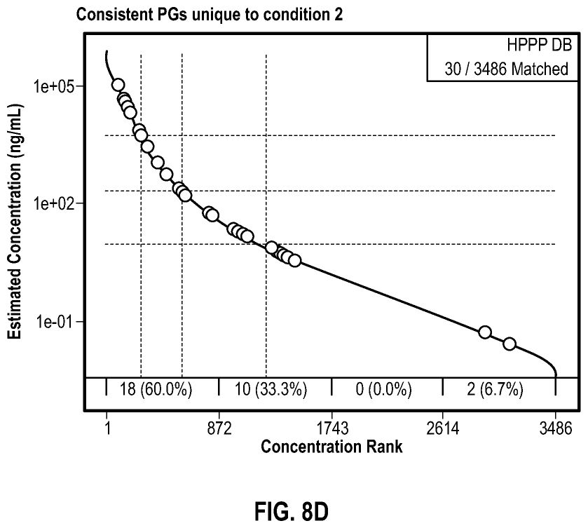

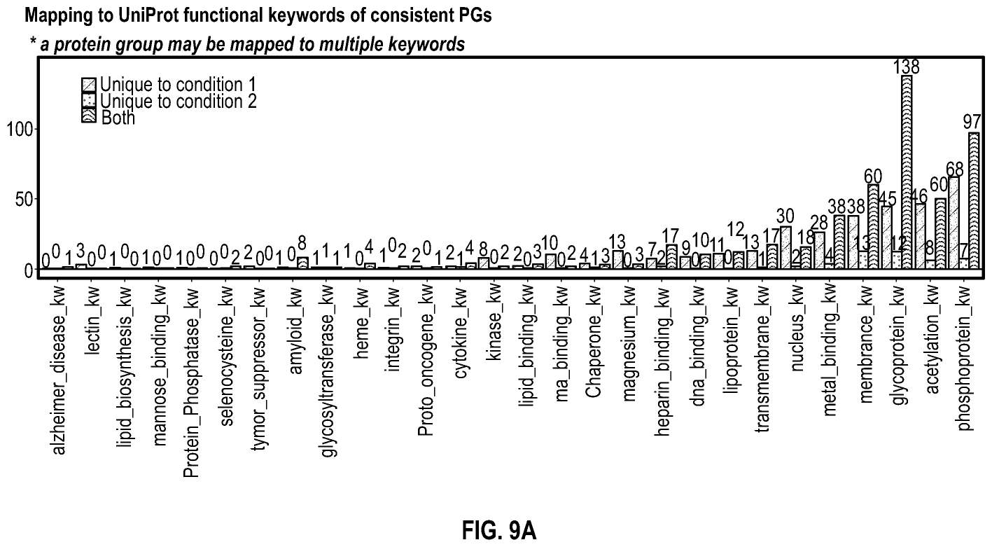

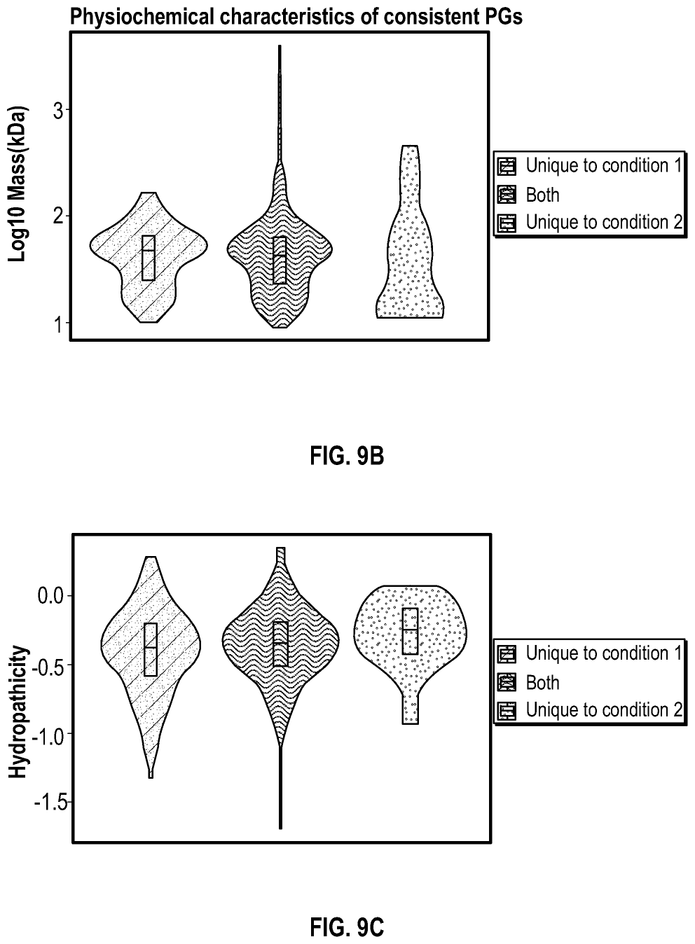

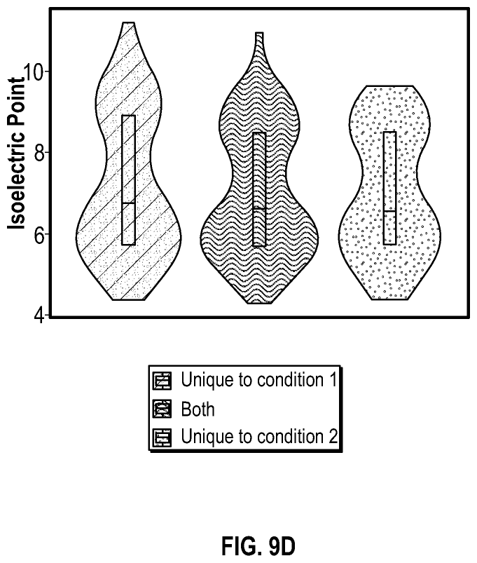

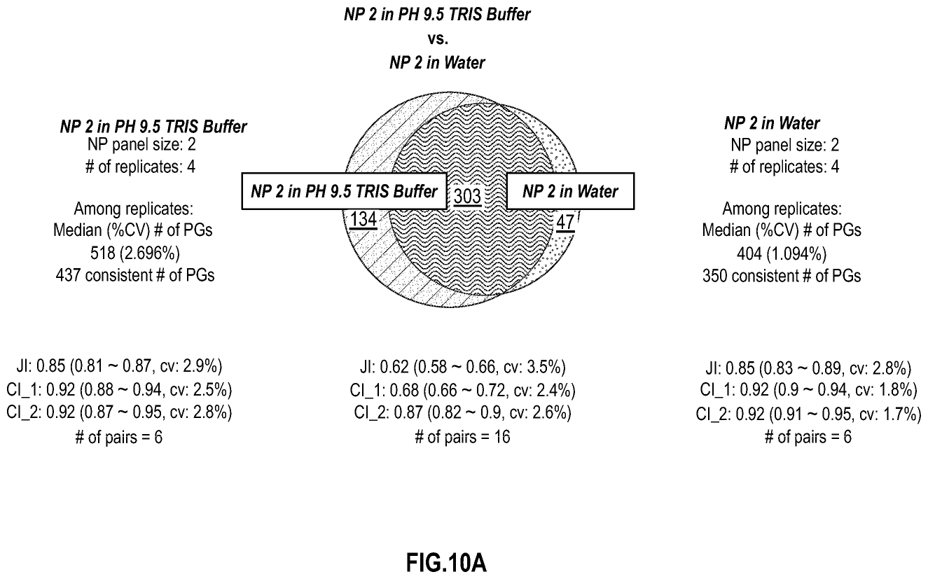

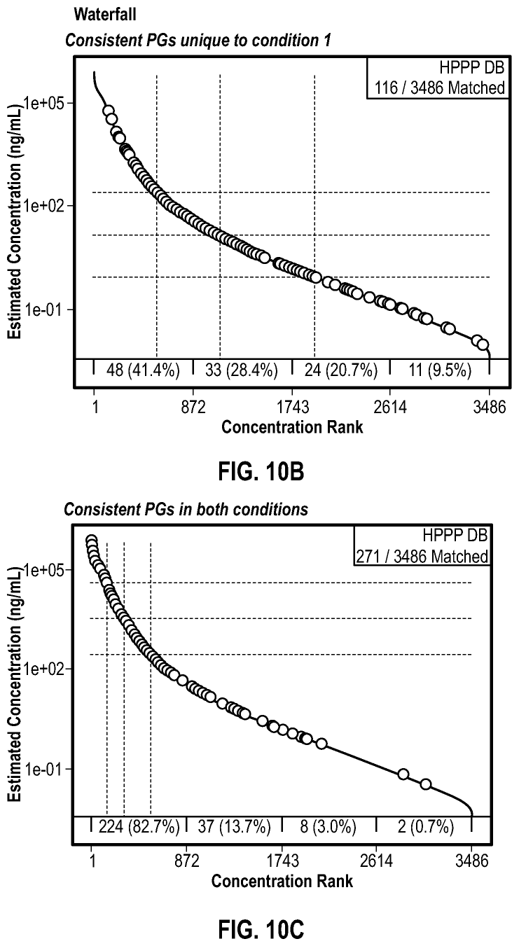

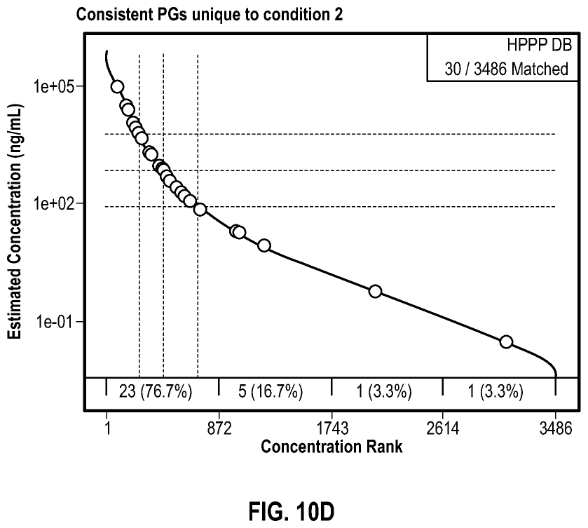

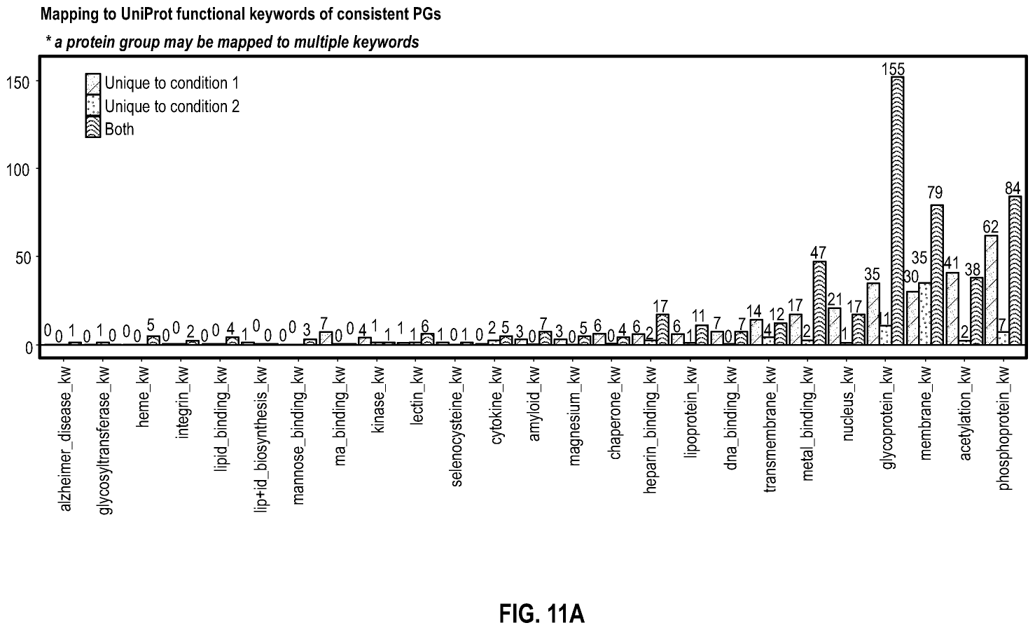

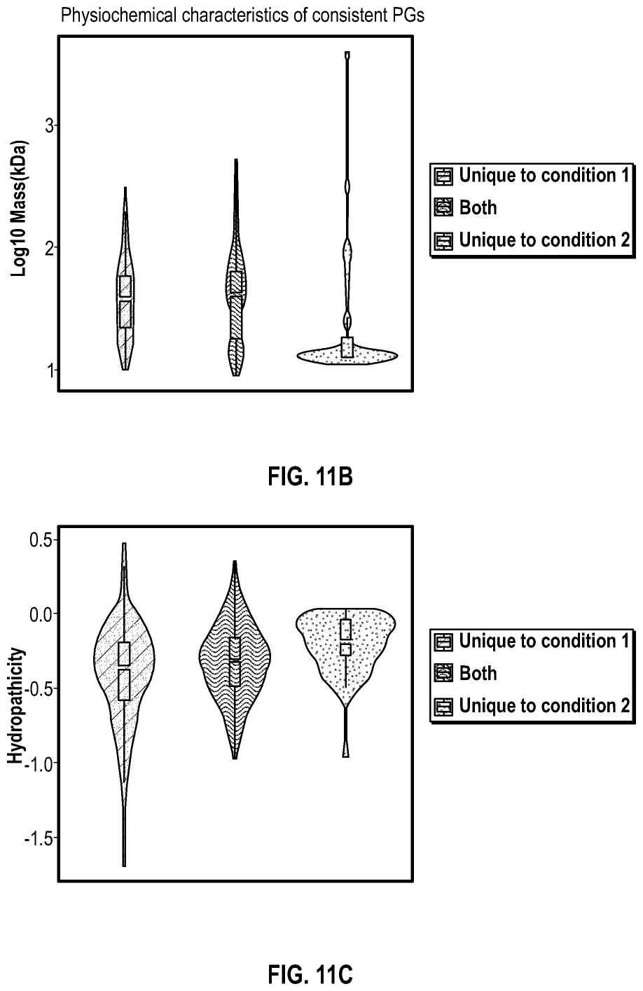

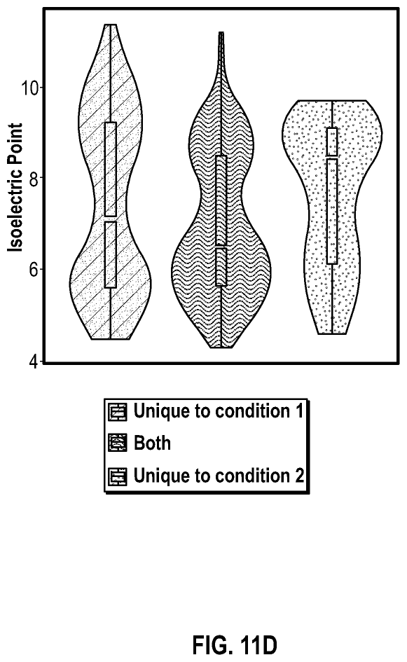

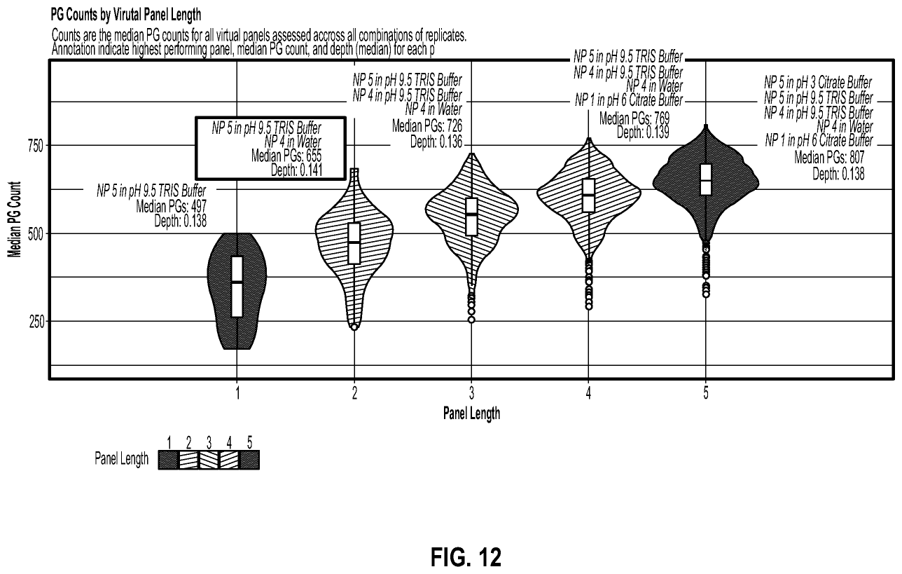

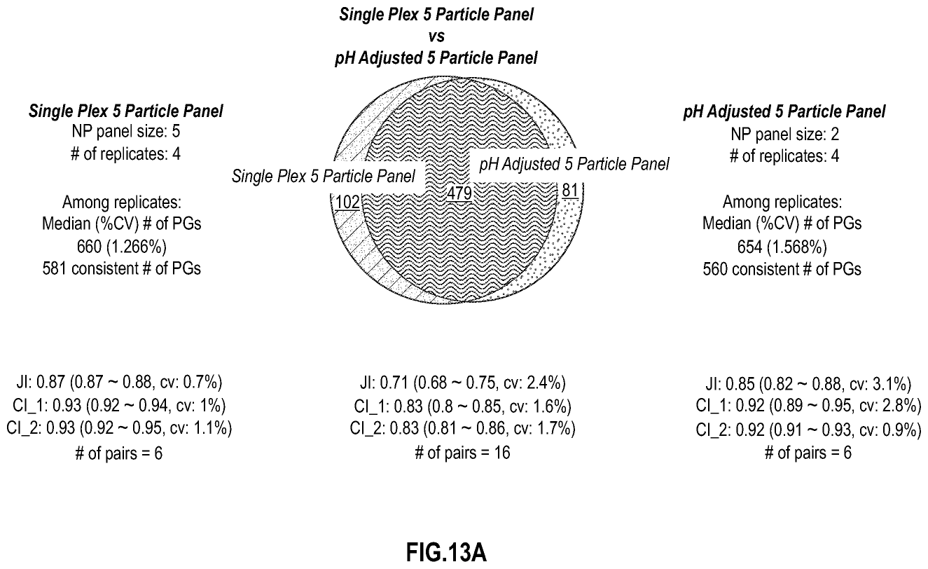

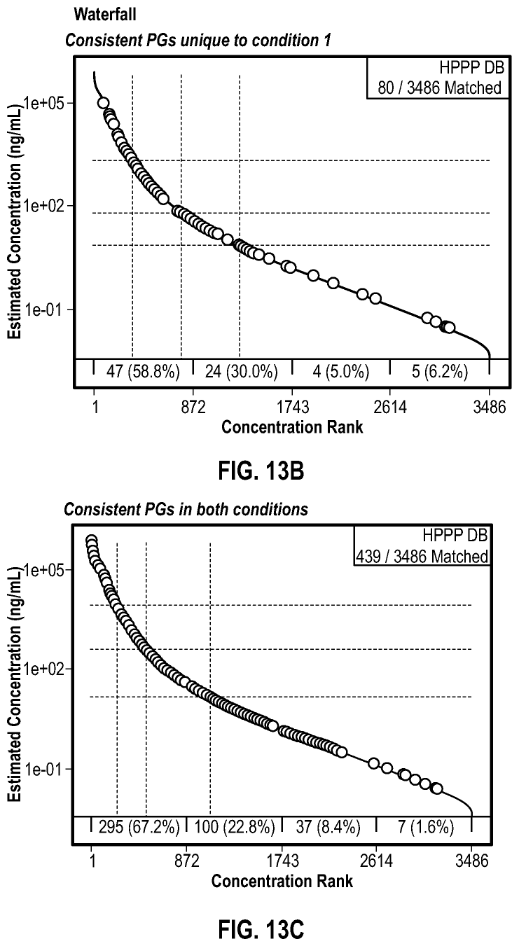

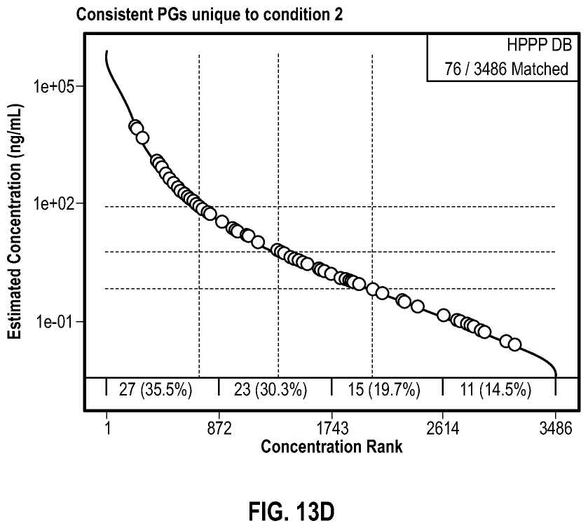

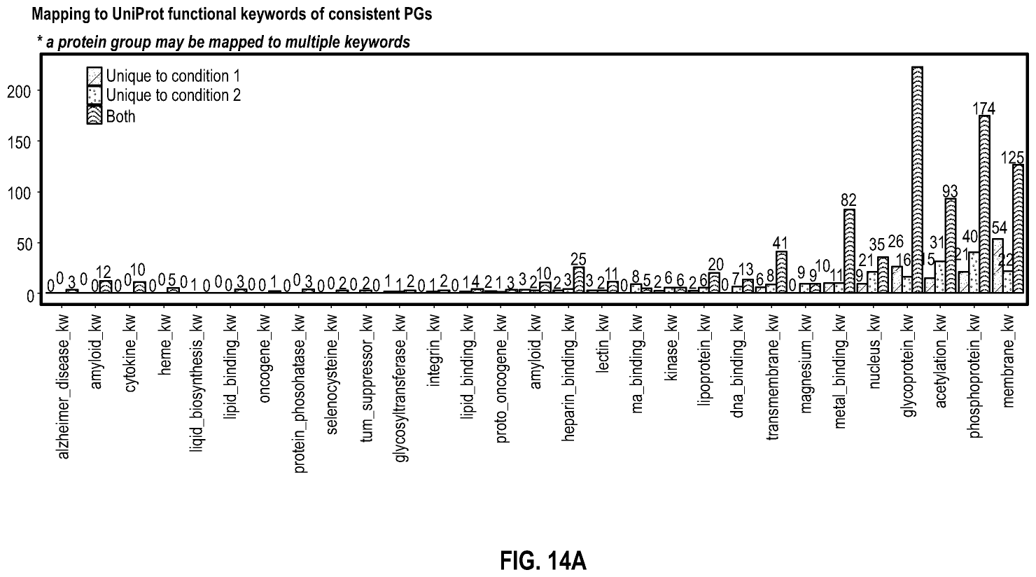

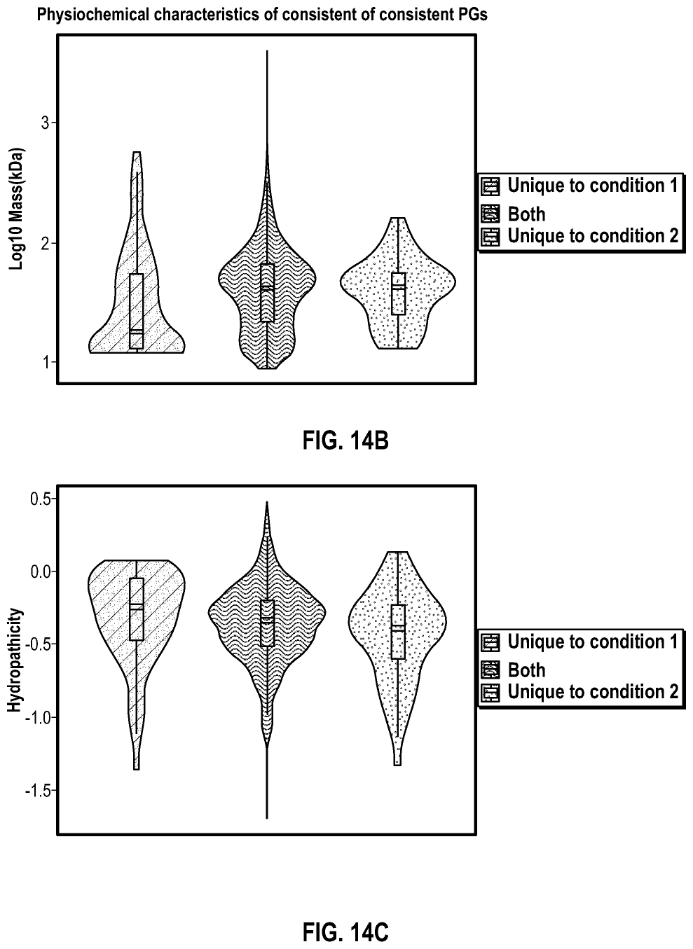

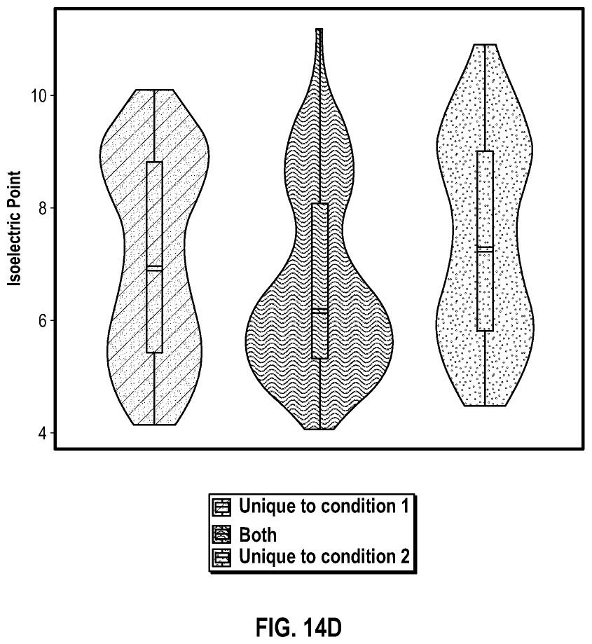

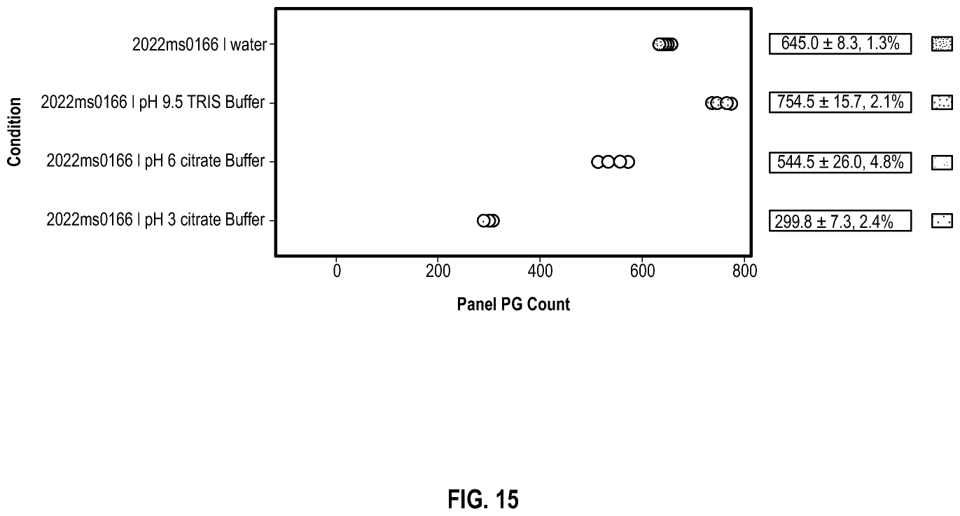

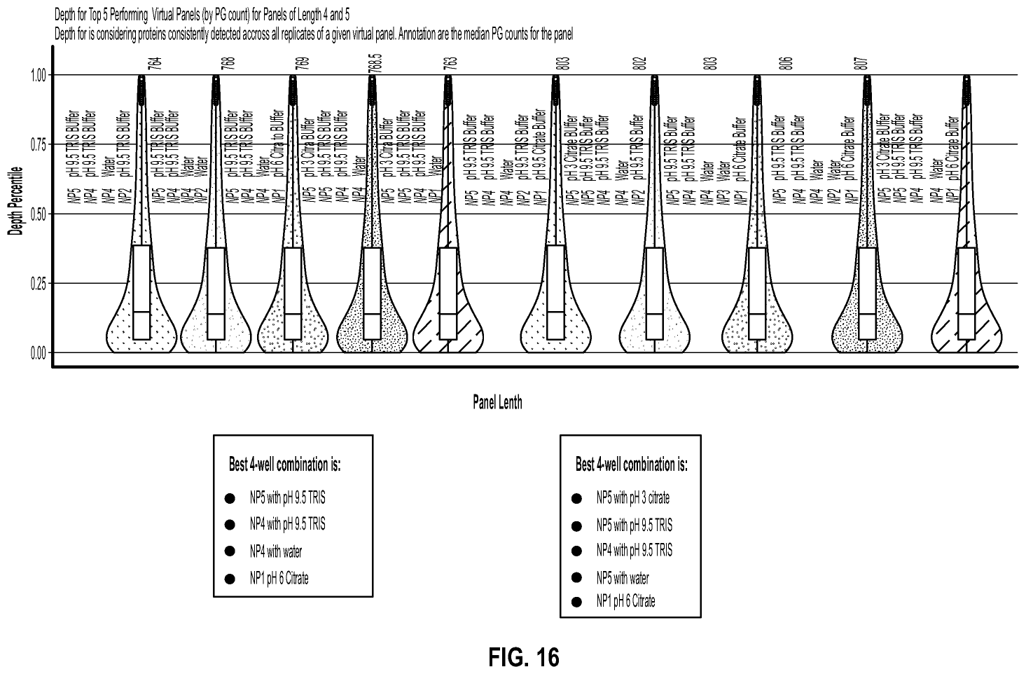

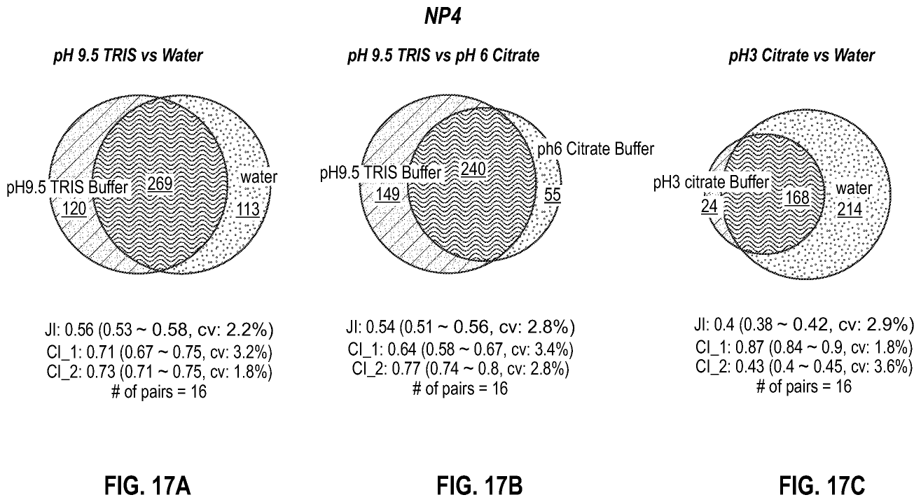

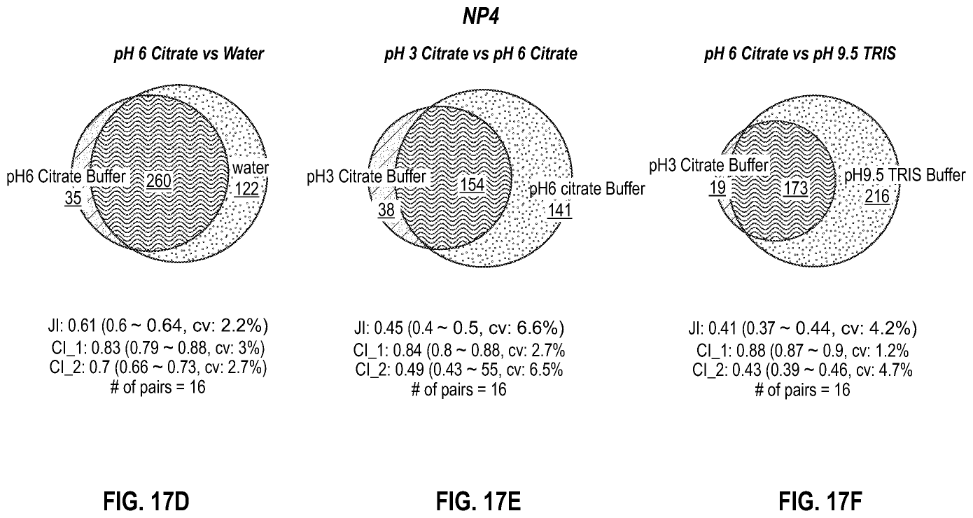

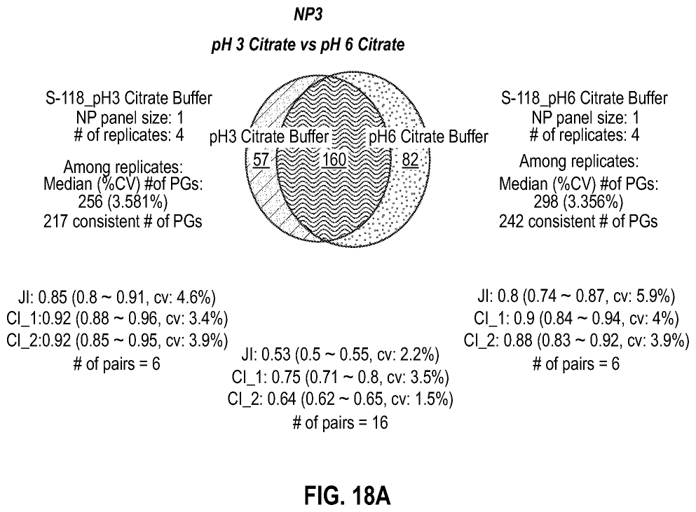

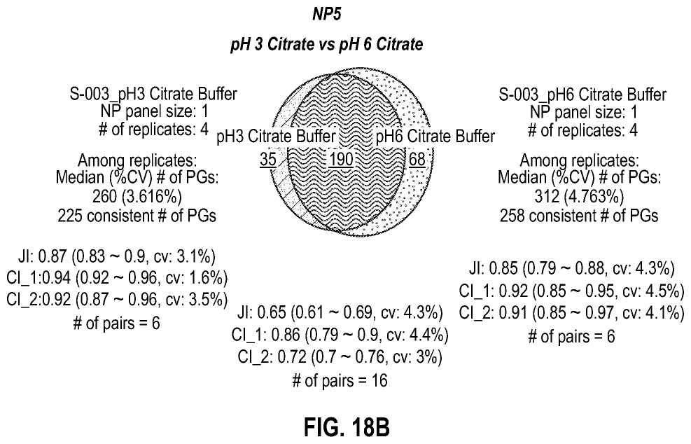

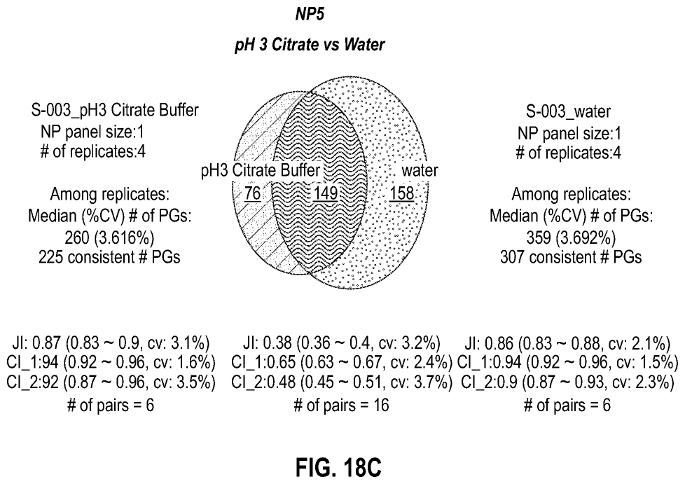

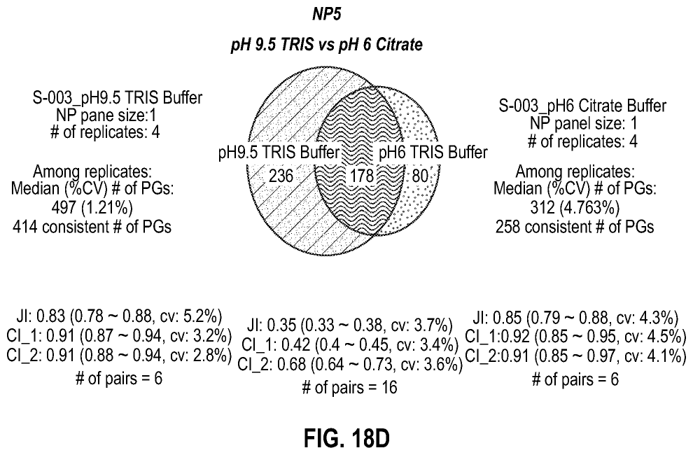

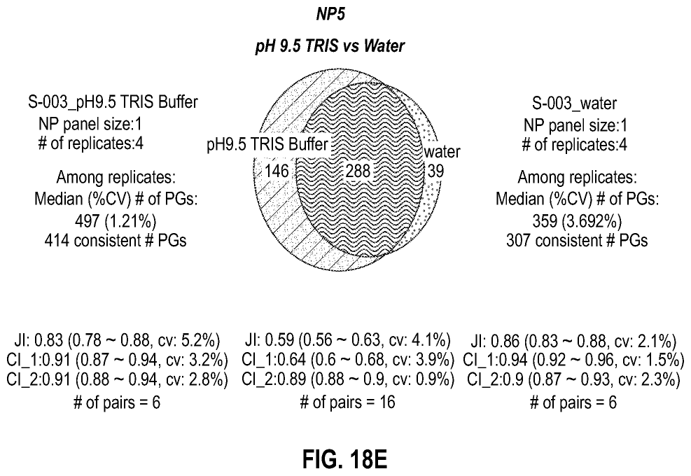

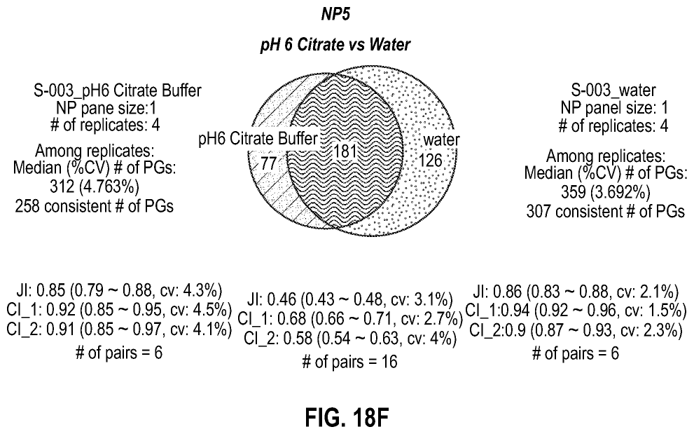

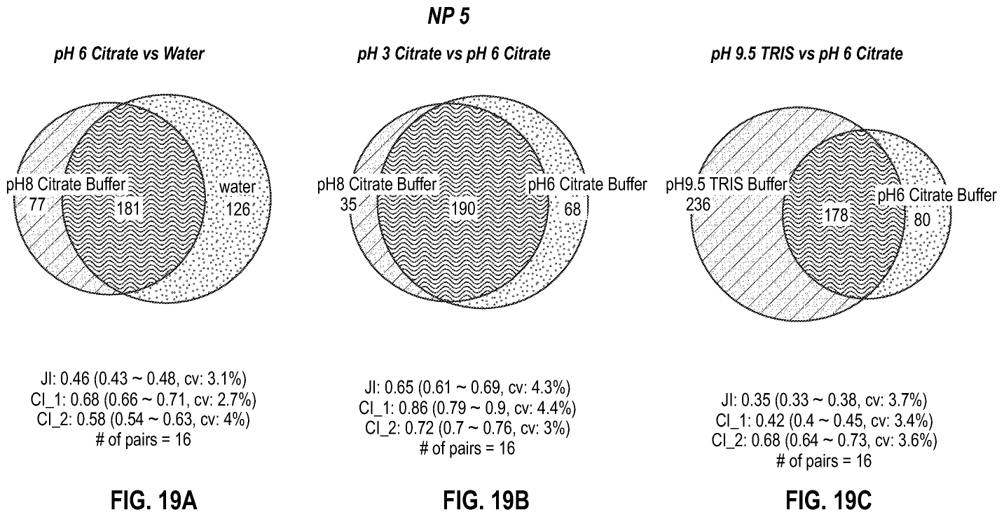

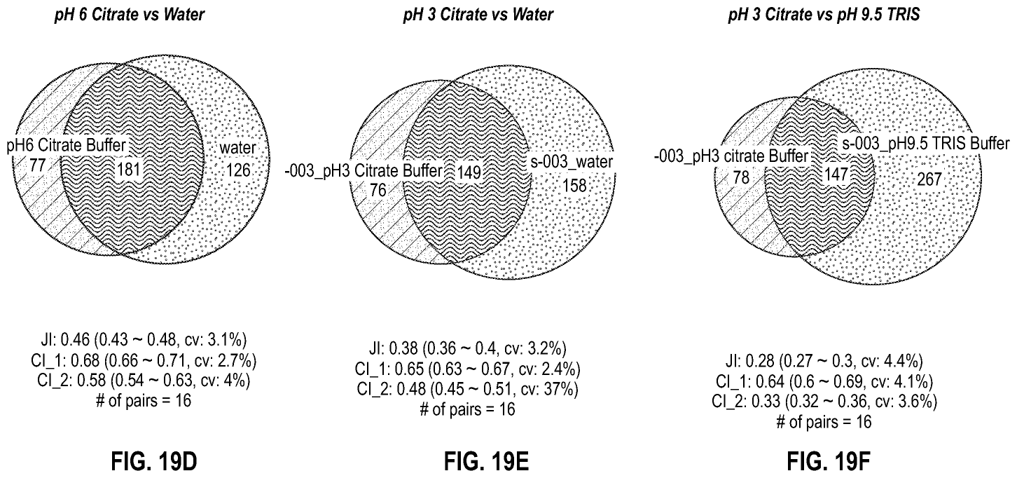

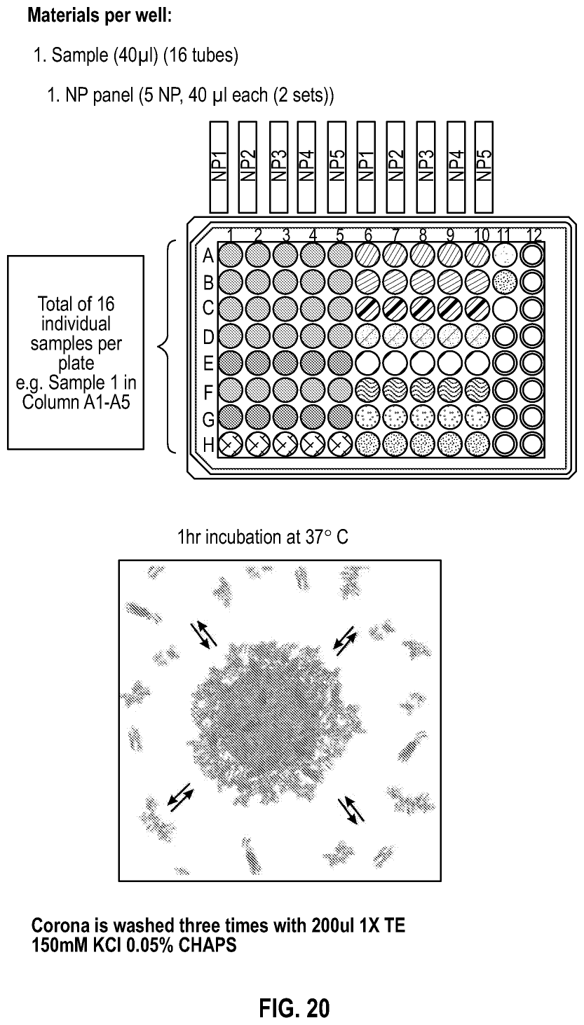

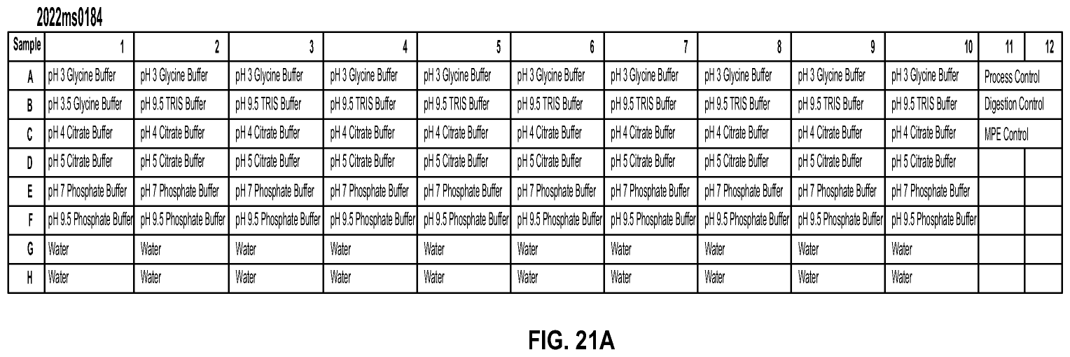

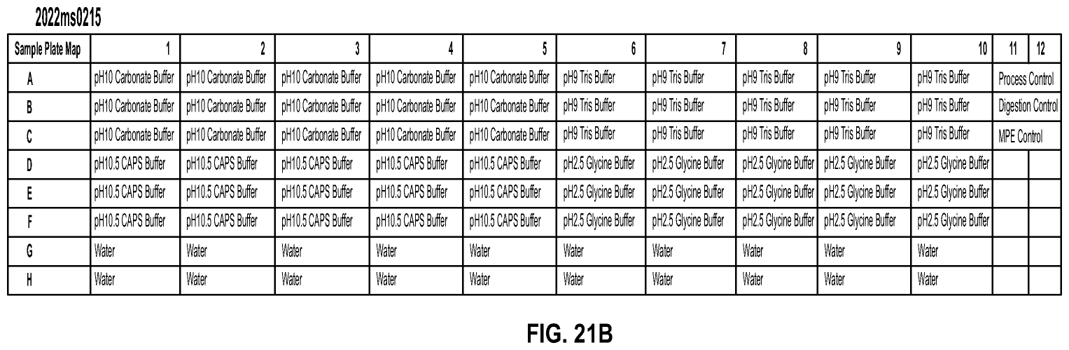

Low Jaccard Index (JI) was observed between pH of 7 (in water) and high pH (using TRIS buffer, pH=9.5) for NP 4, even though the number of protein groups were similar between the two conditions, as shown in FIG. 6A-6D. Functional annotations of the protein groups, shown in FIG. 7A, and the range of physicochemical properties of the proteins, shown in FIGS. 7B-7D, also varied significantly. The protein/NP ratio could be adjusted to further improve proteomic depth coverage (e.g., by utilizing the Vroman effect).

### Example 2: Protein Profiling of Biological Fluids with Proteograph™ Assay Using Multiplexed Nanoparticles

Nanoparticles may be incubated with a biological sample in isolation from one another, for example, in separate wells. To improve throughput for analyzing biological samples, it can be advantageous to incubate a given biological in fewer wells multiplexed with nanoparticles. This example provides a multiplexed approach to mix same charged NPs with different physicochemical properties to improve depth of proteome coverage and assay throughput. The multiplexed approach may incubate various NPs under different pH conditions.

Possible combinations of NPs with like charge (e.g., all positive or all negative zeta potential as measured by DLS) were explored, as well as the different ratios between the NPs of like charge. The combinations of like-charged NPs were multiplexed prior to incubating biosamples with the NPs. The performances of multiplexed NPs and single NP were compared.

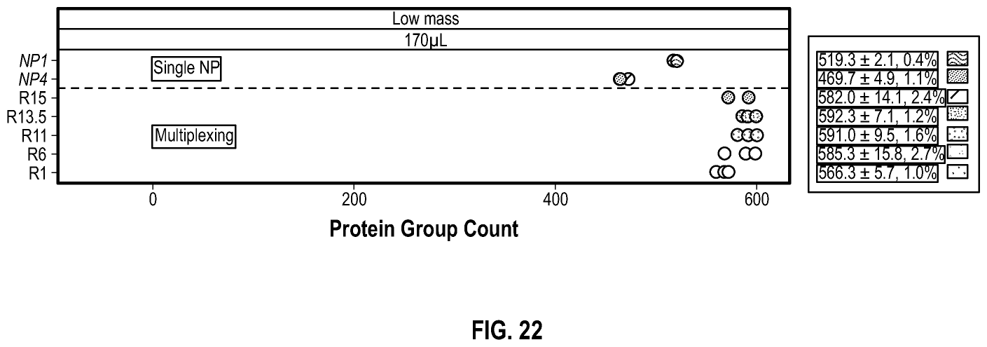

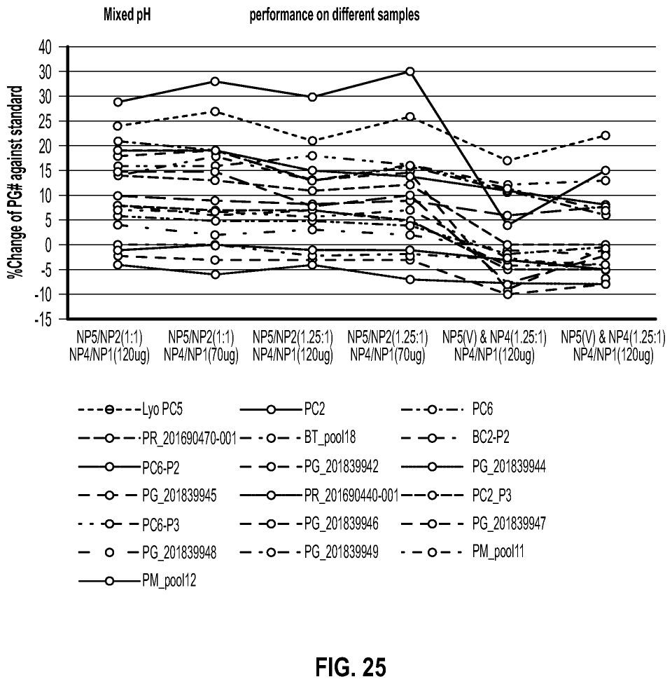

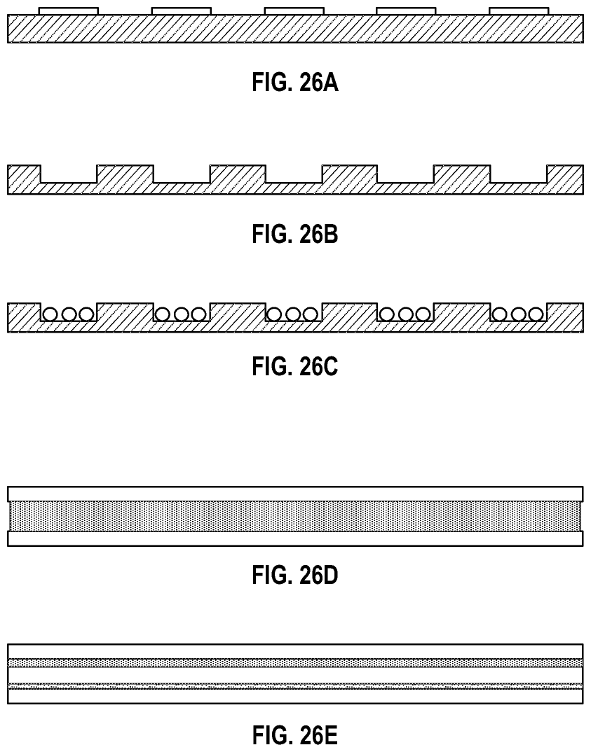

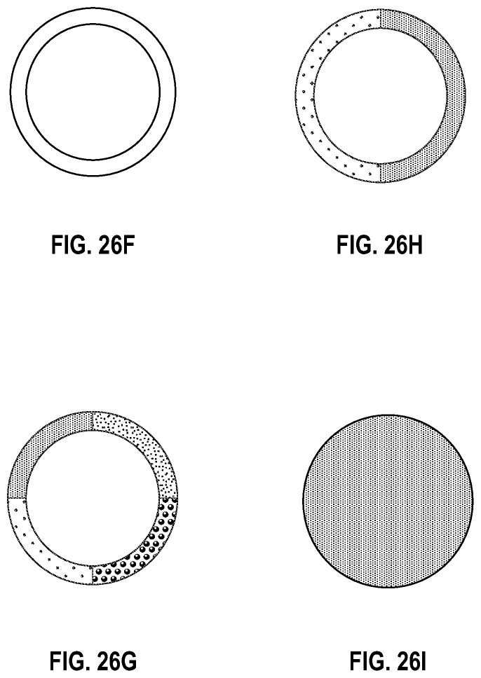

The results showed that by multiplexing multiple NPs into a single well, the number of protein groups identified were increased for many sample types, as shown in FIG. 22. By further incorporating high pH into this design, as shown in FIG. 25, a particle panel of 2 wells increased the number of protein groups identified by about 30% compared to a 5 particle panel performed with 5 wells. This also brings a 60% reduction in mass spec time per sample. A top performing 4-well or 5-well particle panel had mixed buffer conditions including pH 6, water, and pH 9.5.

FIG. 22 shows a comparison for the number of protein groups identified between a multiplexed assay compared to a single-plex assay, in accordance with some embodiments. Improved protein group numbers were observed with NP 1:NP 4 at a mass ratio of about 6 to 15.

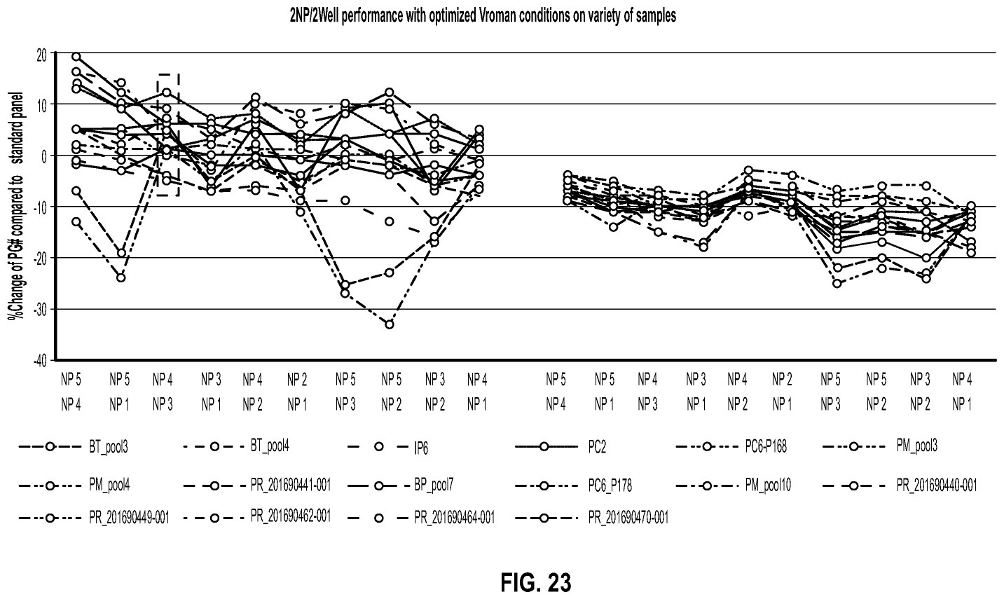

FIG. 23 shows a comparison for the number of protein groups identified between various multiplexed assays with different Vroman conditions, in accordance with some embodiments. The combinations had between-5% and +8% of the number of protein groups identified, when compared to a reference particle panel of 5 particles. Stable performance was observed for all samples when NP 4 and NP 1 were combined (as shown in the dotted box).

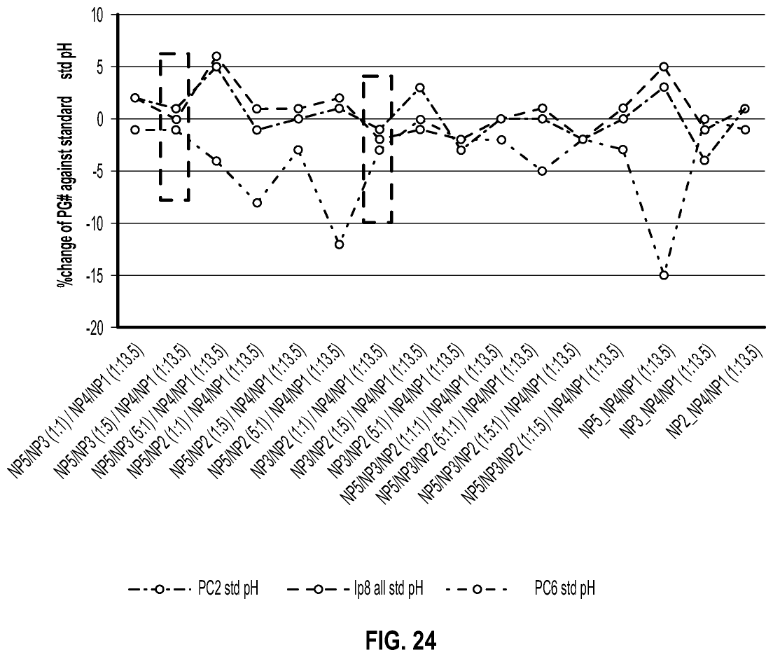

FIG. 24 shows a comparison for the number of protein groups identified between various multiplexed assays, in accordance with some embodiments. Multiplexed NPs with standard pH conditions performed better than single NP (dotted boxes compared to solid lined box).

FIG. 25 shows a comparison for the number of protein groups identified between various multiplexed assays with different pH conditions, in accordance with some embodiments. NP 5 and NP 2 were multiplexed at pH 9.5 while NP 4 and NP 1 were multiplexed at a standard pH. An average of +9% improvement than a reference 5 particle panel was observed for all sample types.

### Example 3: Protein Profiling of Biological Fluids with Proteograph™ Using Low Sample Volumes

Plasma samples were diluted 1:1 with (i) water, (ii) 1 M pH 9.5 Tris Buffer, (iii) pH 7.4 TE buffer, or (iv) phosphate buffered saline. These diluted plasma samples and an undiluted control plasma sample were processed in triplicate using the Proteograph™ and analyzed using LC-MS/MS. The total number of protein groups identified for each sample were as follows: control—1382.7±15.3; water—1156.7±9.0; pH 9.5 Tris buffer—1321.0±29.1; pH 7.4 TE—1084±2.8; and PBS—1188±5.7. While all dilutions reduced the total number of protein groups detected in the process plasma samples, pH 9.5 Tris buffer provided significantly higher protein groups counts compared to the other dilutions.

### Example 4: Comparison of Sample Dilutions when Protein Profiling of Biological Fluids with Proteograph™ Workflow

Different diluted volumes of pooled human plasma and mouse serum samples were analyzed in triplicate using the Proteograph™ v1.2 workflow. The sample volumes tested were 250 μL, 125 μL, 50 μL, 25 μL, and 10 μL. Sample volumes below 250 μL were diluted with 1 M pH 9.5 Tris Buffer to obtain a total volume of 250 μL. For example, 50 μL of the pooled plasma sample was diluted with 200 μL of 1 M pH 9.5 Tris Buffer. The final volumes were processed in triplicate using the Proteograph™ v1.2 workflow. Briefly, 40 μL volumes were aliquoted into 5 separate wells, and each well was combined with 40 μL of different nanoparticles suspended in water. The combined mixture was then incubated to form a biomolecule corona, followed by washing, digestion, and preparation for analysis by mass spectrometry. The processed peptide samples were analyzed using LC-MS/MS in DIA mode.

The number of protein groups and peptides identified in each sample are provided in Table 1 below:

| | | | Median | |
|---|---|---|---|---|
| Biological | Sample | Dilution | Protein Groups | Median Peptides |
| Sample | Volume | Ratio | Identified | Identified |
| Pooled Human | 250 μL | None | 2167 | 17007 |
| Plasma Sample | 125 μL | 1:1 | 2024 | 15886 |
| | 50 μL | 1:4 | 1701 | 13611 |
| | 25 μL | 1:9 | 1528 | 12503 |
| | 10 μL | 1:24 | 1278 | 10651 |
| Mouse Serum | 250 μL | None | 2918 | 20477 |
| | 125 μL | 1:1 | 2694 | 18789 |
| | 50 μL | 1:4 | 2485 | 16757 |
| | 25 μL | 1:9 | 2288 | 15409 |
| | 10 μL | 1:24 | 2013 | 13383 |

The results show that the number of protein groups and peptides identified decreases with dilution, but even when analyzing a 1:24 dilution of a 10 μL sample volume, a majority of the protein groups and peptides are identified relative to the undiluted sample. The coefficient of variance for each dilution was below 25%.

About 1060 protein groups were identified in the undiluted pooled human plasma samples (i.e. 250 μL plasma sample volume) that were also annotated in the Human Plasma Proteome Project (HPPP). These protein groups were sorted using abundance data from the HPPP and grouped into quartiles, with the first quartile containing the most abundant proteins and the fourth quartile containing the least abundant proteins. The number of proteins not identified in each quartile for each dilution compared to the undiluted 250 μL sample volume is shown in Table 2 below:

| | 250 μL v | 250 μL v | 250 μL v | 250 μL v |
|---|---|---|---|---|
| | 125 μL | 50 μL | 25 μL | 10 μL |
| # proteins w/o ID in 1st | 8 | 15 | 16 | 28 |
| Q | | | | |
| # proteins w/o ID in 2nd | 8 | 39 | 57 | 93 |
| Q | | | | |
| # proteins w/o ID in 3rd | 17 | 37 | 60 | 99 |
| Q | | | | |
| # proteins w/o ID in 4th | 9 | 30 | 44 | 68 |
| Q | | | | |
| Total | 42 | 121 | 177 | 288 |

The results show that the dilution reduces the number of protein groups identified in each quartile, with a greater loss for lower abundance protein groups. Even with a 1:24 dilution used for 10 μL plasma samples, the majority of protein groups were identified in the fourth quartile compared to the undiluted sample.

The Jaccard index was determined for each diluted plasma sample relative to the undiluted 250 μL, which is an indicator of the similarity of protein groups identified in between samples. The Jaccard index between the samples was: 250 μL v. 125 μL-0.93; 250 μL v. 50 μL-0.87; 250 μL v. 25 μL-0.83; and 250 μL v 10 μL-0.70. These results indicate that similar protein groups can be identified when diluting the samples.

### Example 5: System for Selectively Enriching and Assaying Biomolecules

This example illustrates use of an automated system (an instrument): a pipeline comprising providing various consumable materials (e.g., nanoparticle formulations, solvents, reagents, etc.), using an automated system to conduct assays, using a mass spectrometer to produce assay results, and then data analysis software to analyze the results and display results to a user.

The following describes an example method on an automated system comprising a computer readable medium comprising machine-executable code. (1) A user (i.e., an operator) prepares samples (e.g., by thawing frozen samples), reagents (e.g., diluting reagents), and particles (e.g., reconstituting lyophilized particles into a multiplexed particle suspension). The prepared samples, reagents, and particles are loaded into the automated system. The automated system then automatically carries out experimental steps from this point forward, including: (2) device initialization (Chassis, MPE2, Hamilton Heater Shaker (HHS), Inheco CPAC) that executed within 5 minutes, (3) Pipetting samples to assay plate executed within 5 minutes, (4) pipetting particles to assay plate executed within 15 minutes, (5) incubation at 37° C. executed within 60 minutes, (6) assay plate washing executed within 30 minutes, (7) addition of lysis, reduction, and alkylation buffer to assay plate executed within 10 minutes, (8) incubation at 95° C. executed on HHS within 10 minutes, (9) assay plate cool down at room temperature executed within 20 minutes, (10) addition of trypsin/LysC enzyme executed within 8 minutes, (11) incubation at 37° C. with HHS executed within 180 minutes, (12) addition of stop solution executed within 3 minutes, (13) pull down of particles executed within 5 minutes, (14) processing samples using SPE plate on MPE2 executed within 8 minutes, (15) processing samples with Wash A using SPE plate on MPE2 executed within 8 minutes, (16) processing samples with Wash B-1 using SPE plate on MPE2 executed within 8 minutes, (17) processing samples with Wash B-2 using SPE plate on MPE2 executed within 8 minutes, and (18) eluting samples using SPE plate on MPE2 executed within 5 minutes. (19) The user can then clean-up the automated system after the end of the experiment. The total duration of the experiment is about 7 hours.

The previously described series of experimental steps may include extra steps, may exclude some steps, or may have variations in each step. These variations may be implemented such that a user can select which variation is to be used. For example, there may be variations in step (1), wherein the user can dilute a sample (e.g., a plasma sample up to 20 times its original volume), select a different volume for the assay (e.g., anywhere from 40 μL to 100 μL), thaw a sample to a specific temperature (e.g., room temperature or 4° C.), single-plex or multiplex nanoparticles (e.g., 2, 3, 4, 5, or any number of particles per partition), or carry out interference steps on the sample (e.g., hemolysis/lipid concentration). In some cases, a background of biomolecules other than proteins may change protein coronas depending on the physicochemical properties of a particle. In some cases, the background of biomolecules may also form a part of a biomolecule corona. In some cases, an interference step may comprise titrating different concentrations of certain biomolecules (e.g., of lipids) at different concentration.

There may be variations in any of the incubations steps, wherein the duration of time for incubation can be varied (e.g., 5 min or overnight), the pH of the solution being incubated can be varied (e.g., pH of 3.8, 5.0, or 7.4), the ionic strength of the solution being incubated can be varied (e.g., 0, 50, or 150 mM), and the rate at which the solution being incubated is shaken can be varied (e.g., 0, 150, or 300 RPM).

There may be variations in any of the wash steps, wherein some or all of the constituents in a solution can be resuspended, or not resuspended. Some or all of the constituents in a solution can be separated, for example, by applying a magnetic field to capture magnetic particles.

There may be variations in the lysis, reduction, or alkylation steps, wherein a step-wise denaturation can take place. The temperature of the solution can be varied (e.g., 50° C. or 95° C.). There may be steps where proteins or peptides are digested, for example, by using trypsin at various concentrations (1×, 2× concentration of a standard amount of trypsin) for various durations of time (e.g., 3 hours or overnight). In some cases, standard amount for trypsin may range from about 1/10 to about 1/100 mass of trypsin compared to the mass of proteins. Proteins or peptides may be digested in a stepwise fashion, for example, by using Trypsin/LysC.

There may be variations in the elution step. The elution volume can be varied (e.g., 75, 150, or 300 μL), clean dry air (CDA) or nitrogen can be supplied at various pressures (anywhere from 0 to 50 psi), different types of solid phase extraction (SPE) plates may be used (e.g., Thermal Fisher SPE plates, iST, C18 or other substrates).

In some cases, the automated system can be configured run 8 to 16 samples at one time. Biomolecules in a biological sample (i.e., biofluid) can be measured with 5 different approaches per sample. Measurements can be conducted on multiple biofluids including plasma, cell extracts, and lysates. Measurements can be done automatically and be completed 7-8 hours, with peptides ready to be injected into liquid chromatography (LC) or MS for detection. Unbiased measurements allow for reduced LC/MS time, and these measurements can be agnostic of the LC/MS detector or approach, for instance: no more than 30 min gradient length (sample to sample) per fraction using DIA SWATH (data independent acquisition) approach on Sciex 6600+, and/or no more than 1 hour gradient length DDA (data dependent acquisition) approach on Thermo Orbitrap Lumos. DIA SWATH (data independent acquisition) and DDA (data independent acquisition) are modes for MS and differ in the ways that peptides are analyzed and the ways that proteins are computationally reconstructed based on the MS raw data. Because measurements can be done on intact proteins, the measurements may reveal protein-protein interactions in the experimental data.

In some cases, the automated system can comprise a 96 well plate that can accommodate up to 16 samples with 5 nanoparticles interrogation. In some cases, the amount of required sample volume can be less than or equal to 240 μL or 40 μL. In some cases, reagents can be stored while retaining stability for greater than 9 months at 4° C. or great than 6 months at room temperature. In some cases, the assay can run within 7 hours. In some cases, MS experiment run time can be within 120 minutes. In some cases, MS experiment may be run with ScanningSWATH. In some cases, ScanningSWATH can refer to a rapid MS acquisition mode for short gradients, down to a few minutes. In some cases, ScanningSWATH can refer to a rapid MS acquisition mode using a scanning quadrupole. In some cases, ScanningSWATH can use Sciex timsTOF rapid IMS-IMS, which can involve ion mobility separation and can involve upfront separation of ions based on their charge/dipole and shape properties. In some cases, the automated system can comprise analysis tools including visualization (e.g., group-analysis, PCA) tools or quality control tools, which may be integrated into a cloud-based computing system. In some cases, the protein detection method implemented on the automated system can show 5× superiority (i.e., superiority in the number of protein groups detected) over shallow plasma methods and 3× superiority over depleted plasma methods. In some cases, the protein detection method implemented on the automated system can have 5% improvement in precision (lower CV) over published datasets (e.g., Geyer et al. Mol. Syst. Biol. 13, 942 (2017).

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method comprising: (a) selectively enriching a first plurality of biomolecule types in a first fluid composition; (b) selectively enriching a second plurality of biomolecule types in a second fluid composition; and (c) performing a downstream assay on biomolecule types of the first plurality of biomolecule types and the second plurality of biomolecule types, wherein the first fluid composition comprises a first pH, and the second fluid composition comprises a second pH, wherein the first pH and the second pH are different or the same.

Embodiment 2. The method of Embodiment 1, wherein a second infinite-dilution limit enthalpy or free energy of solvation of a second biomolecule in the second subset of biomolecules is different when the second biomolecule is in the second fluid composition compared to when the second biomolecule is in the first fluid composition.

Embodiment 3. The method of Embodiment 1 or 2, wherein the first fluid composition comprises a first set of intensive physical properties that mediates selective enrichment of the first plurality of biomolecule types, and the second fluid composition comprises a second set of intensive physical properties that mediates selective enrichment of the second plurality of biomolecule types, wherein the first set of intensive physical properties and the second set of intensive physical properties are different.

Embodiment 4. The method of any one of Embodiments 1-3, wherein the first fluid composition comprises a first ratio between a first sample volume and a first surface area of the first surface, and the second fluid composition comprises a second ratio between a second sample volume and a second surface area of the second surface, wherein the first ratio and the second ratio are different.

Embodiment 5. The method of any one of Embodiments 1-4, wherein the first fluid composition and the second fluid composition comprise different temperatures.

Embodiment 6. The method of any one of Embodiments 1-5, wherein the first fluid composition comprises a first ionic strength and the second fluid composition comprises a second ionic strength, wherein the first ionic strength and the second ionic strength are different.

Embodiment 7. The method of any one of Embodiments 1-6, wherein the first fluid composition and the second fluid composition comprise different salts.

Embodiment 8. The method of any one of Embodiments 1-7, wherein the first fluid composition and the second fluid composition comprises different solvents.

Embodiment 9. The method of any one of Embodiments 1-8, wherein the first fluid composition, the second fluid composition, or both comprise a buffer comprising tris(hydroxymethyl) aminomethane.

Embodiment 10. The method of any one of Embodiments 1-9, wherein the first fluid composition, the second fluid composition, or both comprise a buffer comprising citrate.

Embodiment 11. The method of any one of Embodiments 1-10, wherein the first fluid composition, the second fluid composition, or both comprise a pH between 2 and 4.

Embodiment 12. The method of any one of Embodiments 1-11, wherein the first fluid composition, the second fluid composition, or both comprise a pH between 5 and 7.

Embodiment 13. The method of any one of Embodiments 1-12, wherein the first fluid composition, the second fluid composition, or both comprise a pH between 9 and 10.

Embodiment 14. The method of any one of Embodiments 1-13, wherein the first fluid composition, the second fluid composition, or both comprise a pH of at least about 1, 2, 3, 4, 5, 6, 7, 8, 9, 10, 11, 12, 13, or 14.

Embodiment 15. The method of any one of Embodiments 1-14, wherein the first fluid composition, the second fluid composition, or both comprise a pH of at most about 1, 2, 3, 4, 5, 6, 7, 8, 9, 10, 11, 12, 13, or 14.

Embodiment 16. The method of any one of Embodiments 1-15, further comprising diluting a sample to make the first composition, the second composition, or both.

Embodiment 17. The method of Embodiment 16, wherein the diluting comprises adding a buffer.

Embodiment 18. The method of Embodiment 17, wherein the buffer comprises a pH of at least about 1, 2, 3, 4, 5, 6, 7, 8, 9, 10, 11, 12, 13, or 14.

Embodiment 19. The method of Embodiment 17 or 18, wherein the buffer comprises a pH of at most about 1, 2, 3, 4, 5, 6, 7, 8, 9, 10, 11, 12, 13, or 14.

Embodiment 20. The method of any one of Embodiments 17-19, wherein the sample and the buffer comprises different pH.

Embodiment 21. The method of any one of Embodiments 16-20, wherein the sample comprises at most about 1,000, 100, 10, 1, 0.1, 0.01, or 0.001 nanograms of biomolecules.

Embodiment 22. The method of any one of Embodiments 16-21, wherein the sample comprises at most about 1,000, 100, 10, 1, 0.1, 0.01, or 0.001 nanograms of biomolecules per mL of the sample.

Embodiment 23. The method of any one of Embodiments 16-22, wherein the sample comprises biomolecules from at most about 1,000, 100, 10, or 1 cell.

Embodiment 24. The method of any one of Embodiments 16-23, wherein the sample comprises at most about 1,000, 100, 10, 1, 0.1, 0.01, or 0.001 microliters.

Embodiment 25. The method of any one of Embodiments 16-24, wherein the sample comprises a complex biological sample.

Embodiment 26. The method of Embodiment 25, wherein the complex biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 27. The method of Embodiment 26, wherein the biological sample comprises plasma.

Embodiment 28. The method of any one of Embodiments 1-27, wherein the selectively enriching the first plurality of biomolecule types in the comprises contacting the first fluid composition with a first surface to adsorb the first plurality of biomolecule types on the first surface.

Embodiment 29. The method of any one of Embodiments 1-28, wherein the selectively enriching the second plurality of biomolecule types in the comprises contacting the second fluid composition with a second surface to adsorb the second plurality of biomolecule types on the second surface.

Embodiment 30. The method of any one of Embodiments 1-29, wherein the first surface, the second surface, or both comprise a particle.

Embodiment 31. The method of Embodiment 30, wherein the particle is a porous particle.

Embodiment 32. The method of Embodiment 30 or 31, wherein the particle is a microparticle.

Embodiment 33. The method of Embodiment 30 or 31, wherein the particle is a nanoparticle.

Embodiment 34. The method of any one of Embodiments 30-33, wherein the particle comprises a paramagnetic material.

Embodiment 35. The method of Embodiment 34, wherein the paramagnetic material is a superparamagnetic material.

Embodiment 36. The method of Embodiment 34 or 35, wherein the paramagnetic material comprises iron oxide.

Embodiment 37. The method of any one of Embodiments 1-36, wherein the selectively enriching the first plurality of biomolecule types in the comprises contacting the first fluid composition with a first plurality of surface types to adsorb the first plurality of biomolecule types on the first plurality of surface types.

Embodiment 38. The method of any one of Embodiments 1-37, wherein the selectively enriching the second plurality of biomolecule types in the comprises contacting the second fluid composition with a second plurality of surface types to adsorb the second plurality of biomolecule types on the second plurality of surface types.

Embodiment 39. The method of any one of Embodiments 1-38, wherein the first plurality of surface types, the second plurality of surface types, or both comprise the same sign of charge.

Embodiment 40. The method of any one of Embodiments 1-39, wherein the first plurality of surface types, the second plurality of surface types, or both comprise the same sign of zeta potential.

Embodiment 41. The method of any one of Embodiments 1-40, wherein the first plurality of surface types, the second plurality of surface types, or both comprise an acidic functional group.

Embodiment 42. The method of any one of Embodiments 1-41, wherein the acidic functional group comprises a Bronsted-Lowry acid or a Lewis acid functional group.

Embodiment 43. The method of any one of Embodiments 1-42, wherein the first plurality of surface types, the second plurality of surface types, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

Embodiment 44. The method of any one of Embodiments 1-43, further comprising, before (c), washing the first plurality of biomolecule types with a first wash composition and washing the second plurality of biomolecule types with a second wash composition.

Embodiment 45. The method of Embodiment 44, wherein the first fluid composition and the first wash composition comprise at least one common intensive physical property.

Embodiment 46. The method of Embodiment 44 or 45, wherein the first fluid composition and the first wash composition comprise at least one common solvent.

Embodiment 47. The method of any one of Embodiments 44-46, wherein the first fluid composition and the first wash composition comprise at least one different intensive physical property.

Embodiment 48. The method of any one of Embodiments 44-47, wherein the second fluid composition and the second wash composition comprise at least one common intensive physical property.

Embodiment 49. The method of any one of Embodiments 44-48, wherein the second fluid composition and the second wash composition comprise at least one common solvent.

Embodiment 50. The method of any one of Embodiments 44-49, wherein the second fluid composition and the second wash composition comprise at least one different intensive physical property.

Embodiment 51. The method of any one of Embodiments 44-50, wherein the first wash composition and the second wash composition comprise at least one common intensive physical property.

Embodiment 52. The method of any one of Embodiments 44-51, wherein the first wash composition and the second wash composition are the same.

Embodiment 53. The method of any one of Embodiments 44-52, wherein the first wash composition and the second wash composition comprises at least one different intensive physical property.

Embodiment 54. The method of any one of Embodiments 44-53, wherein the first wash composition releases the first plurality of biomolecule types adsorbed on the first surface from the first surface.

Embodiment 55. The method of Embodiment 54, further comprising purifying the first plurality of biomolecule types to produce a first purified composition.

Embodiment 56. The method of Embodiment 55, wherein the purifying comprises drying the plurality of biomolecule types to remove the first wash composition.

Embodiment 57. The method of Embodiment 56, further comprising reconstituting the first purified composition with a first reconstitution composition to produce a first reconstituted composition.

Embodiment 58. The method of any one of Embodiments 44-57, wherein the second wash composition releases the second plurality of biomolecule types deposited on the second surface from the second surface.

Embodiment 59. The method of Embodiment 58, further comprising purifying the second plurality of biomolecule types to produce a second purified composition.

Embodiment 60. The method of Embodiment 59, wherein the purifying comprises drying the second plurality of biomolecule types to remove the second wash composition.

Embodiment 61. The method of Embodiment 60, further comprising reconstituting the second purified composition with a second reconstitution composition to produce a second reconstituted composition.

Embodiment 62. The method of any one of Embodiments 1-61, wherein the downstream assay comprises mass spectrometry.

Embodiment 63. The method of Embodiment 62, wherein the mass spectrometry comprises LC-MS/MS.

Embodiment 64. The method of any one of Embodiments 1-63, wherein the downstream assay comprises protein sequencing.

Embodiment 65. The method of any one of Embodiments 1-64, wherein the downstream assay comprises contacting the first plurality of biomolecule types, the second plurality of biomolecule types, or both with a pair of antibodies capable of binding to at least one biomolecule type in the first plurality of biomolecule types, the second plurality of biomolecule types, or both, wherein the pair of antibodies comprises complementary single-stranded nucleic acid sequences attached thereto, such that when the pair of antibodies bind to the at least one biomolecule type, the complementary nucleic acids hybridize to form a double stranded nucleic acid, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

Embodiment 66. The method of any one of Embodiments 1-65, wherein the downstream assay comprises contacting the first plurality of biomolecule types, the second plurality of biomolecule types, or both with one or more aptamers capable of binding to at least one biomolecule type in the first plurality of biomolecule types, the second plurality of biomolecule types, or both, wherein the one or more aptamers are coupled to a surface via a cleavable linker.

Embodiment 67. The method of Embodiment 66, wherein the surface is a particle surface.

Embodiment 68. The method of Embodiment 67, wherein the cleavable linker is photocleavable.

Embodiment 69. The method of Embodiment 68, further comprising contacting the first plurality of biomolecule types, the second plurality of biomolecule types, or both with a macromolecular competitor configured to, in a fluid composition, reduce dissociation of a complex comprising the one or more aptamers and the first plurality of biomolecule types, the second plurality of biomolecule types, or both.

Embodiment 70. The method of Embodiment 69, wherein the macromolecular competitor is further configured to bind to a biomolecule type that is different from the first plurality of biomolecule types, the second plurality of biomolecule types, or both.

Embodiment 71. The method of Embodiment 70, wherein the macromolecular competitor is a polyanionic macromolecule.

Embodiment 72. The method of any one of Embodiments 1-71, wherein the downstream assay comprises nucleic acid sequencing.

Embodiment 73. The method of any one of Embodiments 1-72, wherein (a) and (b) are performed on one machine.

Embodiment 74. The method of any one of Embodiments 1-72, wherein (a) and (b) are performed on different machines.

Embodiment 75. The method of any one of Embodiments 1-74, wherein (a) and (b) are performed in parallel.

Embodiment 76. The method of any one of Embodiments 1-74, wherein (a) and (b) are performed in series.

Embodiment 77. The method of any one of Embodiments 1-76, wherein the first fluid composition and the second fluid composition are a portion of the same sample.

Embodiment 78. The method of any one of Embodiments 1-76, wherein the first fluid composition and the second fluid composition are from different samples.

Embodiment 79. The method of any one of Embodiments 1-78, wherein the selectively enriching is performed for the first fluid composition and the second fluid composition in parallel.

Embodiment 80. The method of any one of Embodiments 1-78, wherein the selectively enriching is performed for the first fluid composition and the second fluid composition in serial.

Embodiment 81. The method of any one of Embodiments 1-80, wherein the selectively enriching is performed at a rate of at least 100, 1,000, 10,000, 100,000, or 1,000,000 biomolecules enriched per hour.

Embodiment 82. The method of any one of Embodiments 1-81, wherein the selectively enriching is performed at a rate of at least 1, 10, 100, 1,000, 10,000, 100,000, 1,000,000, or 10,000,000 μg of biomolecules enriched per hour.

Embodiment 83. The method of any one of Embodiments 1-82, wherein the contacting is performed for the first fluid composition and the second fluid composition in parallel.

Embodiment 84. The method of any one of Embodiments 1-82, wherein the contacting is performed for the first fluid composition and the second fluid composition in serial.

Embodiment 85. The method of any one of Embodiments 1-84, wherein the contacting is performed at a rate of at least 100, 1,000, 10,000, 100,000, or 1,000,000 biomolecules deposited per hour.

Embodiment 86. The method of any one of Embodiments 1-85, wherein the contacting is performed at a rate of at least 1, 10, 100, 1,000, 10,000, 100,000, 1,000,000, or 10,000,000 μg of biomolecules deposited per hour.

Embodiment 87. The method of any one of Embodiments 1-86, wherein the washing is performed for the first fluid composition and the second fluid composition in parallel.

Embodiment 88. The method of any one of Embodiments 1-86, wherein the washing is performed for the first fluid composition and the second fluid composition in serial.

Embodiment 89. The method of any one of Embodiments 1-88, wherein the washing is performed at a rate of at least 100, 1,000, 10,000, 100,000, or 1,000,000 biomolecules washed per hour.

Embodiment 90. The method of any one of Embodiments 1-89, wherein the washing is performed at a rate of at least 1, 10, 100, 1,000, 10,000, 100,000, 1,000,000, or 10,000,000 μg of biomolecules washed per hour.

Embodiment 91. The method of any one of Embodiments 1-90, wherein the purifying is performed for the first fluid composition and the second fluid composition in parallel.

Embodiment 92. The method of any one of Embodiments 1-90, wherein the purifying is performed for the first fluid composition and the second fluid composition in serial.

Embodiment 93. The method of any one of Embodiments 1-92, wherein the purifying is performed at a rate of at least 100, 1,000, 10,000, 100,000, or 1,000,000 purified per hour.

Embodiment 94. The method of any one of Embodiments 1-92, wherein the purifying is performed at a rate of at least 1, 10, 100, 1,000, 10,000, 100,000, 1,000,000, or 10,000,000 μg of biomolecules purified per hour.

Embodiment 95. The method of any one of Embodiments 1-94, wherein the reconstituting is performed for the first fluid composition and the second fluid composition in parallel.

Embodiment 96. The method of any one of Embodiments 1-94, wherein the reconstituting is performed for the first fluid composition and the second fluid composition in serial.

Embodiment 97. The method of any one of Embodiments 1-96, wherein the reconstituting is performed at a rate of at least 100, 1,000, 10,000, 100,000, or 1,000,000 reconstituted per hour.

Embodiment 98. The method of any one of Embodiments 1-97, wherein the reconstituting is performed at a rate of at least 1, 10, 100, 1,000, 10,000, 100,000, 1,000,000, or 10,000,000 μg of biomolecules reconstituted per hour.

Embodiment 99. The method of any one of Embodiments 1-98, wherein the downstream assay is performed for the first fluid composition and the second fluid composition in parallel.

Embodiment 100. The method of any one of Embodiments 1-98, wherein the downstream assay is performed for the first fluid composition and the second fluid composition in serial.

Embodiment 101. The method of any one of Embodiments 1-100, wherein the downstream assay is performed at a rate of at least 1, 2, 3, 4, 5, 6, 7, 8, 9, 10, 100 samples assayed per hour.

Embodiment 102. The method of any one of Embodiments 1-101, wherein the downstream assay is performed at a rate of at least 1, 10, 100, 1,000, 10,000, 100,000, 1,000,000, or 10,000,000 μg of biomolecules assayed per hour.

Embodiment 103. The method of any one of Embodiments 1-102, wherein the downstream assay is performed at a rate of at least 100, 1,000, 10,000, 100,000, or 1,000,000 biomolecules identified per hour.

Embodiment 104. The method of any one of Embodiments 1-103, wherein the downstream assay is performed at a rate of at least 100, 1,000, 10,000, 100,000, or 1,000,000 protein groups identified per hour.

Embodiment 105. The method of any one of Embodiments 1-104, wherein the method is performed for the first fluid composition and the second fluid composition in parallel.

Embodiment 106. The method of any one of Embodiments 1-105, wherein the method is performed for the first fluid composition and the second fluid composition in serial.

Embodiment 107. The method of any one of Embodiments 1-106, wherein the method is performed at a rate of at least 1, 2, 3, 4, 5, 6, 7, 8, 9, 10, 100 samples per hour.

Embodiment 108. The method of any one of Embodiments 1-107, wherein the method is performed at a rate of at least 1, 10, 100, 1,000, 10,000, 100,000, 1,000,000, or 10,000,000 μg of biomolecules assayed per hour.

Embodiment 109. The method of any one of Embodiments 1-108, wherein the method is performed at a rate of at least 100, 1,000, 10,000, 100,000, or 1,000,000 biomolecules identified per hour.

Embodiment 110. The method of any one of Embodiments 1-109, wherein the method is performed at a rate of at least 100, 1,000, 10,000, 100,000, or 1,000,000 protein groups identified per hour.

Embodiment 111. The method of any one of Embodiments 1-110, wherein the downstream assay identifies at least about 100, 1,000, 10,000, 100,000, or 1,000,000 biomolecules when the first fluid composition and the second fluid composition is a HeLa cell extract

Embodiment 112. The method of any one of Embodiments 1-111, wherein the first plurality of biomolecule types and the second plurality of biomolecule types together comprise at least about 6, 7, 8, 9, 10, 11, 12, 13, 14, 15, or 16 dynamic range when the downstream assay is performed on the first fluid composition and the second fluid composition before the selectively enriching in (a) and (b).

Embodiment 113. The method of any one of Embodiments 1-112, wherein the first plurality of biomolecule types is depleted in at least a first biomolecule and enriched in a second biomolecule, wherein the first biomolecule is more abundant than the second biomolecule in the first fluid composition.

Embodiment 114. The method of any one of Embodiments 1-113, wherein the second plurality of biomolecule types is depleted in at least a first biomolecule and enriched in a second biomolecule, wherein the first biomolecule is more abundant than the second biomolecule in the second fluid composition.

Embodiment 115. The method of any one of Embodiments 1-114, wherein a first likelihood that the downstream assay identifies a low abundance biomolecule in the first plurality of biomolecule types or the second plurality of biomolecule types is larger than a second likelihood that the downstream assay identifies the low abundance biomolecule in the first fluid composition or the second fluid composition, wherein the low abundance biomolecule comprises less than about 1, 10⁻¹, 10⁻², 10⁻³, 10⁻⁴, 10⁻⁵, 10⁻⁶, 107, 10⁻⁸, 10⁻⁹, or 10⁻¹⁰ percent by mass of the first set of biomolecules or the second set of biomolecules.

Embodiment 116. The method of Embodiment 115, wherein the first likelihood is larger than the second likelihood by a factor of at least 2, 5, 10, 102, 102, 103, 104, 105, 106, 107, 108, 109, or 1010.

Embodiment 117. The method of any one of Embodiments 1-116, wherein the downstream identifies one or more biomolecules in the first plurality of biomolecule types, the second plurality of biomolecule types, or both, with a coefficient of variance of at most about 30, 25, 20, 15, 10, 5, 4, 3, 2, or 1 percent.

Embodiment 118. The method of any one of Embodiments 1-117, wherein the downstream identifies one or more biomolecules in the first plurality of biomolecule types, the second plurality of biomolecule types, or both, with a coefficient of variance of at least about 30, 25, 20, 15, 10, 5, 4, 3, 2, or 1 percent.

Embodiment 119. The method of Embodiment 117 or 118, wherein the one or more biomolecules comprise at least about 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, or 100,000 peptides.

Embodiment 120. The method of any one of Embodiments 117-119, wherein the one or more biomolecules comprise at least about 100, 200, 300, 400, 500, 600, 700, 800, 900, 1,000, 2,000, 3,000, 4,000, 5,000, 6,000, 7,000, 8,000, 9,000, or 10,000 proteins.

Embodiment 121. The method of any one of Embodiments 1-120, wherein the first plurality of biomolecule types, the second plurality of biomolecule types, or both comprise one or more polyamino acids.

Embodiment 122. The method of any one of Embodiments 1-121, further comprising identifying a biological state associated with the first fluid composition based at least partially on one or more physical properties of the first fluid composition.

Embodiment 123. The method of any one of Embodiments 1-122, further comprising identifying a biological state associated with the second fluid composition based at least partially on one or more physical properties of the second fluid composition.

Embodiment 124. The method of any one of Embodiments 44-123, further comprising identifying a biological state associated with the first fluid composition based at least partially on one or more physical properties of the first wash composition.

Embodiment 125. The method of any one of Embodiments 44-124, further comprising identifying a biological state associated with the second fluid composition based at least partially on one or more physical properties of the second wash composition.

Embodiment 126. The method of any one of Embodiments 57-125, further comprising identifying a biological state associated with the first fluid composition based at least partially on one or more physical properties of the first reconstitution composition.

Embodiment 127. The method of any one of Embodiments 61-126, further comprising identifying a biological state associated with the second composition based at least partially on one or more physical properties of the second reconstitution composition.

Embodiment 128. The method of any one of Embodiments 1-127, wherein the first surface is disposed in a first lyophilized composition comprising (i) at least one nanoparticle, and (ii) one or more modifiers.

Embodiment 129. The method of any one of Embodiments 1-128, wherein the second surface is disposed in a second lyophilized composition comprising (i) at least one nanoparticle, and (ii) one or more modifiers.

Embodiment 130. The method of Embodiment 128 or 129, wherein the one or more modifiers comprise pH modifiers, ionic strength modifiers, viscosity modifiers, or any combination thereof.

Embodiment 131. The method of any one of Embodiments 1-130, wherein the first pH and the second pH are different.

Embodiment 132. The method of any one of Embodiments 1-130, wherein the first pH and the second pH are the same.

Embodiment 133. A method comprising: (a) selectively enriching a first plurality of biomolecule types in a first fluid composition; (b) selectively enriching a second plurality of biomolecule types in a second fluid composition; and (c) performing a downstream assay on biomolecule types of the first plurality of biomolecule types and the second plurality of biomolecule types, wherein the first fluid composition and the second fluid composition are different such that a first infinite-dilution limit enthalpy or free energy of solvation of a first biomolecule in the first plurality of biomolecule types is different when the first biomolecule is in the first fluid composition compared to when the biomolecule is in the second fluid composition.

Embodiment 134. A kit comprising: (a) a first reagent comprising a first pH; (b) a second reagent comprising a second pH; (c) a first surface configured to selectively enrich a first plurality of biomolecule types in a first fluid composition comprising the first surface and the first reagent; and (d) a second surface configured to selectively enrich a second plurality of biomolecule types in a second fluid composition comprising the second surface and the second reagent, wherein the first fluid composition comprises the first reagent, and the second fluid composition comprises the second reagent, wherein the first pH and the second pH are different or the same.

Embodiment 135. A composition comprising: a suspension comprising: i. a first particle comprising (i) a first paramagnetic portion and (ii) a first surface chemistry; ii. a second particle comprising (i) a second paramagnetic portion and (ii) a second surface chemistry, wherein the second surface chemistry and the first surface chemistry are different; and iii. a plurality of biomolecules adsorbed on the first particle and the second particle.

Embodiment 136. The composition of Embodiment 135, wherein the first particle and the second particle comprise the same sign of charge.

Embodiment 137. The composition of Embodiment 135 or 136, wherein the first particle and the second particle comprise the same sign of zeta potential.

Embodiment 138. The composition of Embodiment 136 or 137, wherein the same sign is a negative sign.

Embodiment 139. The composition of any one of Embodiments 135-138, wherein the first surface chemistry, the second surface chemistry, or both comprise an acidic functional group.

Embodiment 140. The composition of Embodiment 139, wherein the acidic functional group comprises a Bronsted-Lowry acid or a Lewis acid functional group.

Embodiment 141. The composition of any one of Embodiments 135-140, wherein the first surface chemistry, the second first surface chemistry, or both comprise a carboxylate group, an acrylate group, a methacrylate group, an acetal group, a hemiacetal group, a hemiketal group, a sulfonic acid group, a sulfinic acid group, a thiocarboxylic acid group, a phosphonic acid group, a phosphate group, a phosphodiester group, a boronic acid group, a boronic ester group, a borinic acid group, a borinic ester group, silica group, a silanol group, a polymer, or any combination thereof.

Embodiment 142. The composition of any one of Embodiments 135-141, wherein the suspension is stable for at least about 1, 5, 10, 15, 30, or 60 minutes.

Embodiment 143. The composition of any one of Embodiments 135-142, wherein a time constant for destabilization of the suspension is at least about 1, 5, 10, 15, 30, or 60 minutes.

Embodiment 144. The composition of any one of Embodiments 135-143, wherein a mean aggregation number of the first particle and the second particle in the suspension is at most about 1,000, 100, or 10.

Embodiment 145. The composition of any one of Embodiments 135-144, wherein the suspension comprises at least about 1, 2, 3, 4, 5, 6, 7, 8, 9, or 10 parts of the first particle to about 1 part of the second particle.

Embodiment 146. The composition of any one of Embodiments 135-145, wherein the suspension comprises at most about 1, 2, 3, 4, 5, 6, 7, 8, 9, or 10 parts of the first particle to about 1 part of the second particle.

Embodiment 147. The composition of any one of Embodiments 135-146, wherein the suspension comprises about 15 parts of the first particle to about 6 parts of the second particle.

Embodiment 148. The composition of Embodiment 147, wherein the parts are parts by weight, parts by volume, or parts by surface area.

Embodiment 149. The composition of any one of Embodiments 135-148, wherein the suspension comprises at least about 3, 4, 5, 6, 7, 8, 9, or 10 distinct particles.

Embodiment 150. The composition of any one of Embodiments 135-149, wherein the first particle, the second particle, or both are a nanoparticle.

Embodiment 151. The composition of any one of Embodiments 135-150, wherein the first particle, the second particle, or both are a microparticle.

Embodiment 152. The composition of any one of Embodiments 135-151, wherein the first particle, the second particle, or both are porous.

Embodiment 153. The composition of any one of Embodiments 135-152, wherein a first size of the first particle is at least about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of the second particle.

Embodiment 154. The composition of 153, wherein a first size of the first particle is at most about 1.1, 1.2, 1.3, 1.4, 1.5, 1.6, 1.7, 1.8, 1.9, 2, 3, 4, 5, 10, 20, 30, 40, 50, 60, 70, 80, 90, 100, 200, 300, 400, 500, 600, 700, 800, 900, or 1,000 times a second size of the second particle.

Embodiment 155. The composition of any one of Embodiments 153-154, wherein the first size is a first diameter, and the second size is a second diameter.

Embodiment 156. The composition of any one of Embodiments 153-155, wherein the first size is a first average size, and the second size is a second average size.

Embodiment 157. The composition of Embodiment 156, wherein the first average size and the second average size are mean sizes or median sizes.

Embodiment 158. The composition of any one of Embodiments 135-152, wherein a ratio of a mean diameter for the first particle to a mean diameter of the second particle is about 3:2 to about 2:3.

Embodiment 159. The composition of any one of Embodiments 135-158, wherein the plurality of biomolecules comprises at most about 1,000, 100, 10, 1, 0.1, 0.01, or 0.001 nanograms of biomolecules.

Embodiment 160. The composition of any one of Embodiments 135-159, wherein the plurality of biomolecules comprises at most about 1,000, 100, 10, 1, 0.1, 0.01, or 0.001 nanograms of biomolecules per mL.

Embodiment 161. The composition of any one of Embodiments 135-160, wherein the plurality of biomolecules comprises biomolecules from at most about 1,000, 100, 10, or 1 cell.

Embodiment 162. A method comprising: assaying the plurality of biomolecules in the composition of any one of Embodiments 135-161 to identify one or more biomolecules in the plurality of biomolecules.

Embodiment 163. The method of Embodiment 162, wherein the assaying comprises performing mass spectrometry.

Embodiment 164. The method of Embodiment 163, wherein the mass spectrometry comprises LC-MS/MS.

Embodiment 165. The method of Embodiment 162, wherein the assaying comprises performing protein sequencing.

Embodiment 166. The method of any one of Embodiments 162-165, wherein the assaying comprises contacting the plurality of biomolecules with a pair of antibodies capable of binding to at least one biomolecule in the plurality of biomolecules, wherein the pair of antibodies comprises complementary single-stranded nucleic acid sequences attached thereto, such that when the pair of antibodies bind to the at least one biomolecule, the complementary nucleic acids hybridize to form a double stranded nucleic acid, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

Embodiment 167. The method of any one of Embodiments 162-166, wherein the assaying comprises contacting the plurality of biomolecules with one or more aptamers capable of binding to at least one biomolecule in the plurality of biomolecules, wherein the one or more aptamers are coupled to a surface via a cleavable linker.

Embodiment 168. The method of Embodiment 167, wherein the surface is a particle surface.

Embodiment 169. The method of Embodiment 167 or 168, wherein the cleavable linker is photocleavable.

Embodiment 170. The method of any one of Embodiments 162-169, further comprising contacting the plurality of biomolecules with a macromolecular competitor configured to, in a fluid composition, reduce dissociation of a complex comprising the one or more aptamers and the biomarker.

Embodiment 171. The method of Embodiment 170, wherein the macromolecular competitor is further configured to bind to a biomolecule that is different from the biomarker.

Embodiment 172. The method of Embodiment 171, wherein the macromolecular competitor is a polyanionic macromolecule.

Embodiment 173. The method of any one of Embodiments 167-172, wherein the assaying comprises performing nucleic acid sequencing.

Embodiment 174. The method of any one of Embodiments 162-173, wherein a sample comprises the plurality of biomolecules.

Embodiment 175. The method of Embodiment 174, wherein the sample is a complex biological sample.

Embodiment 176. The method of Embodiment 175, wherein the complex biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 177. The method of Embodiment 176, wherein the biological sample comprises plasma.

Embodiment 178. The method of any one of Embodiments 162-177, wherein the plurality of biomolecules comprises one or more polyamino acids.

Embodiment 179. The method of any one of Embodiments 162-178, wherein the assaying is performed at a rate of 1, 2, 3, 4, 5, 6, 7, 8, 9, 10, or 100 samples assayed per hour.

Embodiment 180. The method of any one of Embodiments 162-179, wherein the assaying is performed at a rate of at least 1, 10, 100, 1,000, 10,000, 100,000, 1,000,000, or 10,000,000 μg of biomolecules assayed per hour.

Embodiment 181. The method of any one of Embodiments 162-180, wherein the assaying is performed at a rate of at least 1, 10, 100, 1,000, 10,000, 100,000, or 1,000,000 biomolecules identified per hour.

Embodiment 182. The method of any one of Embodiments 162-181, wherein the assaying is performed at a rate of at least 1, 10, 100, 1,000, 10,000, 100,000, or 1,000,000 protein groups identified per hour.

Embodiment 183. The method of any one of Embodiments 162-182, wherein the assaying identifies at least about 100, 1,000, 10,000, or 100,000 biomolecules when the suspension comprises a HeLa cell extract.

Embodiment 184. The method of any one of Embodiments 162-183, wherein the plurality of biomolecules comprises at least about 6, 7, 8, 9, 10, 11, 12, 13, 14, 15, or 16 dynamic range when the assaying is performed on the plurality of biomolecules in a biological sample in the absence of the first particle and the second particle.

Embodiment 185. The method of any one of Embodiments 162-184, wherein a first likelihood that the assaying identifies a low abundance biomolecule in the plurality of biomolecules is larger than a second likelihood that the assaying identifies the low abundance biomolecule in a biological sample in the absence of the first particle and the second particle, wherein the low abundance biomolecule comprises less than about 1, 10⁻¹, 10⁻², 10⁻³, 10⁻⁴, 10⁻⁵, 10⁻⁶, 10⁻⁷, 10⁻⁸, 10⁻⁹, or 10⁻¹⁰ percent by mass of the first set of biomolecules or the second set of biomolecules.

Embodiment 186. The method of any one of Embodiments 162-185, wherein the first likelihood is larger than the second likelihood by a factor of at least 2, 5, 10, 102, 102, 103, 104, 105, 106, 107, 108, 109, or 10101.

Embodiment 187. The method of any one of Embodiments 162-186, wherein the assaying identifies one or more biomolecules in the plurality of biomolecules with a coefficient of variance of at most about 30, 25, 20, 15, 10, 5, 4, 3, 2, or 1 percent.

Embodiment 188. The method of any one of Embodiments 162-187, wherein the assaying identifies one or more biomolecules in the plurality of biomolecules with a coefficient of variance of at least about 30, 25, 20, 15, 10, 5, 4, 3, 2, or 1 percent.

Embodiment 189. A system for analyzing a plurality of biological samples, comprising: (a) a plurality of partitions comprising a first partition and a second partition; (b) a plurality of reagent storages comprising a first reagent comprising a first pH and a second reagent comprising a second pH, wherein the first pH and the second pH are different or the same; (c) a plurality of substrates comprising a first substrate comprising a first surface chemistry and a second substrate comprising a second surface chemistry; (d) one or more transfer devices operably connected to the plurality of partitions, the plurality of reagent storages, and the plurality of substrates; and (e) a computer comprising at least one processor and instructions executable by the at least one processor to perform steps comprising: i. generating, using the one or more transfer devices, a first fluid composition in the first partition comprising the first substrate, the first reagent, and a first plurality of biomolecules, wherein the first plurality of biomolecules is adsorbed on the first substrate; ii. generating, using the one or more transfer devices, a second fluid composition in the second partition comprising the second substrate, the second reagent, and a second plurality of biomolecules, wherein the second plurality of biomolecules is adsorbed on the second substrate; and iii. preparing, using the one or more transfer devices, the first plurality of biomolecules and the second plurality of biomolecules for mass spectrometry.

Embodiment 190. A system for analyzing a plurality of biological samples, comprising: (a) a partition; (b) a reagent storage comprising a reagent; (c) a plurality of substrates comprising a first substrate comprising a first surface chemistry and a second substrate comprising a second surface chemistry, wherein the first surface chemistry and the second surface chemistry are different; (d) one or more transfer devices operably connected to the partition, the reagent storage, and the plurality of substrates; and (e) a computer comprising at least one processor and instructions executable by the at least one processor to perform steps comprising: i. generating, using the one or more transfer devices, a fluid composition in the partition comprising the plurality of substrates, the reagent, and a plurality of biomolecules, wherein the plurality of biomolecules is adsorbed on the substrate; and ii. preparing, using the one or more transfer devices, the plurality of biomolecules for mass spectrometry.

Embodiment 191. A method comprising: (a) forming a first suspension comprising a first plurality of particles with a first portion of a biological sample, wherein the first plurality of particles comprise a first particle and second particle, wherein the first particle has a first functionalized surface chemistry that is different from a second functionalized surface chemistry of the second particle, and wherein both the first particle and the second particle have a negative charge; (b) forming a second suspension comprising a second plurality of particles with a second portion of the biological sample, wherein the second plurality of particles comprises a third particle and fourth particle, wherein third particle has a third functionalized surface chemistry that is different from a fourth functionalized surface chemistry of the fourth particle, and wherein both the third particle and the fourth particle have a positive charge; (c) enriching for biomolecules that have adsorbed to the first plurality of particles and the second plurality of parties; and (d) performing a downstream assay on the enriched biomolecules.

Embodiment 192. The method of Embodiment 191, wherein the first suspension and the second suspension each have a pH between 9 and 10.

Embodiment 193. The method of Embodiment 192, wherein the first suspension and the second suspension each comprise Tris buffer.

Embodiment 194. The method of Embodiment 191, wherein the first suspension has a pH between 9 and 10.

Embodiment 195. The method of Embodiment 194, wherein the first suspension comprises Tris buffer.

Embodiment 196. The method of Embodiment 191, 194, or 195, wherein the second suspension has a pH between 6 and 8.

Embodiment 197. The method of any one of Embodiments 191-196, wherein the first particle is a magnetic particle and comprises an outer layer with a silanol surface functionalization, and the second particle is a magnetic particle and comprises an outer layer with a carboxylate surface functionalization.

Embodiment 198. The method of any one of Embodiments 191-197, wherein the third particle is a magnetic particle and comprises an outer layer with an amine surface functionalization, and the fourth particle is a magnetic particle and comprises an outer layer with an amine surface functionalization.

Embodiment 199. The method of any one of Embodiments 191-198, wherein the outer layer of the third particle comprises a polymer.

Embodiment 200. The method of any one of Embodiments 191-199, wherein the first plurality of particles has a ratio of about 10 to about 15 parts by weight of the first particle to about 1 part by weight of the second particle.

Embodiment 201. The method of any one of Embodiments 191-200, wherein the second plurality of particles has a ratio about 10 to about 15 parts by weight of the third particle to about 1 part by weight of the fourth particle.

Embodiment 202. The method of any one of Embodiments 191-201, wherein a ratio of a mean diameter for the first particle to a mean diameter of the second particle is about 3:2 to about 2:3

Embodiment 203. The method of any one of Embodiments 191-202, wherein a ratio of a mean diameter for the third particle to a mean diameter of the fourth particle is about 3:2 to about 2:3.

Embodiment 204. The method of any one of Embodiments 191-203, wherein each of the first particle, the second particle, the third particle, and the fourth particle are superparamagnetic iron oxide nanoparticles.

Embodiment 205. A kit comprising: (a) a first composition comprising: a first particle and second particle, wherein first particle has a first functionalized surface chemistry that is different from a second functionalized surface chemistry of the second particle, and wherein both the first particle and the second particle have a negative charge, wherein the first composition is a solid; and (b) a second composition comprising: a third particle and fourth particle, wherein third particle has a third functionalized surface chemistry that is different from a fourth functionalized surface chemistry of the fourth particle, and wherein both the third particle and the fourth particle have a positive charge, wherein the second composition is a solid.

Embodiment 206. The kit of Embodiment 205, wherein the kit further comprises a buffer having a pH between 9 and 10.

Embodiment 207. The kit of Embodiment 205, wherein the kit further comprises a Tris buffer having a pH between 9 and 10.

Embodiment 208. The kit of Embodiment 205, wherein both the first composition and the second composition are lyophilized.

Embodiment 209. The kit of any one of Embodiments 205-208, wherein the first particle is a magnetic particle and comprises an outer layer with a silanol surface functionalization, and the second particle is a magnetic particle and comprises an outer layer with a carboxylate surface functionalization.

Embodiment 210. The kit of any one of Embodiments 205-209, wherein the third particle is a magnetic particle and comprises an outer layer with an amine surface functionalization, and the fourth particle is a magnetic particle and comprises an outer layer with an amine surface functionalization.

Embodiment 211. The kit of any one of Embodiments 205-210, wherein the outer layer of the third particle comprises a polymer.

Embodiment 212. The kit of any one of Embodiments 205-211, wherein the first composition has a ratio of about 10 to about 15 parts by weight of the first particle to about 1 part by weight of the second particle.

Embodiment 213. The kit of any one of Embodiments 205-212, wherein the second composition has a ratio of about 10 to about 15 parts by weight of the third particle to about 1 part by weight of the fourth particle.

Embodiment 214. The kit of any one of Embodiments 205-213, wherein a ratio of a mean diameter for the first particle to a mean diameter of the second particle is about 3:2 to about 2:3.

Embodiment 215. The kit of any one of Embodiments 205-214, wherein a ratio of a mean diameter for the third particle to a mean diameter of the fourth particle is about 3:2 to about 2:3.

Embodiment 216. The kit of any one of Embodiments 205-215, wherein each of the first particle, the second particle, the third particle, and the fourth particle are superparamagnetic iron oxide nanoparticles.

Embodiment 217. A suspension comprising: (a) first particles comprising an outer layer with a negatively charged surface functionalization; (b) a biological sample; and (c) a buffer configured to maintain a pH of the suspension between 9 and 10, wherein the biomolecules are adsorbed to the outer layer of the first particles.

Embodiment 218. A suspension comprising: (a) first particles comprising a first outer layer with a negatively charged surface functionalization; (b) second particles comprising a second outer layer with a negatively charged surface functionalization, wherein the second outer layer is different from the first outer layer; (c) a biological sample; and (d) a buffer configured to maintain a pH of the suspension between 9 and 10, wherein the biomolecules are adsorbed to the first outer layer of the first particles and the second outer layer of the particles.

Embodiment 219. The suspension of Embodiment 218, wherein the first outer layer comprises silanol, and the second outer layer comprises a carboxylate.

Embodiment 220. A suspension comprising: (a) first particles comprising a first outer layer with a positively charged surface functionalization; (b) second particles comprising a second outer layer with a positively charged surface functionalization, wherein the second outer layer is different from the first outer layer; (c) a biological sample; and (d) a buffer configured to maintain a pH of the suspension between 9 and 10, wherein the biomolecules are adsorbed to the first outer layer of the first particles and the second outer layer of the particles.

Embodiment 221. The suspension of Embodiment 220, wherein the first and second outer layer comprise an amine group.

Embodiment 222. The suspension of any one of Embodiments 217-221, wherein the biomolecules comprise at least 100 different peptides or proteins.

Embodiment 223. The suspension of any one of Embodiments 217-222, wherein the first particles and optionally the second particles are magnetic.

Embodiment 224. The suspension of any one of Embodiments 217-223, wherein the buffer is Tris buffer.

Embodiment 225. A method comprising: (a) incubating a composition in contact with one or more surfaces, wherein the composition comprises a biological sample and a buffer configured to maintain a pH of the composition between 8 and 11, wherein biomolecules from the biological sample are adsorbed onto the one or more surfaces; (b) enriching for the biomolecules that have adsorbed to the surfaces; (c) assaying the enriched biomolecules to identify one or more of the enriched biomolecules from the biological sample.

Embodiment 226. The method of Embodiment 225, wherein the buffer is configured to maintain a pH of the composition between 9 and 10.

Embodiment 227. The method of Embodiment 225, wherein the buffer is configured to maintain a pH of the composition of about 9.5.

Embodiment 228. The method of any one of Embodiments 225-227, wherein the buffer is Tris buffer.

Embodiment 229. The method of any one of Embodiments 225-207, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 230. The method of any one of Embodiments 225-229, wherein the biological sample is plasma or serum.

Embodiment 231. The method of any one of Embodiments 225-225, wherein the biological sample is cell culture media.

Embodiment 232. The method of any one of Embodiments 225-231, wherein the composition comprises no more than 60% by volume of the biological sample.

Embodiment 233. The method of any one of Embodiments 225-232, wherein the composition comprises no more than 25% by volume of the biological sample.

Embodiment 234. The method of any one of Embodiments 225-233, wherein the composition comprises no more than 15% by volume of the biological sample.

Embodiment 235. The method of any one of Embodiments 225-234, wherein the composition comprises at least 1% by volume of the biological sample.

Embodiment 236. The method of any one of Embodiments 225-235, wherein the composition comprises at least 4% by volume of the biological sample.

Embodiment 237. The method of any one of Embodiments 225-236, wherein the composition comprises at least 10% by volume of the biological sample.

Embodiment 238. The method of any one of Embodiments 225-237, wherein the composition comprises at least 20% by volume of the biological sample.

Embodiment 239. The method of any one of Embodiments 225-238, wherein the assaying comprises SDS-PAGE, gel-based separation techniques, an immunoassay, ELISA, high performance liquid chromatography, mass spectrometry, Edman Degradation, or immunoaffinity techniques.

Embodiment 240. The method of any one of Embodiments 225-238, wherein the assaying comprises mass spectrometry.

Embodiment 241. The method of any one of Embodiments 225-238, wherein the assaying comprises LC-MS/MS.

Embodiment 242. The method of any one of Embodiments 225-241, wherein the assaying comprises quantifying the biomolecules.

Embodiment 243. The method of any one of Embodiments 225-242, wherein the assaying comprises identifying one or more proteins in the biological sample.

Embodiment 244. The method of Embodiment 243, wherein at least 100 different proteins are identified in the biological sample.

Embodiment 245. The method of Embodiment 243, wherein at least 250 different proteins are identified in the biological sample.

Embodiment 246. The method of Embodiment 243, wherein at least 500 different proteins are identified in the biological sample.

Embodiment 247. The method of any one of Embodiments 225-246, wherein the one or more surfaces are comprised on particles dispersed in the composition during the incubation.

Embodiment 248. The method of Embodiment 247, where the particles are nanoparticles.

Embodiment 249. The method of Embodiment 248, wherein the nanoparticles have an average diameter of less than 500 nanometers.

Embodiment 250. The method of Embodiment 247, where the particles are microparticles.

Embodiment 251. The method of any one of Embodiments 247-250, wherein the particles are magnetic.

Embodiment 252. The method of any one of Embodiments 247-251, wherein the particles comprise an outer layer of poly(dimethyl aminopropyl methacrylamide) (PDMAPMA).

Embodiment 253. The method of any one of Embodiments 247-251, wherein the particles comprise an outer layer of silica.

Embodiment 254. The method of any one of Embodiments 247-251, wherein the particles comprise a carboxylate-functionalized outer layer.

Embodiment 255. The method of any one of Embodiments 247-251, wherein the particles comprise an amine-functionalized outer layer.

Embodiment 256. The method of any one of Embodiments 247-251, wherein the particles comprise a polymeric outer layer.

Embodiment 257. The method of any one of Embodiments 247-251, wherein the particles comprise a polyacrylamide outer layer.

Embodiment 258. The method of any one of Embodiments 247-251, wherein the particles comprise an acrylate polymer.

Embodiment 259. The method of any one of Embodiments 225-258, the method further comprising: (a) incubating a second composition in contact with one or more second surfaces, wherein the second composition comprises the biological sample, wherein biomolecules from the biological sample are adsorbed onto the one or more second surfaces; (b) enriching for the biomolecules that have adsorbed to the one or more second surfaces; (c) assaying the enriched biomolecules to identify one or more of the enriched biomolecules from the one or more second surfaces in the biological sample.

Embodiment 260. The method of Embodiment 259, wherein the second composition has a pH between 5 and 9.

Embodiment 261. The method of Embodiment 259, wherein the second composition has a pH between 6 and 8.

Embodiment 262. The method of Embodiment 259, wherein the second composition has a pH between 8 and 11.

Embodiment 263. The method of Embodiment 259, wherein the second composition has a pH between 9 and 10.

Embodiment 264. The method of Embodiment 259, wherein the second composition does not comprise a buffer.

Embodiment 265. The method of Embodiment 259, wherein the second composition comprises a buffer that is different from the buffer configured to maintain a pH of the composition between 8 and 11.

Embodiment 266. The method of Embodiment 259, wherein the second composition comprises the same buffer as the composition.

Embodiment 267. The method of Embodiment 259, wherein the composition and the second composition both comprise Tris buffer having a pH between 9 and 10.

Embodiment 268. The method of any one of Embodiments 259-267, wherein the incubating of the composition and the incubating of the second composition occur at the same time.

Embodiment 269. The method of any one of Embodiments 259-268, wherein the assay identifies the enriched biomolecules with a coefficient of variance of at most 30%.

Embodiment 270. The method of any one of Embodiments 259 to 268, wherein the assay identifies the enriched biomolecules with a coefficient of variance of at most 25%.

Embodiment 271. The method of any one of Embodiments 259-249, wherein the method further comprises digesting proteins adsorbed to the first surfaces before assaying the biomolecules.

Embodiment 272. The method of any one of Embodiments 259-271, wherein the method further comprises treating proteins within the biomolecule corona with a reducing agent before assaying the biomolecules.

Embodiment 273. The method of any one of Embodiments 259-272, wherein the method further comprises treating proteins within the biomolecule corona with an alkylating agent before assaying the biomolecules.

Embodiment 274. A suspension comprising: (a) a first particle having a first functionalized surface chemistry; (b) a second particle having a second functionalized surface chemistry, wherein the first functionalized surface chemistry is different from the second functionalized surface chemistry of the second particle, and wherein both the first particle and the second particle have a negative charge; (c) a biological sample; and (d) a buffer configured to maintain a pH of about 8 to about 11.

Embodiment 275. The suspension of Embodiment 274, wherein the buffer is configured to maintain a pH between 9 and 10.

Embodiment 276. The suspension of Embodiment 274, wherein the buffer is Tris buffer having a pH between 9 and 10.

Embodiment 277. The suspension of any one of Embodiments 274-276, wherein the first particle is a magnetic particle and comprises an outer layer with a silanol surface functionalization, and the second particle is a magnetic particle and comprises an outer layer with a carboxylate surface functionalization.

Embodiment 278. The suspension of any one of Embodiments 274-277, wherein the first composition has a ratio of about 10 to about 15 parts by weight of the first particle to about 1 part by weight of the second particle.

Embodiment 279. The suspension of any one of Embodiments 274-278, wherein a ratio of a mean diameter for the first particle to a mean diameter of the second particle is about 3:2 to about 2:3.

Embodiment 280. The suspension of any one of Embodiments 274-279, wherein the first particle and the second particle are superparamagnetic iron oxide nanoparticles.

Embodiment 281. A suspension comprising: (a) a first particle having a first functionalized surface chemistry; (b) a second particle having a second functionalized surface chemistry, wherein the first functionalized surface chemistry is different from the second functionalized surface chemistry of the second particle, and wherein both the first particle and the second particle have a positive charge; and (c) a biological sample.

Embodiment 282. The suspension of Embodiment 281, wherein the first particle is a magnetic particle and comprises an outer layer with an amine surface functionalization, and the second particle is a magnetic particle and comprises an outer layer with an amine surface functionalization.

Embodiment 283. The suspension of any one of Embodiments 281-282, wherein the outer layer of the first particle comprises a polymer.

Embodiment 284. The suspension of any one of Embodiments 281-283, wherein the suspension has a ratio of about 10 to about 15 parts by weight of the first particle to about 1 part by weight of the second particle.

Embodiment 285. The suspension of any one of Embodiments 281-284, wherein a ratio of a mean diameter for the first particle to a mean diameter of the second particle is about 3:2 to about 2:3.

Embodiment 286. The suspension of any one of Embodiments 281-285, wherein the first particle and the second particle are superparamagnetic iron oxide nanoparticles.

Embodiment 287. The suspension of any one of Embodiments 274-286, wherein the biological sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 288. The suspension of any one of Embodiments 274-286, wherein the biological sample comprises plasma or serum.

Embodiment 289. The suspension of any one of Embodiments 274-288, wherein the biological sample is a cell-free biological sample.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1.-28. (canceled)

29. A method comprising:
(a) selectively enriching a first plurality of biomolecule types in a first fluid composition, wherein the selectively enriching the first plurality of biomolecule types in the first fluid composition comprises contacting the first fluid composition with a first surface to adsorb the first plurality of biomolecule types on the first surface;
(b) selectively enriching a second plurality of biomolecule types in a second fluid composition, wherein the selectively enriching the second plurality of biomolecule types in the second fluid composition comprises contacting the second fluid composition with a second surface to adsorb the second plurality of biomolecule types on the second surface;
(c) washing the first plurality of biomolecule types with a first wash composition and washing the second plurality of biomolecule types with a second wash composition; and
(d) performing a downstream assay on biomolecule types of the first plurality of biomolecule types and the second plurality of biomolecule types, wherein the downstream assay comprises mass spectrometry, wherein the first fluid composition comprises a first pH, and the second fluid composition comprises a second pH, wherein the first pH and the second pH are different or the same.

30. The method of claim 29, wherein the first fluid composition and the second fluid composition comprise different solvents.

31. The method of claim 29, wherein the first fluid composition comprises a buffer selected from the group consisting of tris(hydroxymethyl) aminomethane, glycine, phosphate, and N-cyclohexyl-3-aminopropanesulfonic acid.

32. The method of claim 31, wherein the buffer comprises tris(hydroxymethyl) aminomethane.

33. The method of claim 29, wherein the second fluid composition comprises a buffer selected from the group consisting of tris(hydroxymethyl) aminomethane, glycine, phosphate, and N-cyclohexyl-3-aminopropanesulfonic acid.

34. The method of claim 33, wherein the buffer comprises tris(hydroxymethyl) aminomethane.

35. The method of claim 29, wherein the first fluid composition, the second fluid composition, or both comprise a pH of at least about 8.

36. The method of claim 29, wherein the first fluid composition, the second fluid composition, or both comprise a pH between 9 and 10.

37. The method of claim 29, further comprising diluting a sample to make the first fluid composition, the second fluid composition, or both.

38. The method of claim 37, wherein the diluting comprises adding a buffer.

39. The method of claim 38, wherein the buffer comprises a pH of at least about 8.

40. The method of claim 29, wherein the first plurality of biomolecule types, the second plurality of biomolecule types, or both comprise one or more polyamino acids.

41. The method of claim 29, wherein the first fluid composition and the second fluid composition are from different samples.

42. The method of claim 29, wherein the biological sample comprises plasma.

43. The method of claim 29, wherein the first surface, the second surface, or both comprise a particle.

44. The method of claim 43, wherein the particle is a microparticle or nanoparticle.

45. The method of claim 43, wherein the particle comprises a paramagnetic material.

46. The method of claim 29, wherein the first plurality of surface types, the second plurality of surface types, or both comprise the same sign of zeta potential.

47. The method of claim 29, wherein the first plurality of surface types, the second plurality of surface types, or both comprise an acidic functional group.

48. The method of claim 29, wherein the mass spectrometry comprises LC-MS/MS.
